(19) United States
(12) Patent Application Publication
(10) Pub. No.: US 2024/0383870 A1
(43) Pub. Date: Nov. 21, 2024

(54) HETEROCYCLIC ALDEHYDE TRAPPING COMPOUNDS AND USES THEREOF

(71) Applicant: Aldeyra Therapeutics, Inc., Lexington, MA (US)

(72) Inventors: Adam BROCKMAN, Arlington, MA (US); Stephen Gitu MACHATHA, Wilmington, MA (US); Charles MONTGOMERY, Bolton, MA (US)

(21) Appl. No.: 18/568,566

(22) PCT Filed: Jul. 1, 2022
(86) PCT No.: PCT/US22/35898

§ 371 Date: Dec. 8, 2023

Related U.S. Application Data
(60) Provisional application No. 63/202,979, filed on Jul. 2, 2021.

(51) Int. Cl. C07D 333/66; A61K 31/136; A61K 31/343; A61K 31/357; A61K 31/381; A61K 31/416; A61K 31/44; A61K 31/517; C07C 215/68; C07C 317/32; C07D 213/16; C07D 231/56; C07D 241/44; C07D 307/79; C07D 307/82; C07D 317/46; C07D 319/16; C07D 339/04

(52) U.S. Cl. CPC C07D 333/66; A61K 31/136; A61K 31/343; A61K 31/357; A61K 31/381; A61K 31/416; A61K 31/44; A61K 31/517; C07C 215/68; C07C 317/32; C07D 213/16; C07D 231/56; C07D 241/44; C07D 307/79; C07D 307/82; C07D 317/46; C07D 319/16; C07D 339/04

(57) ABSTRACT

The present invention provides for the treatment, prevention, and/or reduction of a risk of a disease, disorder, or condition in which aldehyde toxicity is implicated in the pathogenesis, including ocular disorders, skin disorders, conditions associated with injurious effects from blister agents, and autoimmune, inflammatory, neurological and cardiovascular diseases by the use of a disclosed compound or a pharmaceutically acceptable salt thereof.

### TECHNICAL FIELD OF THE INVENTION

The present invention relates to compounds useful for trapping disease-causing aldehydes. The present invention further relates to methods of use of such compounds for treating a disease, disorder, or condition such as those described herein, as well as pharmaceutical compositions of such compounds.

### CROSS-REFERENCE TO RELATED APPLICATIONS

This application claims the benefit of priority to U.S. Provisional Patent Application Ser. No. 63/202,979, filed Jul. 2, 2021, the entirety of which is hereby incorporated by reference.

### BACKGROUND OF THE INVENTION

Metabolic and inflammatory processes in cells generate toxic aldehydes, such as malondialdehyde (MDA) and 4-hydroxyl-2-nonenal (4HNE). These aldehydes are highly reactive to proteins, carbohydrates, lipids and DNA, leading to chemically modified biological molecules, activation of inflammatory mediators such as NF-kappaB, and damage in diverse organs. For example, retinaldehyde can react with phosphatidylethanolamine (PE) to form a highly toxic compound called A2E, which is a component of lipofuscin believed to be involved in the development and progression of Age Related Macular Degeneration (AMD). Many bodily defense mechanisms function to remove or lower the levels of toxic aldehydes. Novel small molecule therapeutics can be used to scavenge “escaped” retinaldehyde in the retina, thus reducing A2E formation and lessening the risk of AMD.

Aldehydes are implicated in diverse pathological conditions such as dry eye, cataracts, keratoconus, Fuch's endothelial dystrophy in the cornea, uveitis, allergic conjunctivitis, ocular cicatricial pemphigoid, conditions associated with photorefractive keratectomy (PRK) healing or other corneal healing, conditions associated with tear lipid degradation or lacrimal gland dysfunction, inflammatory ocular conditions such as ocular rosacea (with or without meibomian gland dysfunction), and non-ocular disorders or conditions such as skin cancer, psoriasis, contact dermatitis, atopic dermatitis, acne vulgaris, Sjogren-Larsson Syndrome, ischemic-reperfusion injury, inflammation, diabetes, neurodegeneration (e.g., Parkinson's disease), scleroderma, amyotrophic lateral sclerosis, autoimmune disorders (e.g., lupus), cardiovascular disorders (e.g., atherosclerosis), and conditions associated with the injurious effects of blister agents (Negre-Salvagre et al. (2008), Nakamura et al. (2007), Batista et al. (2012), Kenney et al. (2003), Int J Dermatol 43: 494 (2004), Invest Ophthalmol Vis Sci 48: 1552 (2007), Graefe's Clin Exp Ophthalmol 233: 694 (1994), Molecular Vision 18: 194 (2012)). Reducing or eliminating aldehydes should thus ameliorate the symptoms and slow the progression of these pathological conditions.

MDA, HNE and other toxic aldehydes are generated by a myriad of metabolic mechanisms involving: fatty alcohols, sphingolipids, glycolipids, phytol, fatty acids, arachadonic acid metabolism (Rizzo (2007)), polyamine metabolism (Wood et al. (2006)), lipid peroxidation, oxidative metabolism (Buddi et al. (2002), Zhou et al. (2005)), and glucose metabolism (Pozzi et al. (2009)). Aldehydes can cross link with primary amino groups and other chemical moieties on proteins, phospholipids, carbohydrates, and DNA, leading in many cases to toxic consequences, such as mutagenesis and carcinogenesis (Marnett (2002)). MDA is associated with diseased corneas, keratoconus, bullous and other keratopathy, and Fuch's endothelial dystrophy corneas (Buddi et al. (2002)). Also, skin disorders, e.g., Sjogren-Larsson Syndrome, are likely connected with the accumulation of fatty aldehydes such as octadecanal and hexadecanal (Rizzo et al. (2010)). Further, increased lipid peroxidation and resultant aldehyde generation are associated with the toxic effects of blister agents (Sciuto et al. (2004) and Pal et al. (2009)).

Accordingly, there remains a need for treating, preventing, and/or reducing a risk of a disease, disorder, or condition in which aldehyde toxicity is implicated in the pathogenesis.

### SUMMARY OF THE INVENTION

It has now been found that compounds of the present invention, and compositions thereof, are useful for treating, preventing, and/or reducing a risk of a disease, disorder, or condition in which aldehyde toxicity is implicated in the pathogenesis. In one aspect of the present invention, such compounds have general formula I:

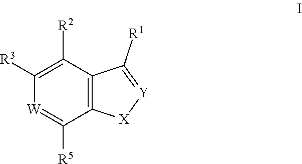

or a pharmaceutically acceptable salt thereof, wherein each of X, Y, W, R¹, R², R³, and R⁵ is as defined herein.

In another aspect, the present invention provides a compound of formula VI:

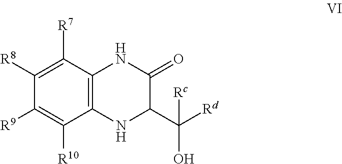

or a pharmaceutically acceptable salt thereof, wherein each of R^{c}, R^{d}, R⁷, R⁸, R⁹, and R¹⁰ is as defined herein.

Compounds of the present invention, and pharmaceutically acceptable compositions thereof, are useful for treating a variety of diseases, disorders or conditions, associated with toxic aldehydes. Such diseases, disorders, or conditions include those described herein.

Compounds provided by this invention are also useful for the study of certain aldehydes in biology and pathological phenomena.

### BRIEF DESCRIPTION OF THE FIGURES

FIG. 1 is a graph showing the results of an eotaxin cytokine detection assay in samples collected from mouse subjects after administration of compounds of the present disclosure.

FIG. 2 is a graph showing the results of a G-CSF cytokine detection assay in samples collected from mouse subjects after administration of compounds of the present disclosure.

FIG. 3 is a graph showing the results of a GM-CSF cytokine detection assay in samples collected from mouse subjects after administration of compounds of the present disclosure.

FIG. 4 is a graph showing the results of an IFNγ cytokine detection assay in samples collected from mouse subjects after administration of compounds of the present disclosure.

FIG. 5 is a graph showing the results of an IL-1α cytokine detection assay in samples collected from mouse subjects after administration of compounds of the present disclosure.

FIG. 6 is a graph showing the results of an IL-1β cytokine detection assay in samples collected from mouse subjects after administration of compounds of the present disclosure.

FIG. 7 is a graph showing the results of an IL-2 cytokine detection assay in samples collected from mouse subjects after administration of compounds of the present disclosure.

FIG. 8 is a graph showing the results of an IL-3 cytokine detection assay in samples collected from mouse subjects after administration of compounds of the present disclosure.

FIG. 9 is a graph showing the results of an IL-4 cytokine detection assay in samples collected from mouse subjects after administration of compounds of the present disclosure.

FIG. 10 is a graph showing the results of an IL-5 cytokine detection assay in samples collected from mouse subjects after administration of compounds of the present disclosure.

FIG. 11 is a graph showing the results of an IL-6 cytokine detection assay in samples collected from mouse subjects after administration of compounds of the present disclosure.

FIG. 12 is a graph showing the results of an IL-7 cytokine detection assay in samples collected from mouse subjects after administration of compounds of the present disclosure.

FIG. 13 is a graph showing the results of an IL-9 cytokine detection assay in samples collected from mouse subjects after administration of compounds of the present disclosure.

FIG. 14 is a graph showing the results of an IL-10 cytokine detection assay in samples collected from mouse subjects after administration of compounds of the present disclosure.

FIG. 15 is a graph showing the results of an IL-12(p40) cytokine detection assay in samples collected from mouse subjects after administration of compounds of the present disclosure.

FIG. 16 is a graph showing the results of an IL-12(p70) cytokine detection assay in samples collected from mouse subjects after administration of compounds of the present disclosure.

FIG. 17 is a graph showing the results of an IL-13 cytokine detection assay in samples collected from mouse subjects after administration of compounds of the present disclosure.

FIG. 18 is a graph showing the results of an IL-15 cytokine detection assay in samples collected from mouse subjects after administration of compounds of the present disclosure.

FIG. 19 is a graph showing the results of an IL-17 cytokine detection assay in samples collected from mouse subjects after administration of compounds of the present disclosure.

FIG. 20 is a graph showing the results of a KC cytokine detection assay in samples collected from mouse subjects after administration of compounds of the present disclosure.

FIG. 21 is a graph showing the results of a leukemia inhibitory factor (LIF) cytokine detection assay in samples collected from mouse subjects after administration of compounds of the present disclosure. LIF is an interleukin 6 class cytokine that affects cell growth by inhibiting differentiation.

FIG. 22 is a graph showing the results of a LIX (CXCL5) cytokine detection assay in samples collected from mouse subjects after administration of compounds of the present disclosure.

FIG. 23 is a graph showing the results of an MCP-1(CCL2) cytokine detection assay in samples collected from mouse subjects after administration of compounds of the present disclosure. MCP-1 recruits monocytes, memory T cells, and dendritic cells to the sites of inflammation.

FIG. 24 is a graph showing the results of an M-CSF (CSF1) cytokine detection assay in samples collected from mouse subjects after administration of compounds of the present disclosure. M-CSF causes hematopoietic stem cells to differentiate into macrophages.

FIG. 25 is a graph showing the results of an MIP-1a(CCL3) cytokine detection assay in samples collected from mouse subjects after administration of compounds of the present disclosure.

FIG. 26 is a graph showing the results of a MIP-1b(CCL4) cytokine detection assay in samples collected from mouse subjects after administration of compounds of the present disclosure.

FIG. 27 is a graph showing the results of a MIP2(CXCL2) cytokine detection assay in samples collected from mouse subjects after administration of compounds of the present disclosure.

FIG. 28 is a graph showing the results of a RANTES(CCL) cytokine detection assay in samples collected from mouse subjects after administration of compounds of the present disclosure.

FIG. 29 is a graph showing the results of a TNFα cytokine detection assay in samples collected from mouse subjects after administration of compounds of the present disclosure.

FIG. 30 is a graph showing the results of a VEGF cytokine detection assay in samples collected from mouse subjects after administration of compounds of the present disclosure.

FIG. 31 is a plot of HNE binding over time for various compounds of the present disclosure.

FIG. 32 is a plot of % completion of HNE binding over time for various compounds of the present disclosure.

FIG. 33 is a plot of % completion of HNE binding over time for various compounds of the present disclosure.

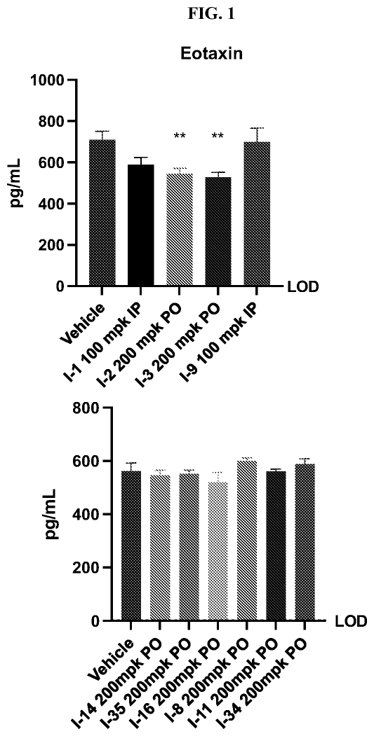

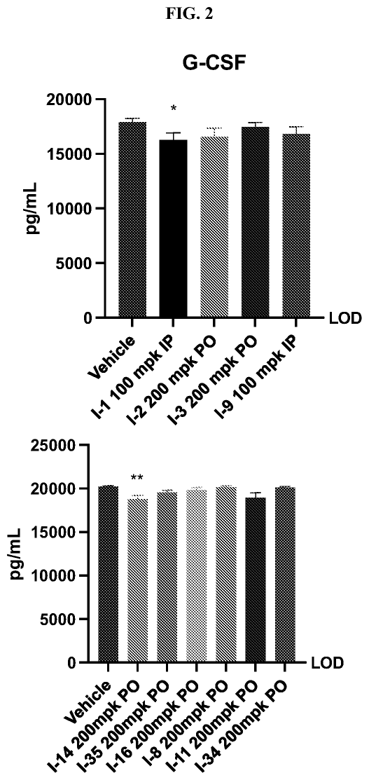

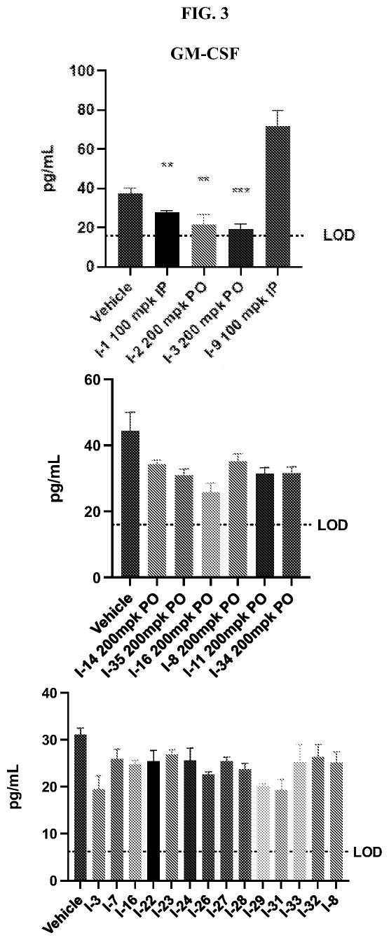

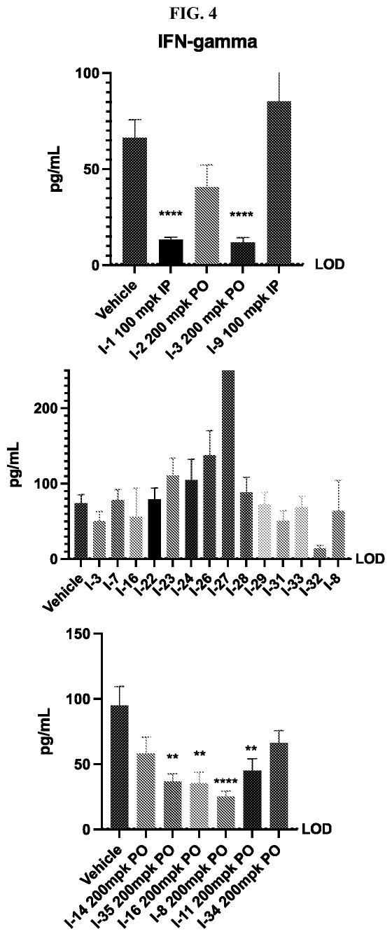

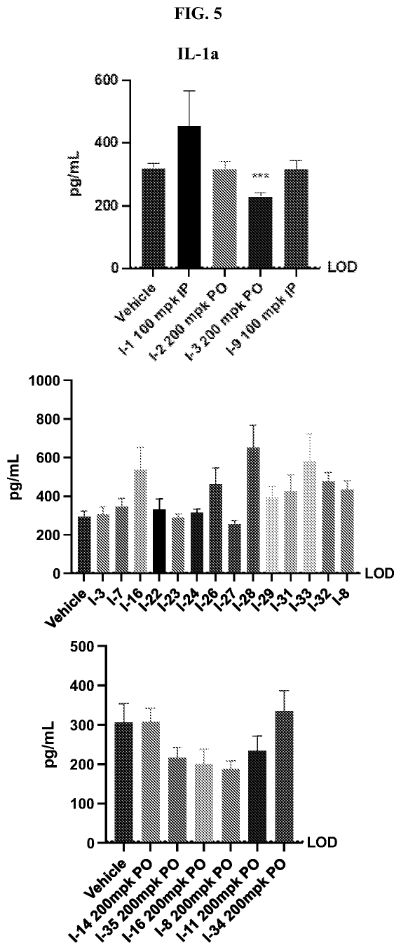

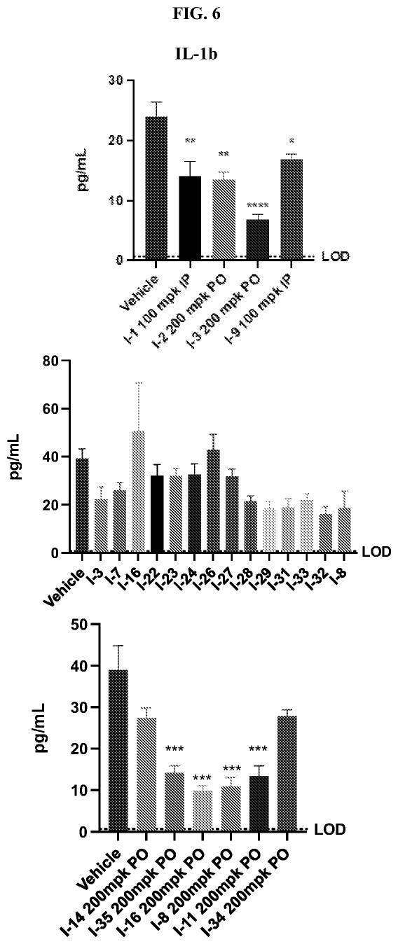

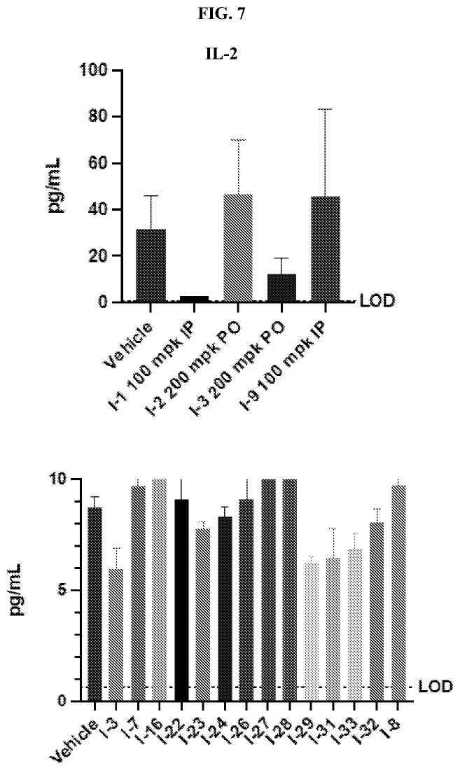

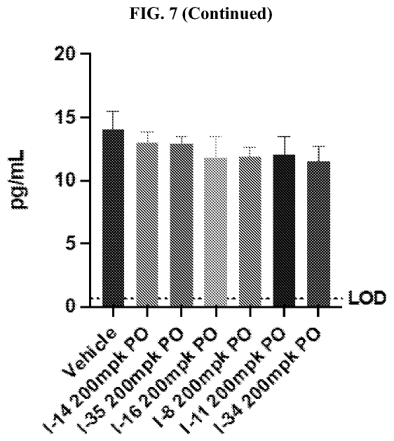

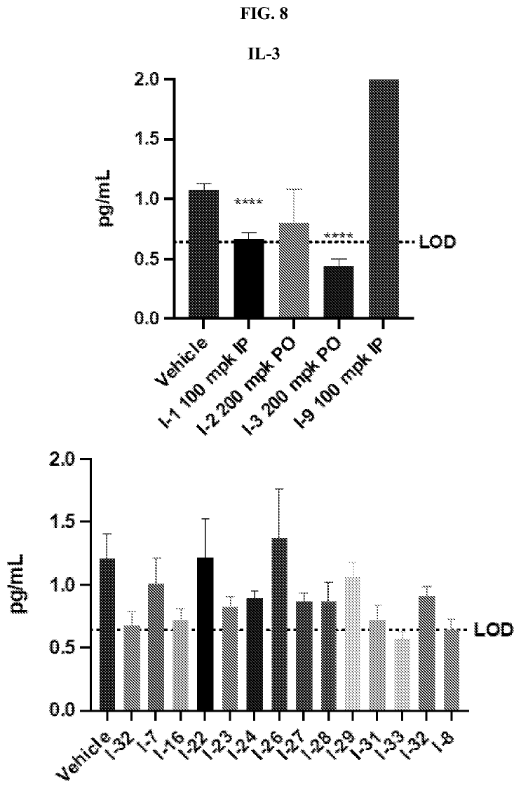

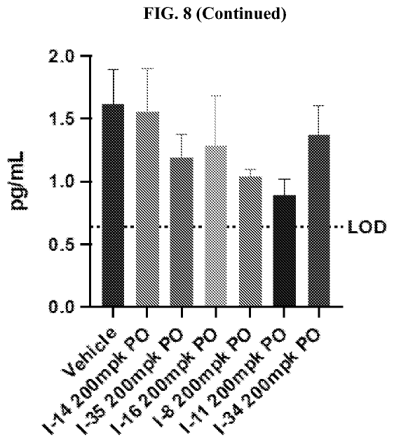

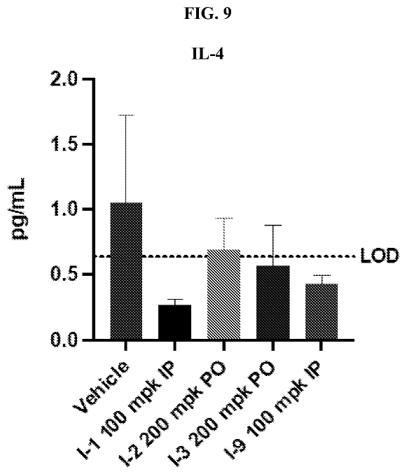

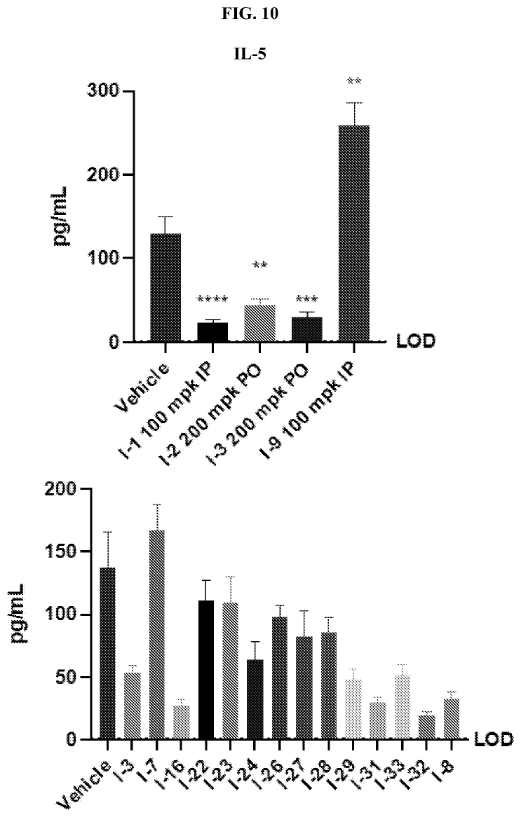

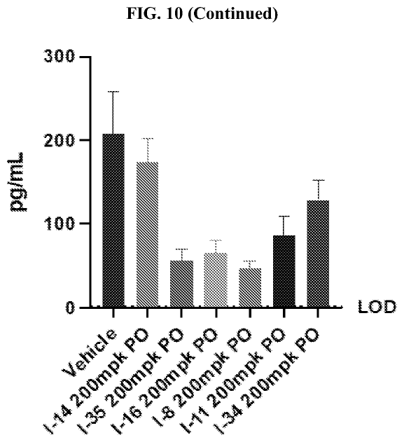

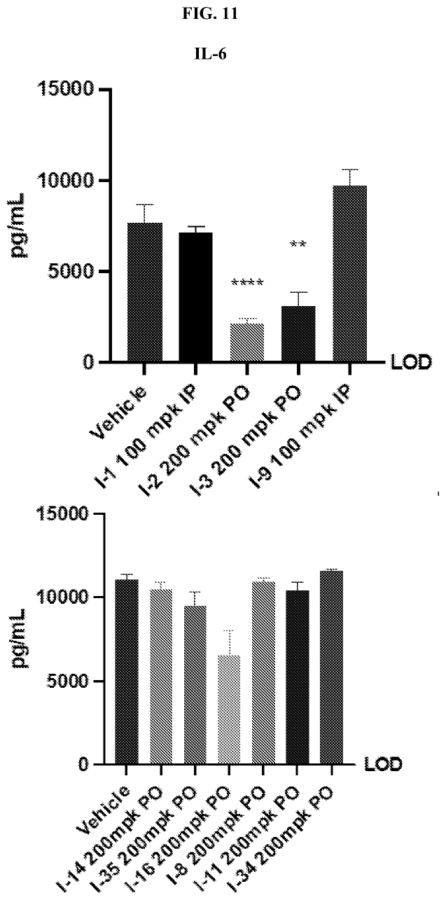

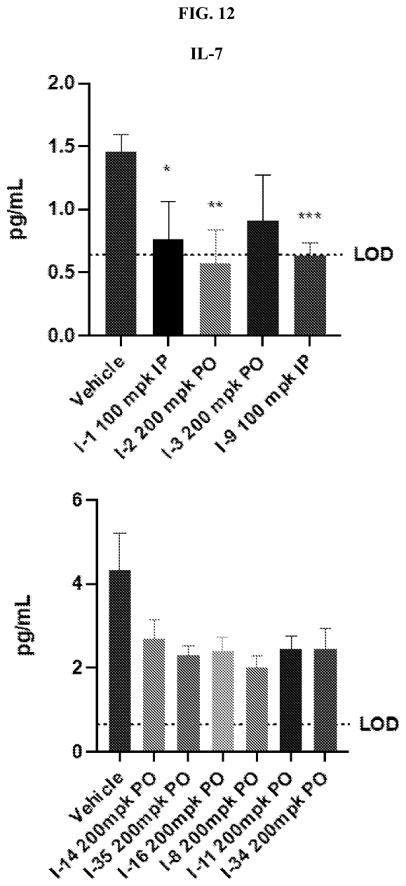

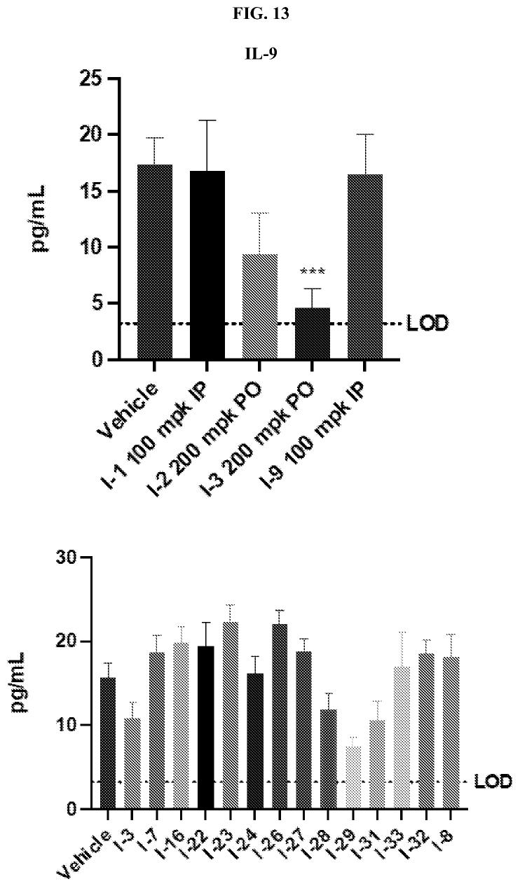

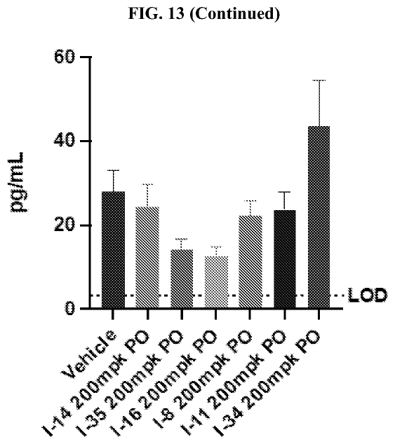

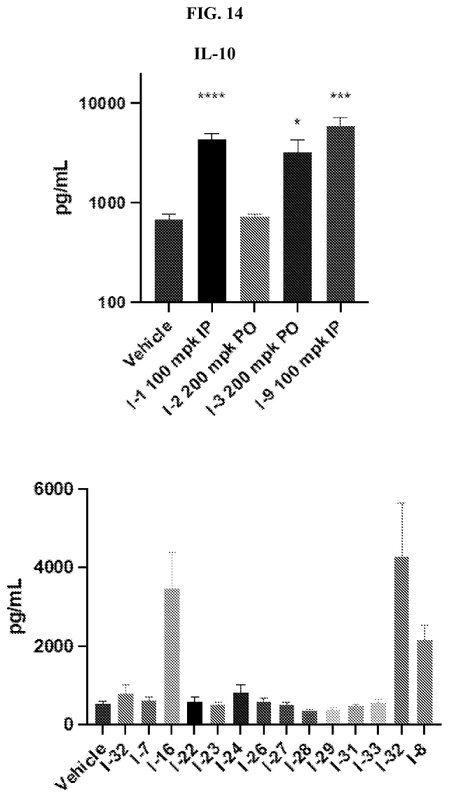

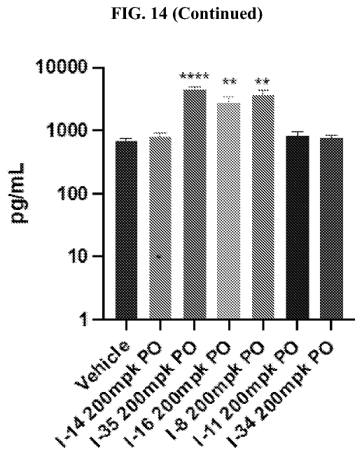

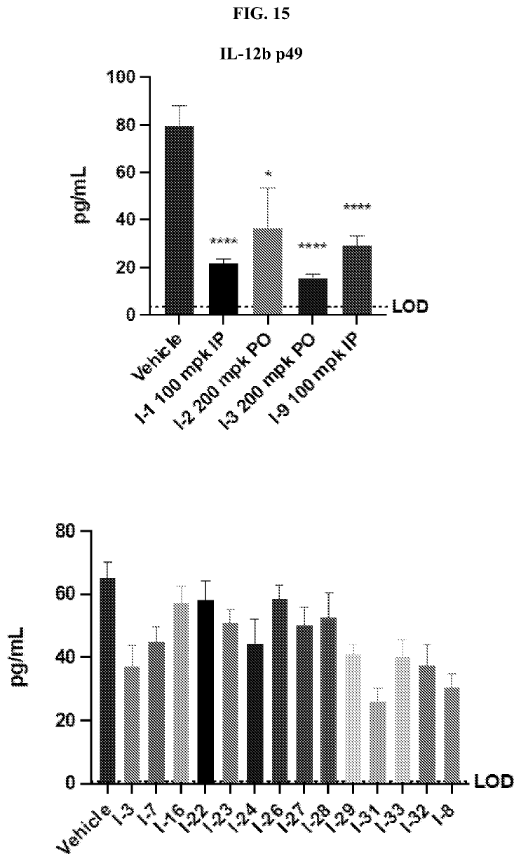

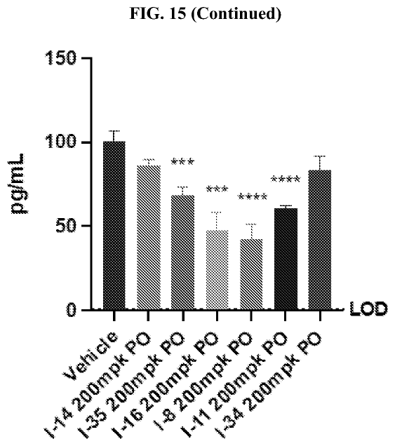

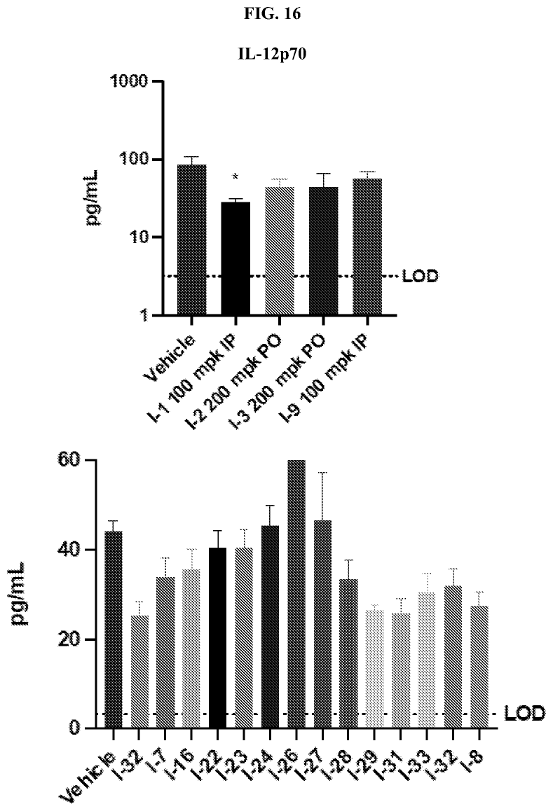

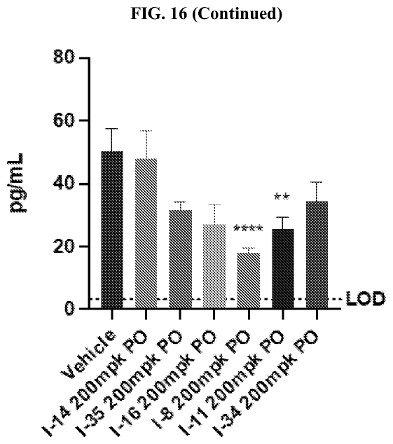

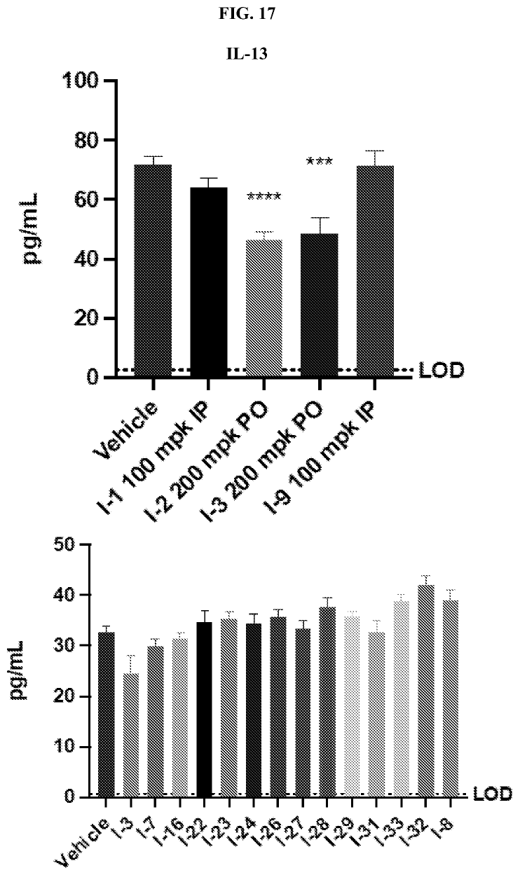

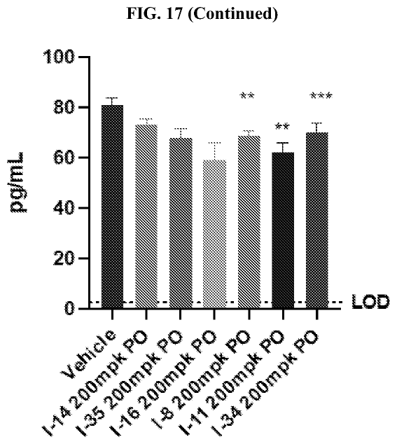

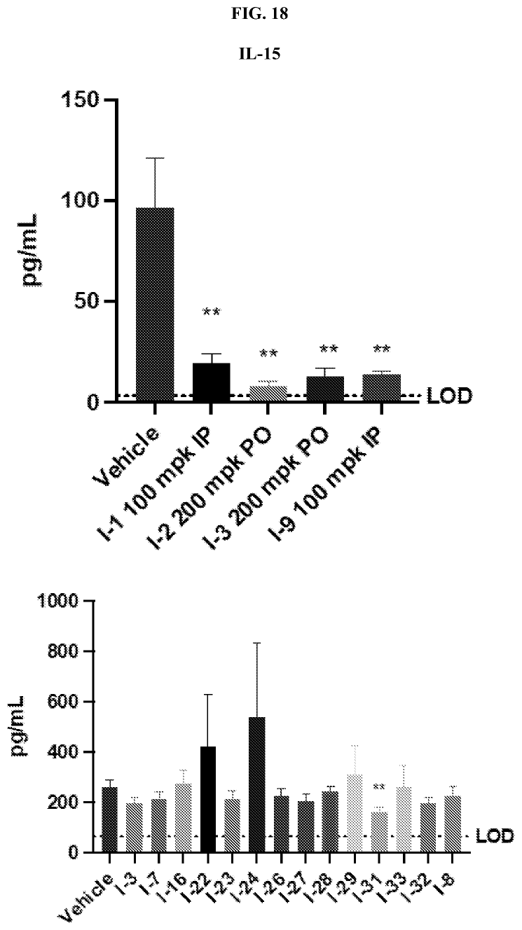

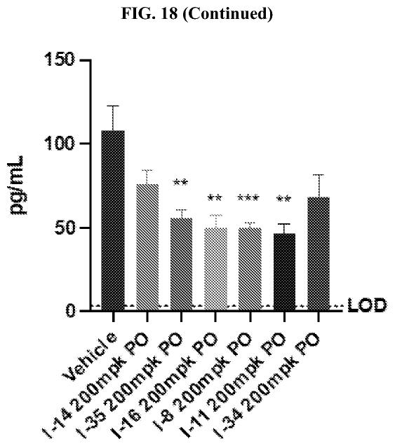

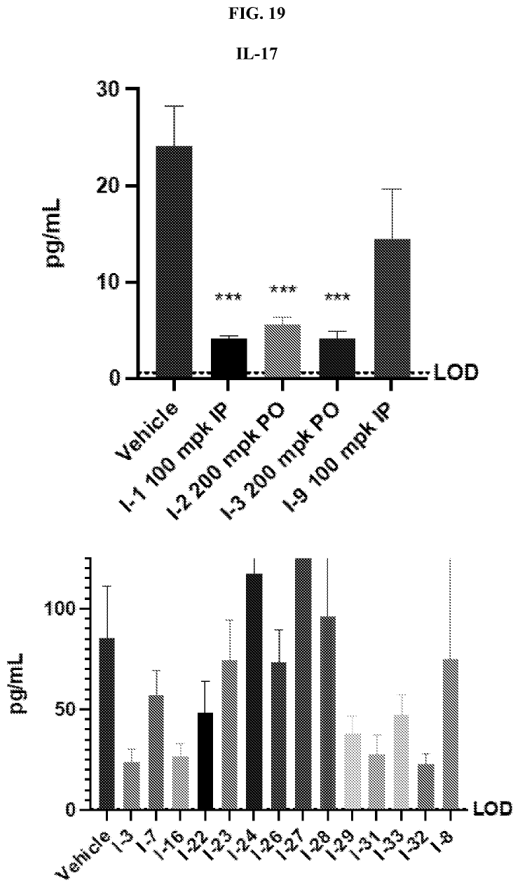

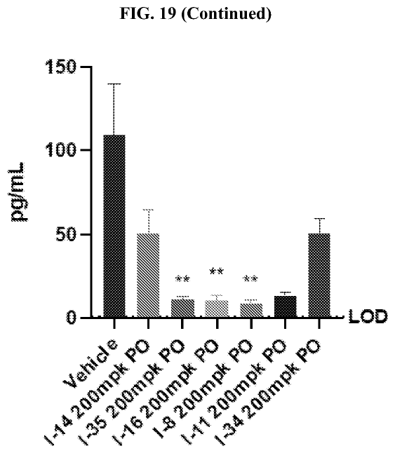

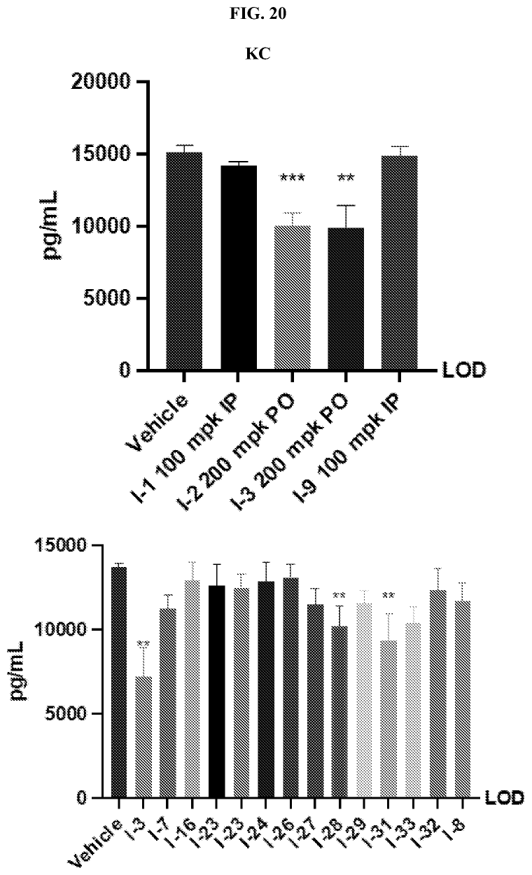

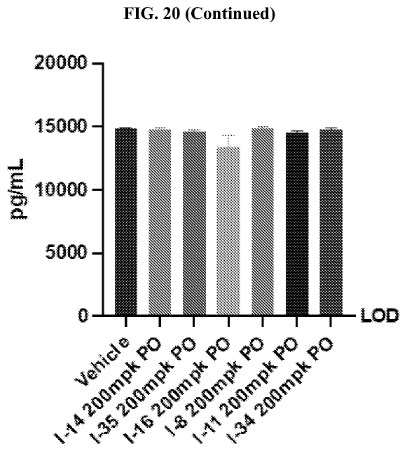

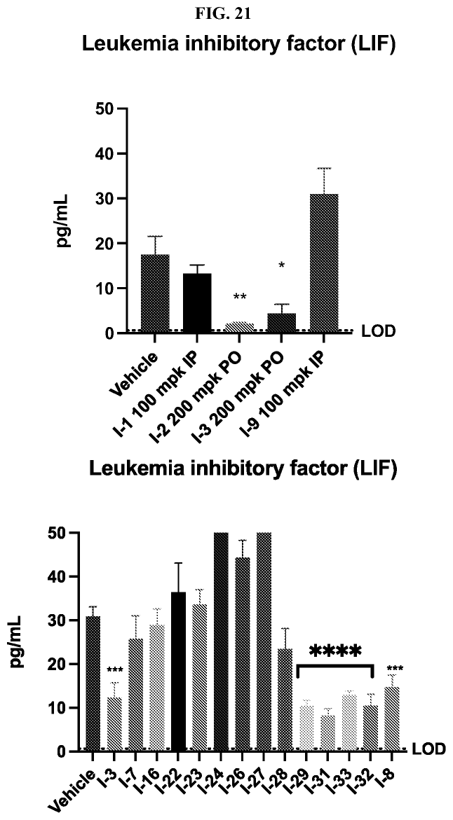

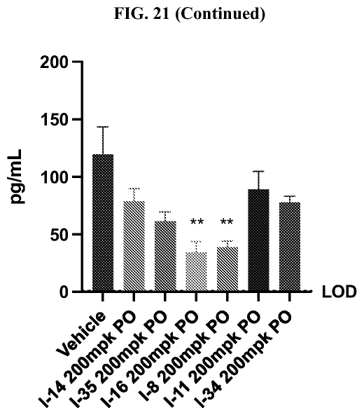

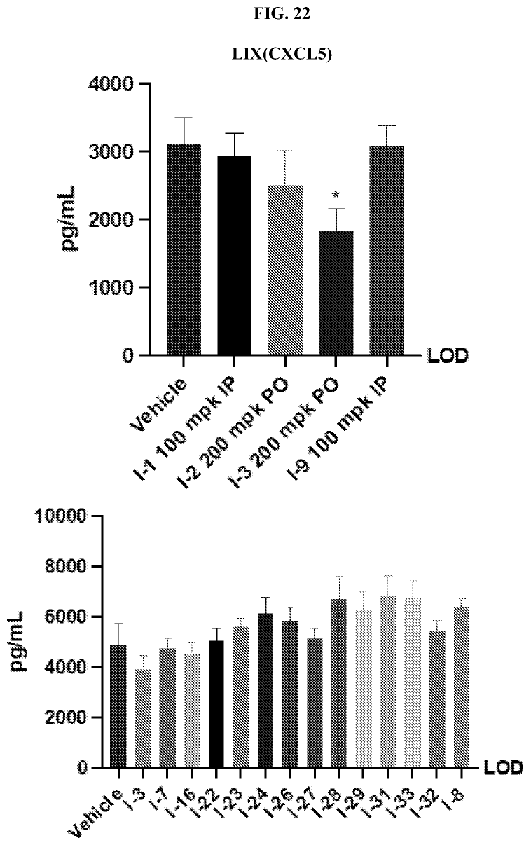

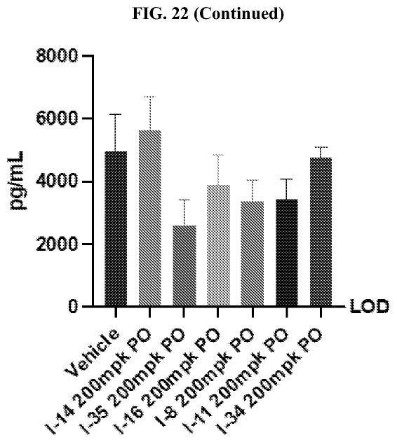

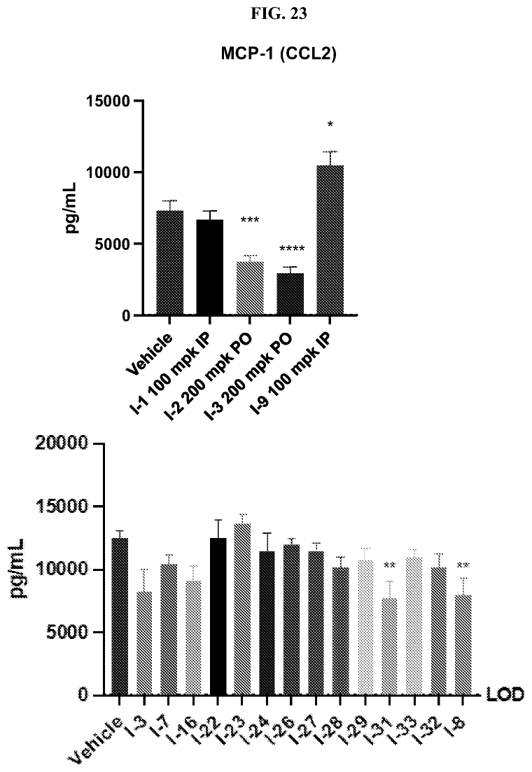

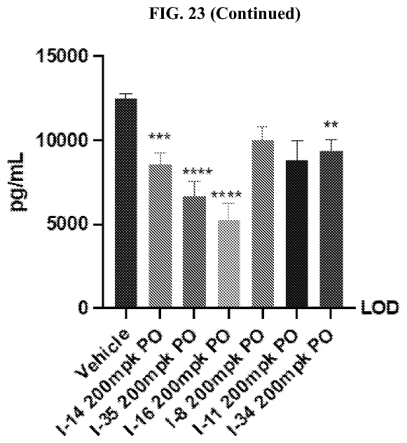

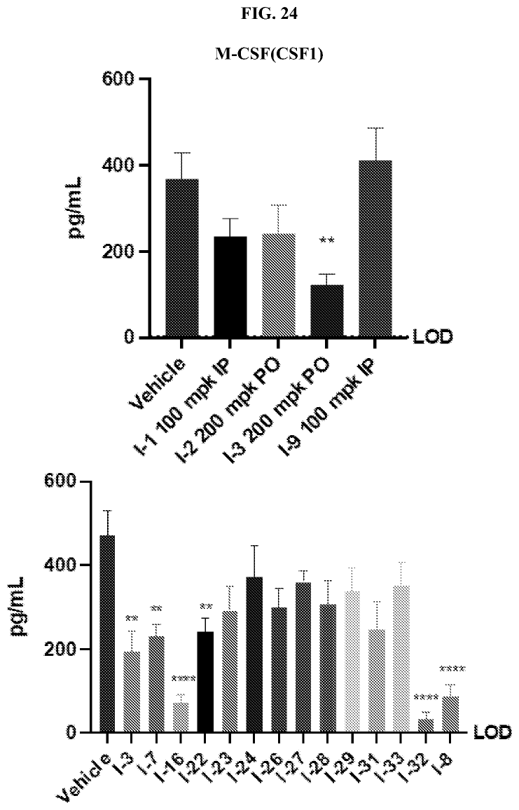

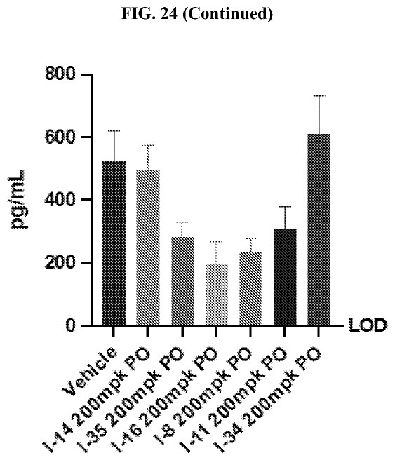

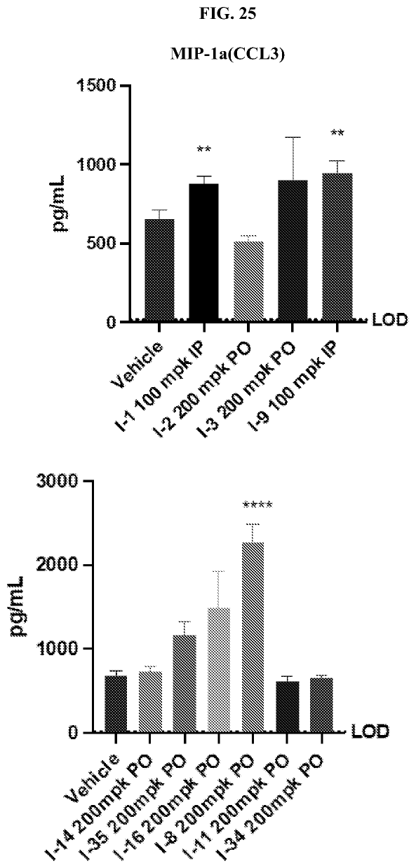

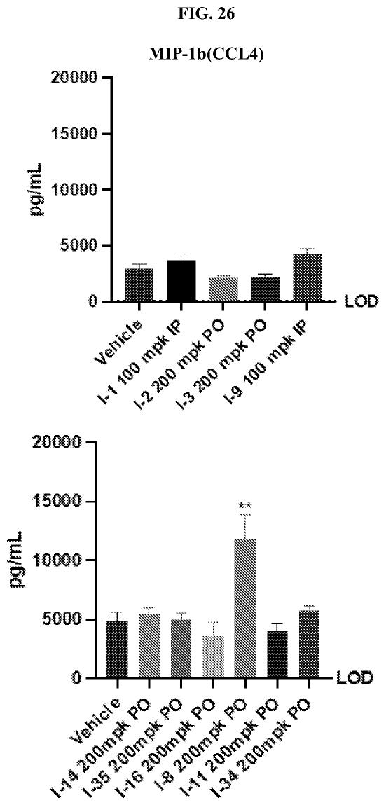

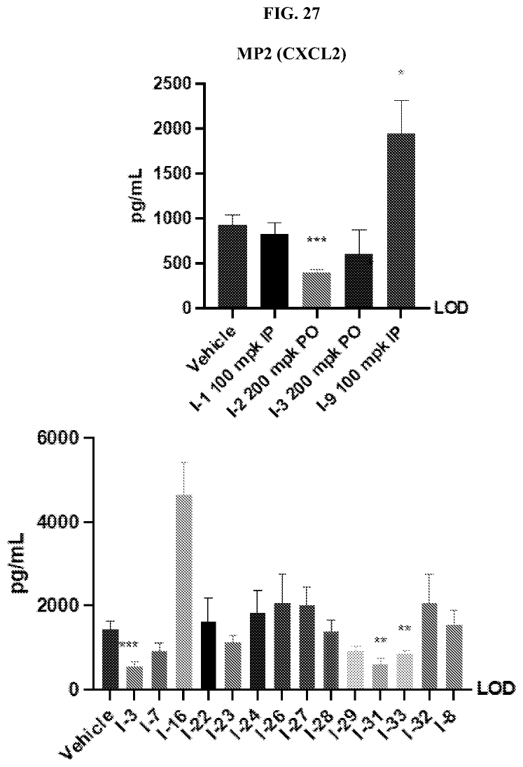

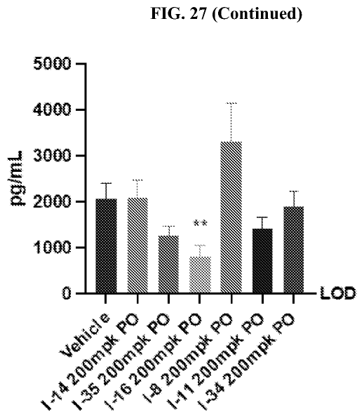

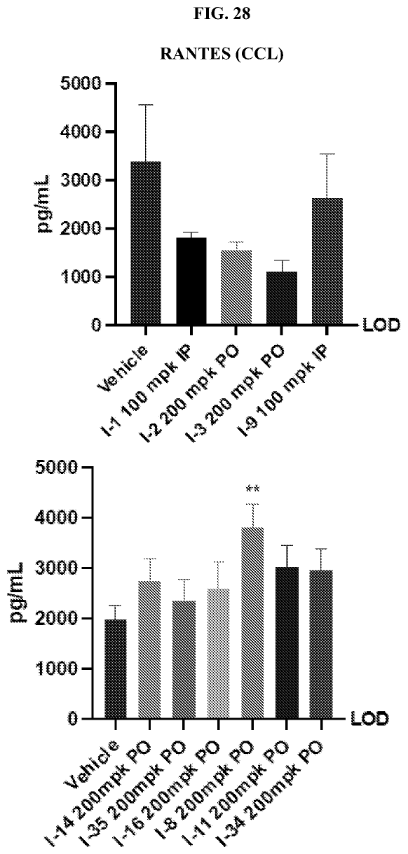

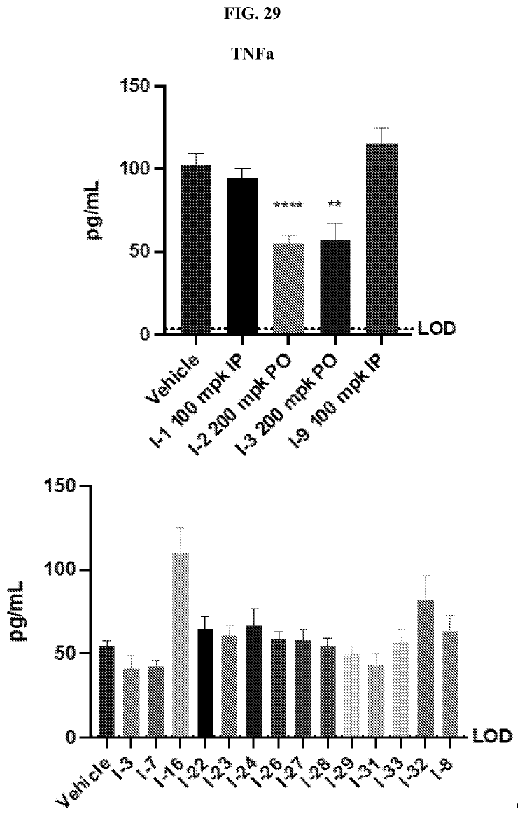

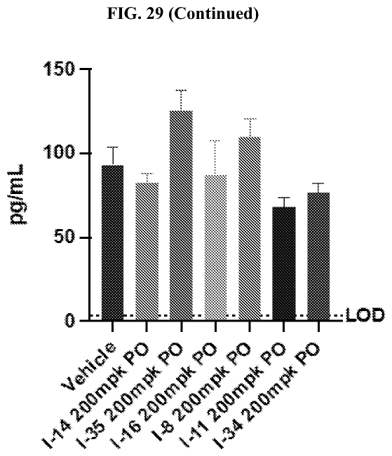

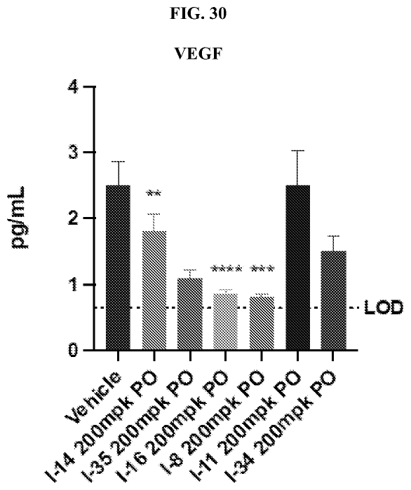

### DETAILED DESCRIPTION OF THE INVENTION

### 1. General Description of Certain Aspects of the Invention

In certain embodiments, the present invention provides compounds, compositions, and methods for treatment, prevention, and/or reduction of a risk of diseases, disorders, or conditions in which aldehyde toxicity is implicated in the pathogenesis. In some embodiments, such compounds include those of the formulae described herein, or pharmaceutically acceptable salts thereof, wherein each variable is as defined herein and described in embodiments. In some embodiments, a disclosed compound contains an amino functionality and a carbinol functionality (such as a propan-2-ol group) that are believed to be capable of scavenging or trapping aldehydes by formation of an adduct.

In one aspect, the present invention provides a compound of formula I:

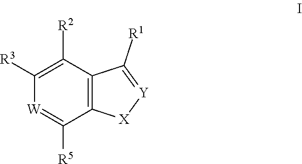

or a pharmaceutically acceptable salt thereof, wherein:
W is N or CR⁴;
X is S, NH, or O;
Y is N or CR⁶;
provided that, if X is S or O, then Y is CR⁶;
each of R¹, R², R³, R⁴, R⁵, and R⁶ is independently hydrogen, deuterium, halogen, —NH₂, —CN, —OR, —SR, optionally substituted C₁₋₆ aliphatic, or

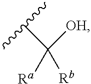

provided that one of R¹, R², R³, R⁴, R⁵, and R⁶ is —NH₂ and another one of R¹, R², R³, R⁴, R⁵, and R⁶ is

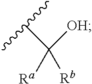

R^{a} is C₁₋₄ aliphatic optionally substituted with 1, 2, or 3 deuterium or halogen atoms; and R^{b} is C₁₋₄ aliphatic optionally substituted with 1, 2, or 3 deuterium or halogen atoms; or R^{a} and R^{b}, taken together with the carbon atom to which they are attached, form a 3-8 membered cycloalkyl or heterocyclyl ring containing 1-2 heteroatoms each independently selected from nitrogen, oxygen, and sulfur; and each R is independently selected from hydrogen, deuterium, and an optionally substituted group selected from: C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated monocyclic heterocyclic ring having 1-5 heteroatoms independently selected from nitrogen, oxygen, and sulfur; and a 8- to 10-membered bicyclic heteroaryl ring having 1-5 heteroatoms independently selected from nitrogen, oxygen, and sulfur.

In another aspect, the present invention provides a compound of formula VI:

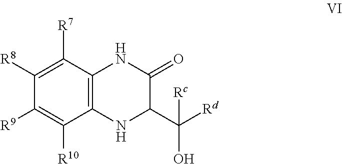

or a pharmaceutically acceptable salt thereof, wherein:
each of R⁷, R¹, R⁹, and R¹⁰ is independently hydrogen, deuterium, halogen, —N(R)₂, —CN, —OR, —SR, or optionally substituted C₁₋₆ aliphatic;
R^{c} is hydrogen or C₁₋₄ aliphatic optionally substituted with 1, 2, or 3 deuterium or halogen atoms;
R^{d} is hydrogen or C₁₋₄ aliphatic optionally substituted with 1, 2, or 3 deuterium or halogen atoms; or R^{c} and R^{d}, taken together with the carbon atom to which they are attached, form a 3-8 membered, saturated cycloalkyl or heterocyclyl ring containing 1-2 heteroatoms each independently selected from nitrogen, oxygen, and sulfur; and
   each R is independently selected from hydrogen, deuterium, and an optionally substituted group selected from: C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated monocyclic heterocyclic ring having 1-5 heteroatoms independently selected from nitrogen, oxygen, and sulfur; and a 8- to 10-membered bicyclic heteroaryl ring having 1-5 heteroatoms independently selected from nitrogen, oxygen, and sulfur.

### 2. Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

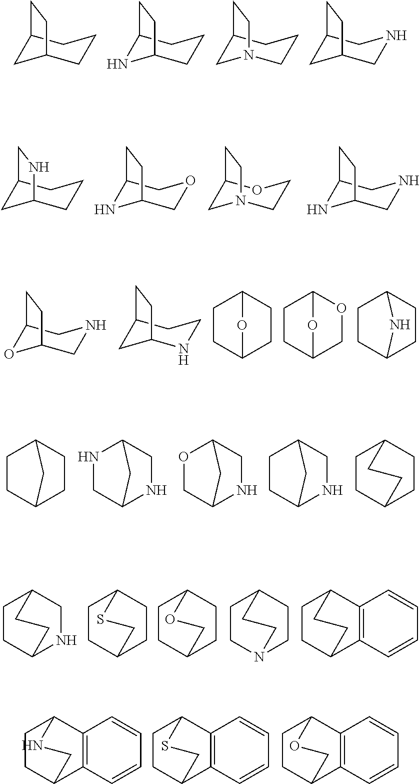

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}3; —(CH₂)₀₋₄OC(O)R^{◯}; —OC(O)(CH₂)₀₋₄SR—, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O)NR⁰²; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, mesylate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

The “retina” is a region of the central nervous system with approximately 150 million neurons. It is located at the back of the eye where it rests upon a specialized epithelial tissue called retinal pigment epithelium (RPE). The retina initiates the first stage of visual processing by transducing visual stimuli in specialized neurons called “photoreceptors”. Their synaptic outputs are processed by elaborate neural networks in the retina and are then transmitted to the brain. The retina has evolved two specialized classes of photoreceptors to operate under a wide range of light conditions. “Rod” photoreceptors transduce visual images under low light conditions and mediate achromatic vision. “Cone” photoreceptors transduce visual images in dim to bright light conditions and mediate both color vision and high acuity vision.

Every photoreceptor is compartmentalized into two regions called the “outer” and “inner” segment. The inner segment is the neuronal cell body containing the cell nucleus. The inner segment survives for a lifetime in the absence of retinal disease. The outer segment is the region where the light sensitive visual pigment molecules are concentrated in a dense array of stacked membrane structures. Part of the outer segment is routinely shed and regrown in a diurnal process called outer segment renewal. Shed outer segments are ingested and metabolized by RPE cells.

The “macula” is the central region of the retina which contains the fovea where visual images are processed by long slender cones in high spatial detail (“visual acuity”). “Macular degeneration” is a form of retinal neurodegeneration which attacks the macula and destroys high acuity vision in the center of the visual field. Age-Related Macular Degeneration (AMD) begins in a “dry form” characterized by residual lysosomal granules called lipofuscin in RPE cells, and by extracellular deposits called “drusen”. Drusen contain cellular waste products excreted by RPE cells. “Lipofuscin” and drusen can be detected clinically by ophthalmologists and quantified using fluorescence techniques. They can be the first clinical signs of macular degeneration.

Lipfuscin contains aggregations of A2E. Lipofuscin accumulates in RPE cells and poisons them by multiple known mechanisms. As RPE cells become poisoned, their biochemical activities decline and photoreceptors begin to degenerate. Extracellular drusen may further compromise RPE cells by interfering with their supply of vascular nutrients. Drusen also trigger inflammatory processes, which lead to choroidal neovascular invasions of the macula in one patient in ten who progresses to wet form AMD. Both the dry form and wet form progress to blindness.

“ERG” is an acronym for electroretinogram, which is the measurement of the electric field potential emitted by retinal neurons during their response to an experimentally defined light stimulus. ERG is a non-invasive measurement which can be performed on either living subjects (human or animal) or a hemisected eye in solution that has been removed surgically from a living animal.

As used herein, the term “RAL” means retinaldehyde. The term “RAL-trap” means a therapeutic compound that binds free RAL and thereby prevents the RAL from Schiff base condensation with membrane phosphatidylethanolamine (PE). “Free RAL” is defined as RAL that is not bound to a visual cycle protein. The terms “trans-RAL” and “all-trans-RAL” are used interchangeably and mean all trans-retinaldehyde.

A2E is a reaction by-product of a complex biochemical pathway called the “visual cycle” which operates collaboratively in both RPE cells and photoreceptor outer segments. The visual cycle recycles a photoreactive aldehyde chromophore called “retinaldehyde” which is derived from vitamin A and is essential for vision. In simplified terms, the visual cycle has four principal steps: 1) it converts vitamin A in the RPE into an aldehyde chromophore with one photoreactive strained double bond (11-cis-RAL); 2) it transports 11-cis-RAL to the retina where it binds to a specialized photoreceptor protein called opsin; 3) light photoisomerizes bound 11-cis-RAL to trans-RAL, which initiates the release of bound RAL from the opsin binding site; and 4) it converts trans-RAL (an aldehyde) to vitamin A (an alcohol) and transports vitamin A back to the RPE where the cycle begins again.

The aldehyde group of RAL helps bind the molecule to opsin by forming a reversible chemical bond to an amino acid sidechain in the opsin binding site. While the aldehyde group on RAL is essential for anchoring the molecule to the opsin binding site, it is otherwise hazardous because of its propensity to form Schiff bases with other biological amines. The first three reactions take place in photoreceptor outer segments and produce an intermediary product called A2PE. Once formed, A2PE partitions into the lipid phase and accumulates in photoreceptor outer segment membranes.

As described above, macular degeneration and other forms of retinal disease whose etiology involves the accumulation of A2E and/or lipofuscin may be treated or prevented by lowering the amount of A2E formed. Compounds useful for doing so include RAL-traps such as certain compounds disclosed herein. RAL-traps lower the amount of A2E formed, for example by forming a covalent bond with RAL that has escaped sequestering. RAL that has reacted with a RAL-trap compound is thereby unavailable to react with phosphatidylethanolamine.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

As used herein, “about” or “approximately” in reference to a numerical value means that the stated numerical value may vary by up to 10% of the stated value. For example, “about 10” refers to a value of 9.9 to 10.1 (10+/−0.1).

The term “biological sample,” as used herein, includes, without limitation, cell cultures or extracts thereof, biopsied material obtained from a mammal or extracts thereof, and blood, saliva, urine, feces, semen, tears, or other body fluids or extracts thereof.

### 3. Description of Exemplary Compounds

Compounds of the present invention, and compositions thereof, are useful for treating, preventing, and/or reducing a risk of a disease, disorder, or condition in which aldehyde toxicity is implicated in the pathogenesis.

According to one aspect, the present invention provides a compound of formula I:

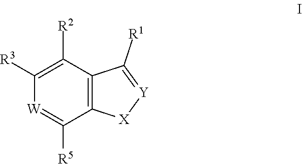

or a pharmaceutically acceptable salt thereof, wherein:
W is N or CR⁴;
X is S, NH, or O;
Y is N or CR⁶;
provided that, if X is S or O, then Y is CR⁶;
each of R¹, R², R³, R⁴, R⁵, and R⁶ is independently hydrogen, deuterium, halogen, —NH₂, —CN, —OR, —SR, optionally substituted C₁₋₆ aliphatic, or

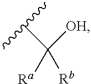

provided that one of R¹, R², R³, R⁴, R⁵, and R⁶ is —NH₂ and another one of R¹, R², R³, R⁴, R⁵, and R⁶ is

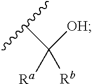

R^{a} is C₁₋₄ aliphatic optionally substituted with 1, 2, or 3 deuterium or halogen atoms; and
R^{b} is C₁₋₄ aliphatic optionally substituted with 1, 2, or 3 deuterium or halogen atoms; or R^{a} and R^{b}, taken together with the carbon atom to which they are attached, form a 3-8 membered cycloalkyl or heterocyclyl ring containing 1-2 heteroatoms each independently selected from nitrogen, oxygen, and sulfur; and
each R is independently selected from hydrogen, deuterium, and an optionally substituted group selected from: C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated monocyclic heterocyclic ring having 1-5 heteroatoms independently selected from nitrogen, oxygen, and sulfur; and a 8- to 10-membered bicyclic heteroaryl ring having 1-5 heteroatoms independently selected from nitrogen, oxygen, and sulfur.

In some embodiments of formula I, the —NH₂ that is one of R¹, R², R³, R⁴, R⁵, and R⁶ and the

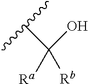

moiety that is another of R¹, R², R³, R⁴, R⁵, and R⁶ are on adjacent available carbon atoms. As a non-limiting example, if R¹ is —NH₂, then either R² or R⁶ is the carbinol moiety

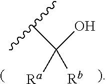

In some embodiments, one of R¹ and R⁶ is —NH₂ and one is

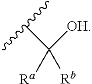

In some embodiments, one of R¹ and R² is —NH₂ and one is

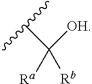

In some embodiments, one of R² and R³ is —NH₂ and one is

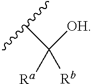

In some embodiments, one of R³ and R⁴ is —NH₂ and one is

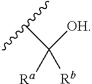

In some embodiments, one of R⁴ and R⁵ is —NH₂ and one is

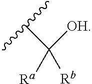

In some embodiments of formula I, one of R¹ and R⁶ is —NH₂ or

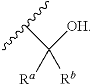

As defined generally above, W is N or CR⁴. In some embodiments, W is N. In some embodiments, W is CR⁴. In some embodiments, W is selected from those depicted in Table 1, below.

As defined generally above, X is S, NH or O. In some embodiments, X is S. In some embodiments, X is NH. In some embodiments, X is O. In some embodiments, X is selected from those depicted in Table 1, below.

As defined generally above, Y is N or CR⁶. In some embodiments, Y is N. In some embodiments, Y is CR⁶. In some embodiments, when X is NH, Y is N. In some embodiments, Y is selected from those depicted in Table 1, below.

In some embodiments, W is N, X is NH, and Y is N.

As defined generally above, R¹ is hydrogen, deuterium, halogen, —NH₂, —CN, —OR, —SR, optionally substituted C₁₋₆ aliphatic, or

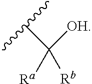

In some embodiments, R¹ is H. In some embodiments, R¹ is D. In some embodiments, R¹ is halogen. In some embodiments, R¹ is —NH₂. In some embodiments, R¹ is —CN. In some embodiments, R¹ is —OR. In some embodiments, R¹ is —SR. In some embodiments, R¹ is optionally substituted C₁₋₆ aliphatic. In some embodiments, R¹ is

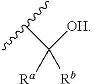

In some embodiments, R¹ is

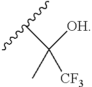 [SMILES: *C(C)(O)C(F)(F)F]

In some embodiments, R¹ is

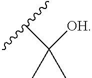 [SMILES: *C(C)(C)O]

In some embodiments, R¹ is selected from those depicted in Table 1, below.

As defined generally above, R² is hydrogen, deuterium, halogen, —NH₂, —CN, —OR, —SR, optionally substituted C₁₋₆ aliphatic, or

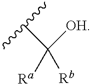

In some embodiments, R² is H. In some embodiments, R² is D. In some embodiments, R² is halogen. In some embodiments, R² is —NH₂. In some embodiments, R² is —CN. In some embodiments, R² is —OR. In some embodiments, R² is —SR. In some embodiments, R² is optionally substituted C₁₋₆ aliphatic. In some embodiments, R² is

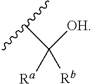

In some embodiments, R² is

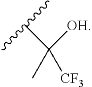 [SMILES: *C(C)(O)C(F)(F)F]

In some embodiments, R² is

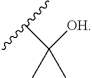 [SMILES: *C(C)(C)O]

In some embodiments, R² is Br. In some embodiments, R² is —CF₃. In some embodiments, R² is selected from those depicted in Table 1, below.

As defined generally above, R³ is hydrogen, deuterium, halogen, —NH₂, —CN, —OR, —SR, optionally substituted C₁₋₆ aliphatic, or

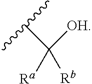

In some embodiments, R³ is H. In some embodiments, R³ is D. In some embodiments, R³ is halogen. In some embodiments, R³ is —NH₂. In some embodiments, R³ is —CN. In some embodiments, R³ is —OR. In some embodiments, R³ is —SR. In some embodiments, R³ is optionally substituted C₁₋₆ aliphatic. In some embodiments, R³ is

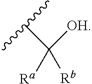

In some embodiments, R³ is

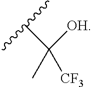 [SMILES: *C(C)(O)C(F)(F)F]

In some embodiments, R³ is

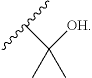 [SMILES: *C(C)(C)O]

In some embodiments, R³ is —CF₃. In some embodiments, R³ is selected from those depicted in Table 1, below.

As defined generally above, R⁴ is hydrogen, deuterium, halogen, —NH₂, —CN, —OR, —SR, optionally substituted C₁₋₆ aliphatic, or

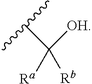

In some embodiments, R⁴ is H. In some embodiments, R⁴ is D. In some embodiments, R⁴ is halogen. In some embodiments, R⁴ is —NH₂. In some embodiments, R⁴ is —CN. In some embodiments, R⁴ is —OR. In some embodiments, R¹ is —SR. In some embodiments, R⁴ is optionally substituted C₁₋₆ aliphatic. In some embodiments, R⁴ is

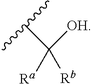

In some embodiments, R⁴ is

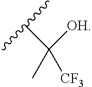 [SMILES: *C(C)(O)C(F)(F)F]

In some embodiments, R⁴ is

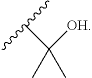 [SMILES: *C(C)(C)O]

In some embodiments, R⁴ is —CF₃. In some embodiments, R⁴ is selected from those depicted in Table 1, below.

As defined generally above, R⁵ is hydrogen, deuterium, halogen, —NH₂, —CN, —OR, —SR, optionally substituted C₁₋₆ aliphatic, or

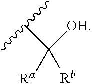

In some embodiments, R⁵ is H. In some embodiments, R⁵ is D. In some embodiments, R⁵ is halogen. In some embodiments, R⁵ is —NH₂. In some embodiments, R⁵ is —CN. In some embodiments, R⁵ is —OR. In some embodiments, R⁵ is —SR. In some embodiments, R⁵ is optionally substituted C₁₋₆ aliphatic. In some embodiments, R⁵ is

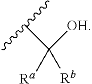

In some embodiments, R⁵ is

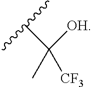 [SMILES: *C(C)(O)C(F)(F)F]

In some embodiments, R⁵ is

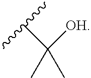 [SMILES: *C(C)(C)O]

In some embodiments, R⁵ is —CF₃. In some embodiments, R⁵ is selected from those depicted in Table 1, below.

As defined generally above, R⁶ is hydrogen, deuterium, halogen, —NH₂, —CN, —OR, —SR, optionally substituted C₁₋₆ aliphatic, or

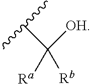

In some embodiments, R⁶ is H. In some embodiments, R⁶ is D. In some embodiments, R⁶ is halogen. In some embodiments, R⁶ is —NH₂. In some embodiments, R⁶ is —CN. In some embodiments, R⁶ is —OR. In some embodiments, R⁶ is —SR. In some embodiments, R⁶ is optionally substituted C₁₋₆ aliphatic. In some embodiments, R⁶ is

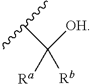

In some embodiments, R⁶ is

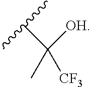 [SMILES: *C(C)(O)C(F)(F)F]

In some embodiments, R⁶ is

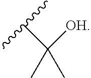 [SMILES: *C(C)(C)O]

In some embodiments, R⁶ s selected from those depicted in Table 1, below.

As defined generally above, R^{a} is C₁₋₄ aliphatic optionally substituted with 1, 2, or 3 deuterium or halogen atoms.

In some embodiments, R^{a} is C₁₋₄ aliphatic. In some embodiments, R^{a} is C₁₋₄ aliphatic optionally substituted with 1, 2, or 3 deuterium atoms. In some embodiments, R^{a} is C₁₋₄ aliphatic optionally substituted with 1, 2, or 3 halogen atoms.

In some embodiments, R^{a} is C₁₋₄ alkyl. In some embodiments, R^{a} is C₁₋₄ alkyl optionally substituted with 1, 2, or 3 deuterium or halogen atoms. In some embodiments, R^{a} is C₁₋₄ alkyl optionally substituted with 1, 2, or 3 deuterium atoms. In some embodiments, R^{a} is C₁₋₄ alkyl optionally substituted with 1, 2, or 3 halogen atoms. In some embodiments, R^{a} is methyl optionally substituted with 1, 2, or 3 halogen atoms. In some embodiments, R^{a} is —CF₃ or methyl.

As defined generally above, R^{b} is C₁₋₄ aliphatic optionally substituted with 1, 2, or 3 deuterium or halogen atoms.

In some embodiments, R^{b} is C₁₋₄ aliphatic. In some embodiments, R^{b} is C₁₋₄ aliphatic optionally substituted with 1, 2, or 3 deuterium atoms. In some embodiments, R^{b} is C₁₋₄ aliphatic optionally substituted with 1, 2, or 3 halogen atoms.

In some embodiments, R^{b} is C₁₋₄ alkyl. In some embodiments, R^{b} is C₁₋₄ alkyl optionally substituted with 1, 2, or 3 deuterium or halogen atoms. In some embodiments, R^{b} is C₁₋₄ alkyl optionally substituted with 1, 2, or 3 deuterium atoms. In some embodiments, R^{b} is C₁₋₄ alkyl optionally substituted with 1, 2, or 3 halogen atoms. In some embodiments, R^{b} is methyl optionally substituted with 1, 2, or 3 halogen atoms. In some embodiments, R^{b} is —CF₃ or methyl.

As generally defined above, R^{a} and R^{b} may be taken together with the carbon atom to which they are attached, form a 3-8 membered cycloalkyl or heterocyclyl ring containing 1-2 heteroatoms each independently selected from nitrogen, oxygen, and sulfur.

In some embodiments, R^{a} and R^{b}, taken together with the carbon atom to which they are attached, form a 3-8 membered cycloalkyl. In some embodiments, R^{a} and R^{b}, taken together with the carbon atom to which they are attached, form a 3-8 membered heterocyclyl ring containing 1-2 heteroatoms each independently selected from nitrogen, oxygen, and sulfur. In some embodiments, R^{a} and R^{b}, taken together with the carbon atom to which they are attached, form a cyclopropyl, cyclobutyl, or cyclopentyl ring. In some embodiments, R^{a} and R^{b}, taken together with the carbon atom to which they are attached, form an oxirane, oxetane, tetrahydrofuran, or aziridine.

In some embodiments, R^{a} and R^{b} are both methyl. In some embodiments, R^{a} is methyl and R^{b} is —CF₃. In some embodiments, R^{a} and R^{b} are selected from those depicted in Table 1, below.

In another aspect, the compound of formula I is a compound of formula II-a, II-b, II-c, II-d, II-e, II-f, II-g, II-h, IT-i, or II-j:

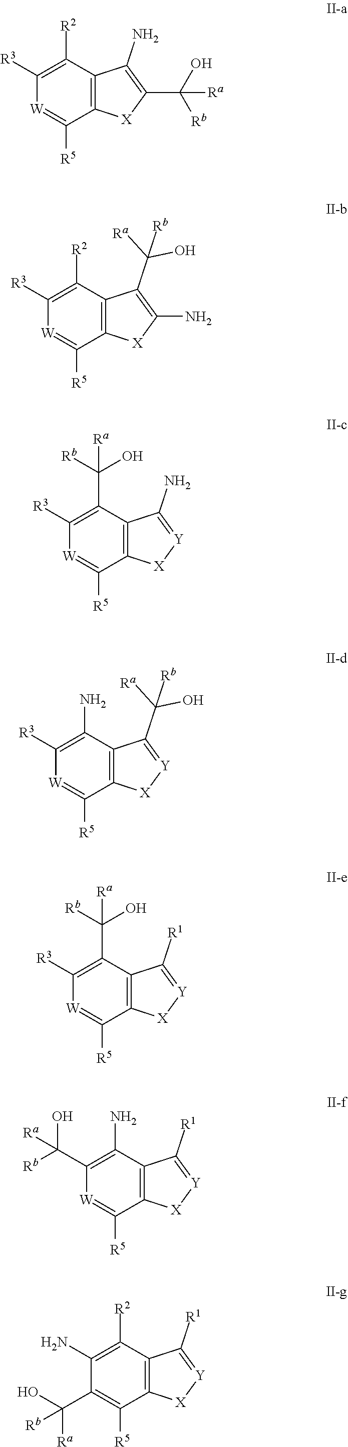

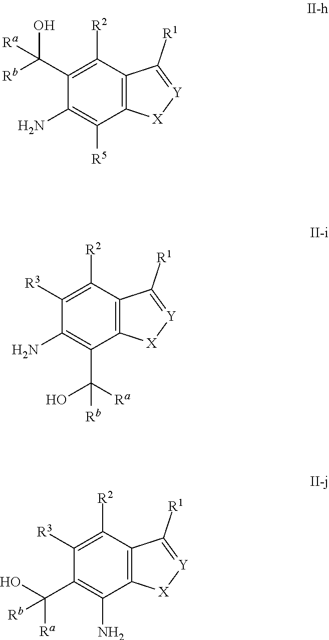

or a pharmaceutically acceptable salt thereof, wherein:
each of W, X, Y, R, R¹, R², R³, R⁴, R⁵, R⁶, R^{a}, and R^{b} is as defined above and described in embodiments herein, both singly and in combination.

In another aspect, the compound of formula I is a compound of formula III-a, III-b, III-c, III-d, III-e, III-f, III-g, III-h, ITT-i, or III-j:

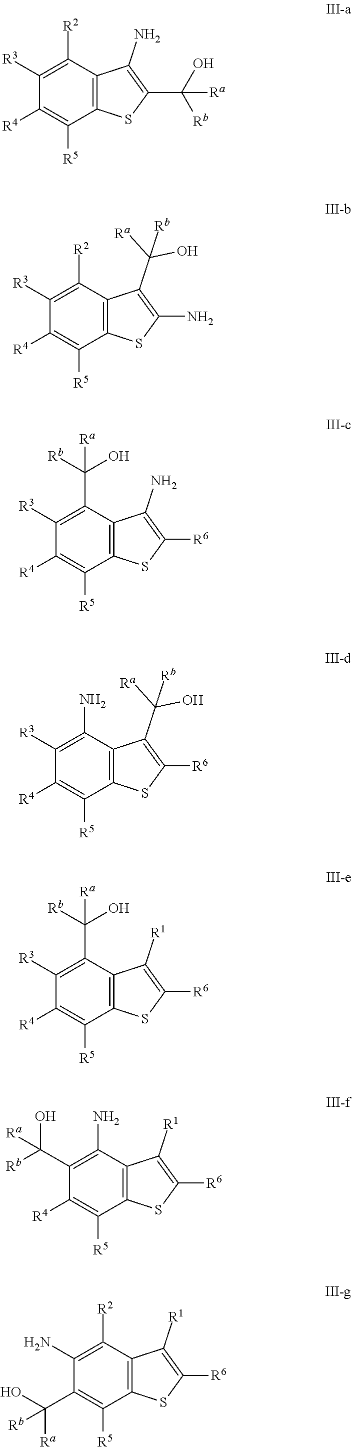

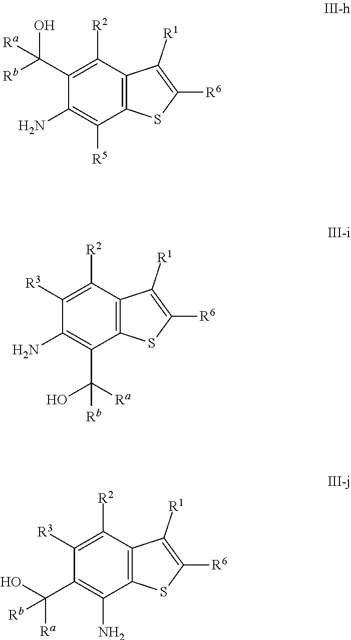

or a pharmaceutically acceptable salt thereof, wherein:
   each of R, R¹, R², R³, R⁴, R⁵, R⁶, R^{a}, and R^{b} is as defined above and described in embodiments herein, both singly and in combination.

In another aspect, the compound of formula I is a compound of formula IV-a, IV-b, IV-c, IV-d, IV-e, IV-f, IV-g, or IV-h:

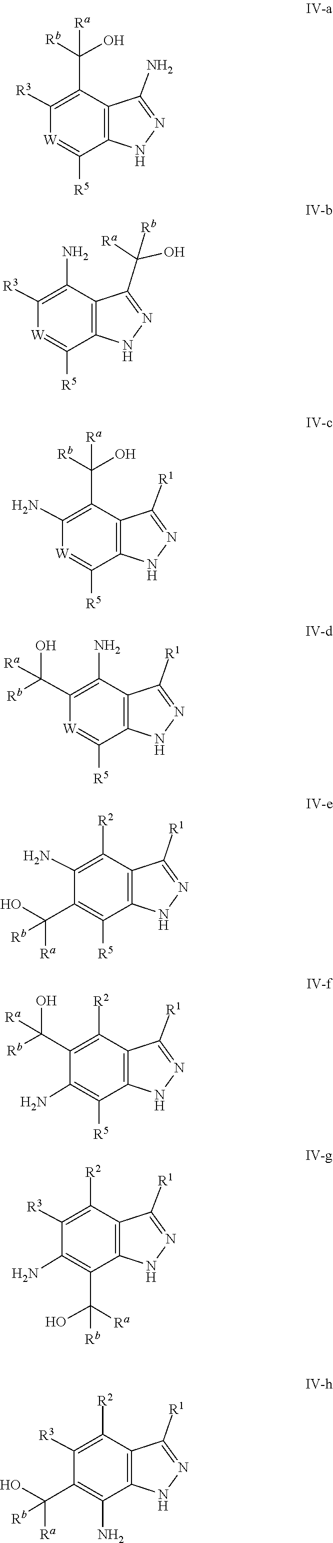

or a pharmaceutically acceptable salt thereof, wherein:
   each of W, R, R¹, R², R³, R⁴, R⁵, R⁶, R^{a} and R^{b} is as defined above and described in embodiments herein, both singly and in combination.

In another aspect, the compound of formula I is a compound of formula V-a, V-b, V-c, V-d, V-e, V-f, V-g, V-h, V-i, or V-j:

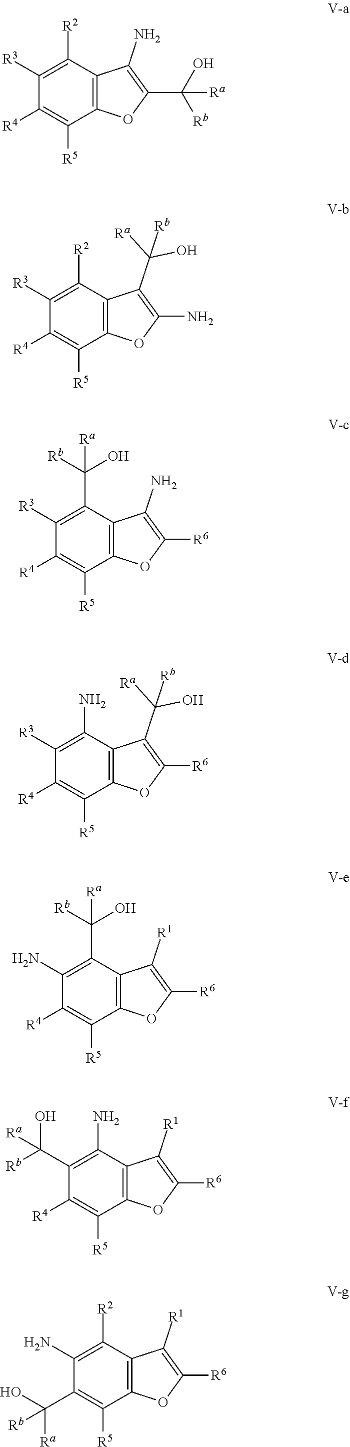

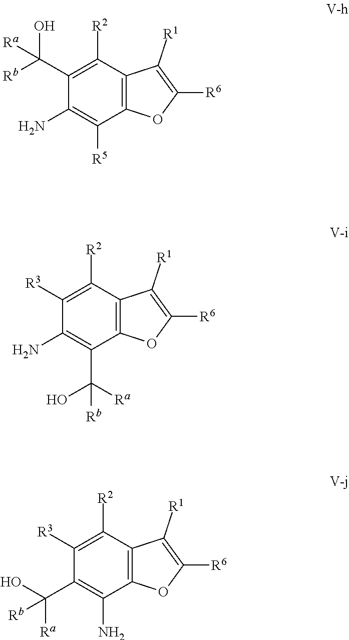

or a pharmaceutically acceptable salt thereof, wherein:
   each of R, R¹, R², R³, R⁴, R⁵, R⁶, R^{a}, and R^{b} is as defined above and described in embodiments herein, both singly and in combination.

In another aspect, the present invention provides a compound of formula VI:

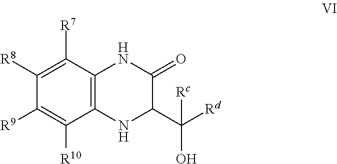

or a pharmaceutically acceptable salt thereof, wherein:
each of R⁷, R¹, R⁹, and R¹⁰ is independently hydrogen, deuterium, halogen, —N(R)₂, —CN, —OR, —SR, or optionally substituted C₁₋₆ aliphatic;
R^{c} is hydrogen or C₁₋₄ aliphatic optionally substituted with 1, 2, or 3 deuterium or halogen atoms;
R^{d} is hydrogen or C₁₋₄ aliphatic optionally substituted with 1, 2, or 3 deuterium or halogen atoms; or R^{c} and R^{d}, taken together with the carbon atom to which they are attached, form a 3-8 membered, saturated cycloalkyl or heterocyclyl ring containing 1-2 heteroatoms each independently selected from nitrogen, oxygen, and sulfur; and
each R is independently selected from hydrogen, deuterium, and an optionally substituted group selected from: C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated monocyclic heterocyclic ring having 1-5 heteroatoms independently selected from nitrogen, oxygen, and sulfur; and a 8- to 10-membered bicyclic heteroaryl ring having 1-5 heteroatoms independently selected from nitrogen, oxygen, and sulfur.

As defined generally above, R⁷ is hydrogen, deuterium, halogen, —N(R)₂, —CN, —OR, —SR, or optionally substituted C₁₋₆ aliphatic.

In some embodiments, R⁷ is H. In some embodiments, R⁷ is D. In some embodiments, R⁷ is halogen. In some embodiments, R⁷ is —N(R)₂. In some embodiments, R⁷ is —CN. In some embodiments, R⁷ is —OR. In some embodiments, R⁷ is —SR. In some embodiments, R⁷ is optionally substituted C₁₋₆ aliphatic. In some embodiments, R⁷ is —OMe. In some embodiments, R⁷ is —CF₃. In some embodiments, R⁷ is selected from those depicted in Table 1, below.

As defined generally above, R⁸ is hydrogen, deuterium, halogen, —N(R)₂, —CN, —OR, —SR, or optionally substituted C₁₋₆ aliphatic.

In some embodiments, R⁸ is H. In some embodiments, R⁸ is D. In some embodiments, R⁸ is halogen. In some embodiments, R⁸ is —N(R)₂. In some embodiments, R⁸ is —CN. In some embodiments, R⁸ is —OR. In some embodiments, R⁸ is —SR. In some embodiments, R⁸ is optionally substituted C₁₋₆ aliphatic. In some embodiments, R⁸ is —OMe. In some embodiments, R⁸ is —CF₃. In some embodiments, R⁸ is selected from those depicted in Table 1, below.

As defined generally above, R⁹ is hydrogen, deuterium, halogen, —N(R)₂, —CN, —OR, —SR, or optionally substituted C₁₋₆ aliphatic.

In some embodiments, R⁹ is H. In some embodiments, R⁹ is D. In some embodiments, R⁹ is halogen. In some embodiments, R⁹ is —N(R)₂. In some embodiments, R⁹ is —CN. In some embodiments, R⁹ is —OR. In some embodiments, R⁹ is —SR. In some embodiments, R⁹ is optionally substituted C₁₋₆ aliphatic. In some embodiments, R⁹ is —OMe. In some embodiments, R⁹ is —CF₃. In some embodiments, R⁹ is selected from those depicted in Table 1, below.

As defined generally above, R¹⁰ is hydrogen, deuterium, halogen, —N(R)₂, —CN, —OR, —SR, or optionally substituted C₁₋₆ aliphatic.

In some embodiments, R¹⁰ is H. In some embodiments, R¹⁰ is D. In some embodiments, R¹⁰ is halogen. In some embodiments, R¹⁰ is —N(R)₂. In some embodiments, R¹⁰ is —CN. In some embodiments, R¹⁰ is —OR. In some embodiments, R¹⁰ is —SR. In some embodiments, R¹⁰ is optionally substituted C₁₋₆ aliphatic. In some embodiments, R¹⁰ is —OMe. In some embodiments, R¹⁰ is —CF₃. In some embodiments, R¹⁰ is selected from those depicted in Table 1, below.

As defined generally above, R^{c} is hydrogen or C₁₋₄ aliphatic optionally substituted with 1, 2, or 3 deuterium or halogen atoms.

In some embodiments, R^{c} is hydrogen. In some embodiments, R^{c} is C₁₋₄ aliphatic. In some embodiments, R^{c} is C₁₋₄ aliphatic optionally substituted with 1, 2, or 3 deuterium atoms. In some embodiments, R^{a} is C₁₋₄ aliphatic optionally substituted with 1, 2, or 3 halogen atoms.

In some embodiments, R^{c} is C₁₋₄ alkyl. In some embodiments, R^{c} is C₁₋₄ alkyl optionally substituted with 1, 2, or 3 deuterium or halogen atoms. In some embodiments, R^{c} is C₁₋₄ alkyl optionally substituted with 1, 2, or 3 deuterium atoms. In some embodiments, R^{c} is C₁₋₄ alkyl optionally substituted with 1, 2, or 3 halogen atoms. In some embodiments, R^{c} is methyl optionally substituted with 1, 2, or 3 halogen atoms. In some embodiments, R^{c} is methyl.

As defined generally above, R^{d} is hydrogen or C₁₋₄ aliphatic optionally substituted with 1, 2, or 3 deuterium or halogen atoms.

In some embodiments, R^{d} is hydrogen. In some embodiments, R^{d} is C₁₋₄ aliphatic. In some embodiments, R^{d} is C₁₋₄ aliphatic optionally substituted with 1, 2, or 3 deuterium atoms. In some embodiments, R^{d} is C₁₋₄ aliphatic optionally substituted with 1, 2, or 3 halogen atoms.

In some embodiments, R^{d} is C₁₋₄ alkyl. In some embodiments, R^{d} is C₁₋₄ alkyl optionally substituted with 1, 2, or 3 deuterium or halogen atoms. In some embodiments, R^{d} is C₁₋₄ alkyl optionally substituted with 1, 2, or 3 deuterium atoms. In some embodiments, R^{d} is C₁₋₄ alkyl optionally substituted with 1, 2, or 3 halogen atoms. In some embodiments, R^{d} is methyl optionally substituted with 1, 2, or 3 halogen atoms. In some embodiments, R^{d} is methyl.

As defined generally above, R^{c} and R^{d}, taken together with the carbon atom to which they are attached, form a 3-8 membered, saturated cycloalkyl or heterocyclyl ring containing 1-2 heteroatoms each independently selected from nitrogen, oxygen, and sulfur.

In some embodiments, R^{c} and R^{d}, taken together with the carbon atom to which they are attached, form a 3-8 membered cycloalkyl. In some embodiments, R^{c} and R^{d}, taken together with the carbon atom to which they are attached, form a 3-8 membered heterocyclyl ring containing 1-2 heteroatoms each independently selected from nitrogen, oxygen, and sulfur. In some embodiments, R^{c} and R^{d}, taken together with the carbon atom to which they are attached, form a cyclopropyl, cyclobutyl, or cyclopentyl ring. In some embodiments, R^{c} and R^{d}, taken together with the carbon atom to which they are attached, form an oxirane, oxetane, tetrahydrofuran, or aziridine.

In some embodiments, R^{c} and R^{d} are both methyl. In some embodiments, R^{c} and R^{d} are both hydrogen. In some embodiments, R^{c} is methyl and R^{d} is —CF₃. In some embodiments, R^{c} and R^{d} are selected from those depicted in Table 1, below.

In some embodiments, the compound of formula VI is not

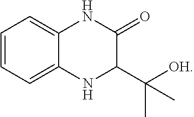

In some embodiments is not

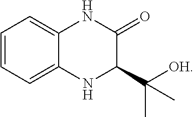

In some embodiments, the compound is not

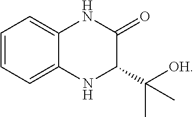

According to one aspect, the present invention provides a compound of formula VII:

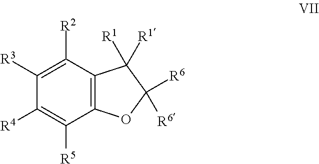

or a pharmaceutically acceptable salt thereof, wherein:
each of R¹, R², R³, R⁴, R⁵, and R⁶ is independently hydrogen, deuterium, halogen, —NH₂, —CN, —OR, —SR, —S(O)R, —S(O)₂R, optionally substituted C₁₋₆ aliphatic, or

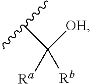

provided that one of R¹, R², R³, R⁴, R⁵, and R⁶ is —NH₂ and another one of R¹, R², R³, R⁴, R⁵, and R⁶ is

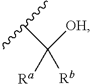

and the —NH₂ and the

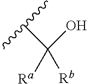

are attached to adjacent carbon atoms or have a peri relationship;
R^{1′} is hydrogen, deuterium, or C₁₋₆ alkyl;
R^{6′} is hydrogen, deuterium, or C₁₋₆ alkyl;
R^{a} is C₁₋₄ aliphatic optionally substituted with 1, 2, or 3 deuterium or halogen atoms; and
R^{b} is C₁₋₄ aliphatic optionally substituted with 1, 2, or 3 deuterium or halogen atoms; or R^{a} and R^{b}, taken together with the carbon atom to which they are attached, form a 3-8 membered cycloalkyl or heterocyclyl ring containing 1-2 heteroatoms each independently selected from nitrogen, oxygen, and sulfur; and
each R is independently selected from hydrogen, deuterium, and an optionally substituted group selected from: C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated monocyclic heterocyclic ring having 1-5 heteroatoms independently selected from nitrogen, oxygen, and sulfur; and a 8- to 10-membered bicyclic heteroaryl ring having 1-5 heteroatoms independently selected from nitrogen, oxygen, and sulfur.

In another aspect, the present invention provides a compound of formula VIII:

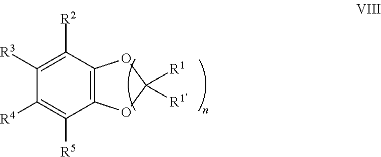

or a pharmaceutically acceptable salt thereof, wherein:
each of R², R³, R⁴, and R⁵ is independently hydrogen, deuterium, halogen, —NH₂, —CN, —OR, —SR, —S(O)R, —S(O)₂R, optionally substituted C₁₋₆ aliphatic, or

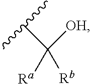

provided that one of R², R³, R⁴, and R⁵ is —NH₂ and another one of R², R³, R⁴, and R⁵ is

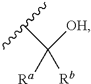

and the —NH₂ and the

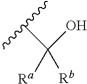

are attached to adjacent carbon atoms;
R¹ and R^{1′} are each independently hydrogen, deuterium, or C₁₋₆ alkyl;
R^{a} is C₁₋₄ aliphatic optionally substituted with 1, 2, or 3 deuterium or halogen atoms; and
R^{b} is C₁₋₄ aliphatic optionally substituted with 1, 2, or 3 deuterium or halogen atoms; or R^{a} and R^{b}, taken together with the carbon atom to which they are attached, form a 3-8 membered cycloalkyl or heterocyclyl ring containing 1-2 heteroatoms each independently selected from nitrogen, oxygen, and sulfur;
each R is independently selected from hydrogen, deuterium, and an optionally substituted group selected from: C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated monocyclic heterocyclic ring having 1-5 heteroatoms independently selected from nitrogen, oxygen, and sulfur; and a 8- to 10-membered bicyclic heteroaryl ring having 1-5 heteroatoms independently selected from nitrogen, oxygen, and sulfur; and
n is 1, 2, or 3.

As defined generally above, R¹ and R^{1′} are each independently hydrogen, deuterium, or C₁₋₆ alkyl.

In some embodiments, R¹ is H. In some embodiments, R¹ is D. In some embodiments, R¹ is C₁₋₆ alkyl.

In some embodiments, R¹ is selected from those depicted in Table 1, below.

In some embodiments, R^{1′} is H. In some embodiments, R^{1′} is D. In some embodiments, R^{1′} is C₁₋₆ alkyl.

In some embodiments, R^{1′} is selected from those depicted in Table 1, below.

As defined generally above, R² is hydrogen, deuterium, halogen, —NH₂, —CN, —OR, —SR, —S(O)R, —S(O)₂R, optionally substituted C₁₋₆ aliphatic, or

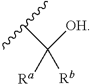

In some embodiments, R² is H. In some embodiments, R² is D. In some embodiments, R² is halogen. In some embodiments, R² is —NH₂. In some embodiments, R² is —CN. In some embodiments, R² is —OR. In some embodiments, R² is —SR. In some embodiments, R² is —S(O)R. In some embodiments, R² is —S(O)₂R. In some embodiments, R² is optionally substituted C₁₋₆ aliphatic. In some embodiments, R² is

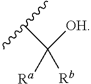

In some embodiments, R² is hydrogen, deuterium, halogen, —NH₂, —CN, —O(C₁₋₆ alkyl), —S(C₁₋₆ alkyl), —S(O)R, C₄₋₆ cycloakyl, C₁₋₆ alkyl, or

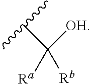

In some embodiments, R² is hydrogen, deuterium, halogen, —CN, —OMe, —S(C₁₋₆ alkyl), —S(O)(C₁₋₆ alkyl), or C₁₋₆ alkyl.

In some embodiments, R² is selected from those depicted in Table 1, below.

As defined generally above, R³ is hydrogen, deuterium, halogen, —NH₂, —CN, —OR, —SR, —S(O)R, —S(O)₂R, optionally substituted C₁₋₆ aliphatic, or

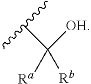

In some embodiments, R³ is H. In some embodiments, R³ is D. In some embodiments, R³ is halogen. In some embodiments, R³ is —NH₂. In some embodiments, R³ is —CN. In some embodiments, R³ is —OR. In some embodiments, R³ is —SR. In some embodiments, R³ is —S(O)R. In some embodiments, R³ is —S(O)₂R. In some embodiments, R³ is optionally substituted C₁₋₆ aliphatic. In some embodiments, R³ is

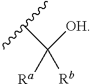

In some embodiments, R³ is hydrogen, deuterium, halogen, —NH₂, —CN, —O(C₁₋₆ alkyl), —S(C₁₋₆ alkyl), —S(O)R, C₄₋₆ cycloakyl, C₁₋₆ alkyl, or

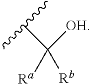

In some embodiments, R³ is hydrogen, deuterium, halogen, —CN, —OMe, —S(C₁₋₆ alkyl), —S(O)(C₁₋₆ alkyl), or C₁₋₆ alkyl.

In some embodiments, R³ is selected from those depicted in Table 1, below.

As defined generally above, R⁴ is hydrogen, deuterium, halogen, —NH₂, —CN, —OR, —SR, —S(O)R, —S(O)₂R, optionally substituted C₁₋₆ aliphatic, or

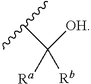

In some embodiments, R⁴ is H. In some embodiments, R⁴ is D. In some embodiments, R⁴ is halogen. In some embodiments, R⁴ is —NH₂. In some embodiments, R⁴ is —CN. In some embodiments, R⁴ is —OR. In some embodiments, R⁴ is —SR. In some embodiments, R⁴ is —S(O)R. In some embodiments, R⁴ is —S(O)₂R. In some embodiments, R⁴ is optionally substituted C₁₋₆ aliphatic. In some embodiments, R⁴ is

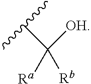

In some embodiments, R⁴ is hydrogen, deuterium, halogen, —NH₂, —CN, —O(C₁₋₆ alkyl), —S(C₁₋₆ alkyl), —S(O)R, C₄₋₆ cycloakyl, C₁₋₆ alkyl, or

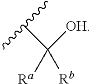

In some embodiments, R⁴ is hydrogen, deuterium, halogen, —CN, —OMe, —S(C₁₋₆ alkyl), —S(O)(C₁₋₆ alkyl), or C₁₋₆ alkyl.

In some embodiments, R⁴ is selected from those depicted in Table 1, below.

As defined generally above, R⁵ is hydrogen, deuterium, halogen, —NH₂, —CN, —OR, —SR, —S(O)R, —S(O)₂R, optionally substituted C₁₋₆ aliphatic, or

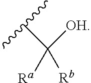

In some embodiments, R⁵ is H. In some embodiments, R⁵ is D. In some embodiments, R⁵ is halogen. In some embodiments, R⁵ is —NH₂. In some embodiments, R⁵ is —CN. In some embodiments, R⁵ is —OR. In some embodiments, R⁵ is —SR. In some embodiments, R⁵ is —S(O)R. In some embodiments, R⁵ is —S(O)₂R. In some embodiments, R⁵ is optionally substituted C₁₋₆ aliphatic. In some embodiments, R⁵ is

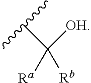

In some embodiments, R⁵ is hydrogen, deuterium, halogen, —NH₂, —CN, —O(C₁₋₆ alkyl), —S(C₁₋₆ alkyl), —S(O)R, C₄₋₆ cycloakyl, C₁₋₆ alkyl, or

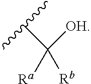

In some embodiments, R⁵ is hydrogen, deuterium, halogen, —CN, —OMe, —S(C₁₋₆ alkyl), —S(O)(C₁₋₆ alkyl), or C₁₋₆ alkyl.

In some embodiments, R⁵ is selected from those depicted in Table 1, below.

As defined generally above, n is 1, 2, or 3.

In some embodiments, n is 1. In some embodiments, n is 2. In some embodiments, n is 3.

In another aspect, the present invention provides a compound of formula IX:

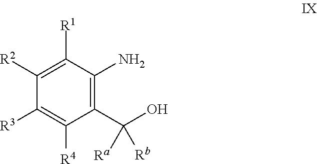

or a pharmaceutically acceptable salt thereof, wherein:
each of R¹, R², R³, and R⁴ is independently hydrogen, deuterium, halogen, —NH₂, —CN, —OR, —SR, —S(O)R, —S(O)₂R, or optionally substituted C₁₋₆ aliphatic;
R^{a} is C₁₋₄ aliphatic optionally substituted with 1, 2, or 3 deuterium or halogen atoms; and
R^{b} is C₁₋₄ aliphatic optionally substituted with 1, 2, or 3 deuterium or halogen atoms; or R^{a} and R^{b}, taken together with the carbon atom to which they are attached, form a 3-8 membered cycloalkyl or heterocyclyl ring containing 1-2 heteroatoms each independently selected from nitrogen, oxygen, and sulfur; and
each R is independently selected from hydrogen, deuterium, and an optionally substituted group selected from: C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated monocyclic heterocyclic ring having 1-5 heteroatoms independently selected from nitrogen, oxygen, and sulfur; and a 8- to 10-membered bicyclic heteroaryl ring having 1-5 heteroatoms independently selected from nitrogen, oxygen, and sulfur.

As defined generally above, R¹ is hydrogen, deuterium, halogen, —NH₂, —CN, —OR, —SR, —S(O)R, —S(O)₂R, or optionally substituted C₁₋₆ aliphatic.

In some embodiments, R¹ is H. In some embodiments, R¹ is D. In some embodiments, R¹ is halogen. In some embodiments, R¹ is —NH₂. In some embodiments, R¹ is —CN. In some embodiments, R¹ is —OR. In some embodiments, R¹ is —SR. In some embodiments, R¹ is —S(O)R. In some embodiments, R¹ is —S(O)₂R. In some embodiments, R¹ is optionally substituted C₁₋₆ aliphatic.

In some embodiments, R¹ is hydrogen, deuterium, halogen, —CN, —O(C₁₋₆ alkyl), —S(C₁₋₆ alkyl), —S(O)R, C₄₋₆ cycloakyl, or C₁₋₆ alkyl. In some embodiments, R¹ is hydrogen, deuterium, halogen, —CN, —OMe, —S(C₁₋₆ alkyl), —S(O)(C₁₋₆ alkyl), or C₁₋₆ alkyl.

In some embodiments, R¹ is selected from those depicted in Table 1, below.

As defined generally above, R² is hydrogen, deuterium, halogen, —NH₂, —CN, —OR, —SR, —S(O)R, —S(O)₂R, or optionally substituted C₁₋₆ aliphatic.

In some embodiments, R² is H. In some embodiments, R² is D. In some embodiments, R² is halogen. In some embodiments, R² is —NH₂. In some embodiments, R² is —CN. In some embodiments, R² is —OR. In some embodiments, R² is —SR. In some embodiments, R² is —S(O)R. In some embodiments, R² is —S(O)₂R. In some embodiments, R² is optionally substituted C₁₋₆ aliphatic.

In some embodiments, R² is hydrogen, deuterium, halogen, —CN, —O(C₁₋₆ alkyl), —S(C₁₋₆ alkyl), —S(O)R, C₄₋₆ cycloakyl, or C₁₋₆ alkyl. In some embodiments, R² is hydrogen, deuterium, halogen, —CN, —OMe, —S(C₁₋₆ alkyl), —S(O)(C₁₋₆ alkyl), or C₁₋₆ alkyl.

In some embodiments, R² is selected from those depicted in Table 1, below.

As defined generally above, R³ is hydrogen, deuterium, halogen, —NH₂, —CN, —OR, —SR, —S(O)R, —S(O)₂R, or optionally substituted C₁₋₆ aliphatic.

In some embodiments, R³ is H. In some embodiments, R³ is D. In some embodiments, R³ is halogen. In some embodiments, R³ is —NH₂. In some embodiments, R³ is —CN. In some embodiments, R³ is —OR. In some embodiments, R³ is —SR. In some embodiments, R³ is —S(O)R. In some embodiments, R³ is —S(O)₂R. In some embodiments, R³ is optionally substituted C₁₋₆ aliphatic.

In some embodiments, R³ is hydrogen, deuterium, halogen, —CN, —O(C₁₋₆ alkyl), —S(C₁₋₆ alkyl), —S(O)R, C₄₋₆ cycloakyl, or C₁₋₆ alkyl. In some embodiments, R³ is hydrogen, deuterium, halogen, —CN, —OMe, —S(C₁₋₆ alkyl), —S(O)(C₁₋₆ alkyl), or C₁₋₆ alkyl.

In some embodiments, R³ is selected from those depicted in Table 1, below.

As defined generally above, R⁴ is hydrogen, deuterium, halogen, —NH₂, —CN, —OR, —SR, —S(O)R, —S(O)₂R, or optionally substituted C₁₋₆ aliphatic.

In some embodiments, R⁴ is H. In some embodiments, R⁴ is D. In some embodiments, R⁴ is halogen. In some embodiments, R⁴ is —NH₂. In some embodiments, R⁴ is —CN. In some embodiments, R⁴ is —OR. In some embodiments, R⁴ is —SR. In some embodiments, R⁴ is —S(O)R. In some embodiments, R⁴ is —S(O)₂R. In some embodiments, R⁴ is optionally substituted C₁₋₆ aliphatic.

In some embodiments, R⁴ is hydrogen, deuterium, halogen, —CN, —O(C₁₋₆ alkyl), —S(C₁₋₆ alkyl), —S(O)R, C₄₋₆ cycloakyl, or C₁₋₆ alkyl. In some embodiments, R⁴ is hydrogen, deuterium, halogen, —CN, —OMe, —S(C₁₋₆ alkyl), —S(O)(C₁₋₆ alkyl), or C₁₋₆ alkyl.

In some embodiments, R⁴ is selected from those depicted in Table 1, below.

As defined generally above, R^{a} is C₁₋₄ aliphatic optionally substituted with 1, 2, or 3 deuterium or halogen atoms.

In some embodiments, R^{a} is C₁₋₄ aliphatic. In some embodiments, R^{a} is C₁₋₄ aliphatic optionally substituted with 1, 2, or 3 deuterium atoms. In some embodiments, R^{a} is C₁₋₄ aliphatic optionally substituted with 1, 2, or 3 halogen atoms.

In some embodiments, R^{a} is C₁₋₄ alkyl. In some embodiments, R^{a} is C₁₋₄ alkyl optionally substituted with 1, 2, or 3 deuterium or halogen atoms. In some embodiments, R^{a} is C₁₋₄ alkyl optionally substituted with 1, 2, or 3 deuterium atoms. In some embodiments, R^{a} is C₁₋₄ alkyl optionally substituted with 1, 2, or 3 halogen atoms. In some embodiments, R^{a} is methyl optionally substituted with 1, 2, or 3 halogen atoms. In some embodiments, R^{a} is —CF₃ or methyl.

As defined generally above, R^{b} is C₁₋₄ aliphatic optionally substituted with 1, 2, or 3 deuterium or halogen atoms.

In some embodiments, R^{b} is C₁₋₄ aliphatic. In some embodiments, R^{b} is C₁₋₄ aliphatic optionally substituted with 1, 2, or 3 deuterium atoms. In some embodiments, R^{b} is C₁₋₄ aliphatic optionally substituted with 1, 2, or 3 halogen atoms.

In some embodiments, R^{b} is C₁₋₄ alkyl. In some embodiments, R^{b} is C₁₋₄ alkyl optionally substituted with 1, 2, or 3 deuterium or halogen atoms. In some embodiments, R^{b} is C₁₋₄ alkyl optionally substituted with 1, 2, or 3 deuterium atoms. In some embodiments, R^{b} is C₁₋₄ alkyl optionally substituted with 1, 2, or 3 halogen atoms. In some embodiments, R^{b} is methyl optionally substituted with 1, 2, or 3 halogen atoms. In some embodiments, R^{b} is —CF₃ or methyl.

As generally defined above, R^{a} and R^{b} may be taken together with the carbon atom to which they are attached, form a 3-8 membered cycloalkyl or heterocyclyl ring containing 1-2 heteroatoms each independently selected from nitrogen, oxygen, and sulfur.

In some embodiments, R^{a} and R^{b}, taken together with the carbon atom to which they are attached, form a 3-8 membered cycloalkyl. In some embodiments, R^{a} and R^{b}, taken together with the carbon atom to which they are attached, form a 3-8 membered heterocyclyl ring containing 1-2 heteroatoms each independently selected from nitrogen, oxygen, and sulfur. In some embodiments, R^{a} and R^{b}, taken together with the carbon atom to which they are attached, form a cyclopropyl, cyclobutyl, or cyclopentyl ring. In some embodiments, R^{a} and R^{b}, taken together with the carbon atom to which they are attached, form an oxirane, oxetane, tetrahydrofuran, or aziridine.

In some embodiments, R^{a} and R^{b} are both methyl. In some embodiments, R^{a} is methyl and R^{b} is —CF₃. In some embodiments, R^{a} and R^{b} are selected from those depicted in Table 1, below.

In some embodiments,

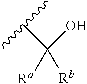

is

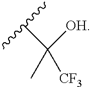 [SMILES: *C(C)(O)C(F)(F)F]

In some embodiments,

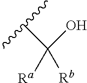

is

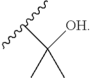 [SMILES: *C(C)(C)O]

In some embodiments, one, two, or three of R¹, R², R³, and R⁴ are not hydrogen. In some embodiments, one, two, or three of R¹, R², R³, and R⁴ are hydrogen.

In some embodiments, the compound of Formula IX is not

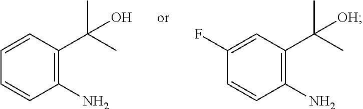

or a pharmaceutically acceptable salt thereof.

In some embodiments, the compound of Formula IX is

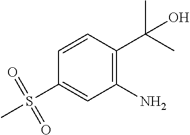

or a pharmaceutically acceptable salt thereof.

In another aspect, the present invention provides a compound of formula X:

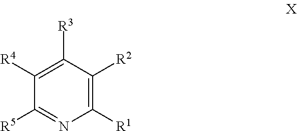

or a pharmaceutically acceptable salt thereof, wherein:
each of R¹, R², R³, R⁴, and R⁵ is independently hydrogen, deuterium, halogen, —NH₂, —CN, —OR, —SR, —S(O)R, —S(O)₂R, optionally substituted C₁₋₆ aliphatic, or

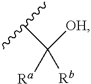

provided that one of R¹, R², R³, R⁴, and R⁵ is —NH₂ and another one of R¹, R², R³, R⁴, and R⁵ is

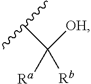

and the —NH₂ and the

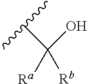

are attached to adjacent carbon atoms;
R^{a} is C₁₋₄ aliphatic optionally substituted with 1, 2, or 3 deuterium or halogen atoms; and
R^{b} is C₁₋₄ aliphatic optionally substituted with 1, 2, or 3 deuterium or halogen atoms; or R^{a} and R^{b}, taken together with the carbon atom to which they are attached, form a 3-8 membered cycloalkyl or heterocyclyl ring containing 1-2 heteroatoms each independently selected from nitrogen, oxygen, and sulfur; and
each R is independently selected from hydrogen, deuterium, and an optionally substituted group selected from: C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated monocyclic heterocyclic ring having 1-5 heteroatoms independently selected from nitrogen, oxygen, and sulfur; and a 8- to 10-membered bicyclic heteroaryl ring having 1-5 heteroatoms independently selected from nitrogen, oxygen, and sulfur.

As defined generally above, R¹ is hydrogen, deuterium, halogen, —NH₂, —CN, —OR, —SR, —S(O)R, —S(O)₂R, optionally substituted C₁₋₆ aliphatic, or

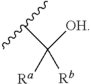

In some embodiments, R¹ is H. In some embodiments, R¹ is D. In some embodiments, R¹ is halogen. In some embodiments, R¹ is —NH₂. In some embodiments, R¹ is —CN. In some embodiments, R¹ is —OR. In some embodiments, R¹ is —SR. In some embodiments, R¹ is —S(O)R. In some embodiments, R¹ is —S(O)₂R. In some embodiments, R¹ is optionally substituted C₁₋₆ aliphatic. In some embodiments, R¹ is

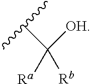

In some embodiments, R¹ is hydrogen, deuterium, halogen, —NH₂, —CN, —O(C₁₋₆ alkyl), —S(C₁₋₆ alkyl), —S(O)R, C₄₋₆ cycloakyl, C₁₋₆ alkyl, or

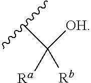

In some embodiments, R¹ is hydrogen, deuterium, halogen, —CN, —OMe, —S(C₁₋₆ alkyl), —S(O)(C₁₋₆ alkyl), or C₁₋₆ alkyl.

In some embodiments, R¹ is selected from those depicted in Table 1, below.

As defined generally above, R² is hydrogen, deuterium, halogen, —NH₂, —CN, —OR, —SR, —S(O)R, —S(O)₂R, optionally substituted C₁₋₆ aliphatic, or

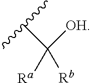

In some embodiments, R² is H. In some embodiments, R² is D. In some embodiments, R² is halogen. In some embodiments, R² is —NH₂. In some embodiments, R² is —CN. In some embodiments, R² is —OR. In some embodiments, R² is —SR. In some embodiments, R² is —S(O)R. In some embodiments, R² is —S(O)₂R. In some embodiments, R² is optionally substituted C₁₋₆ aliphatic. In some embodiments, R² is

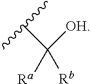

In some embodiments, R² is hydrogen, deuterium, halogen, —NH₂, —CN, —O(C₁₋₆ alkyl), —S(C₁₋₆ alkyl), —S(O)R, C₄₋₆ cycloakyl, C₁₋₆ alkyl, or

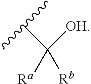

In some embodiments, R² is hydrogen, deuterium, halogen, —CN, —OMe, —S(C₁₋₆ alkyl), —S(O)(C₁₋₆ alkyl), or C₁₋₆ alkyl.

In some embodiments, R² is selected from those depicted in Table 1, below.

As defined generally above, R³ is hydrogen, deuterium, halogen, —NH₂, —CN, —OR, —SR, —S(O)R, —S(O)₂R, optionally substituted C₁₋₆ aliphatic, or

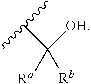

In some embodiments, R³ is H. In some embodiments, R³ is D. In some embodiments, R³ is halogen. In some embodiments, R³ is —NH₂. In some embodiments, R³ is —CN. In some embodiments, R³ is —OR. In some embodiments, R³ is —SR. In some embodiments, R³ is —S(O)R. In some embodiments, R³ is —S(O)₂R. In some embodiments, R³ is optionally substituted C₁₋₆ aliphatic. In some embodiments, R³ is

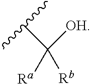

In some embodiments, R³ is hydrogen, deuterium, halogen, —NH₂, —CN, —O(C₁₋₆ alkyl), —S(C₁₋₆ alkyl), —S(O)R, C₄₋₆ cycloakyl, C₁₋₆ alkyl, or

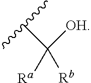

In some embodiments, R³ is hydrogen, deuterium, halogen, —CN, —OMe, —S(C₁₋₆ alkyl), —S(O)(C₁₋₆ alkyl), or C₁₋₆ alkyl.

In some embodiments, R³ is selected from those depicted in Table 1, below.

As defined generally above, R⁴ is hydrogen, deuterium, halogen, —NH₂, —CN, —OR, —SR, —S(O)R, —S(O)₂R, optionally substituted C₁₋₆ aliphatic, or

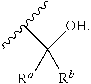

In some embodiments, R⁴ is H. In some embodiments, R⁴ is D. In some embodiments, R⁴ is halogen. In some embodiments, R⁴ is —NH₂. In some embodiments, R⁴ is —CN. In some embodiments, R⁴ is —OR. In some embodiments, R⁴ is —SR. In some embodiments, R⁴ is —S(O)R. In some embodiments, R⁴ is —S(O)₂R. In some embodiments, R⁴ is optionally substituted C₁₋₆ aliphatic. In some embodiments, R⁴ is

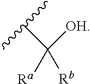

In some embodiments, R⁴ is hydrogen, deuterium, halogen, —NH₂, —CN, —O(C₁₋₆ alkyl), —S(C₁₋₆ alkyl), —S(O)R, C₄₋₆ cycloakyl, C₁₋₆ alkyl, or

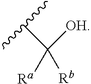

In some embodiments, R⁴ is hydrogen, deuterium, halogen, —CN, —OMe, —S(C₁₋₆ alkyl), —S(O)(C₁₋₆ alkyl), or C₁₋₆ alkyl.

In some embodiments, R⁴ is selected from those depicted in Table 1, below.

As defined generally above, R⁵ is hydrogen, deuterium, halogen, —NH₂, —CN, —OR, —SR, —S(O)R, —S(O)₂R, optionally substituted C₁₋₆ aliphatic, or

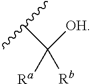

In some embodiments, R⁵ is H. In some embodiments, R⁵ is D. In some embodiments, R⁵ is halogen. In some embodiments, R⁵ is —NH₂. In some embodiments, R⁵ is —CN. In some embodiments, R⁵ is —OR. In some embodiments, R⁵ is —SR. In some embodiments, R⁵ is —S(O)R. In some embodiments, R⁵ is —S(O)₂R. In some embodiments, R⁵ is optionally substituted C₁₋₆ aliphatic. In some embodiments, R⁵ is

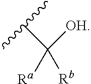

In some embodiments, R⁵ is hydrogen, deuterium, halogen, —NH₂, —CN, —O(C₁₋₆ alkyl), —S(C₁₋₆ alkyl), —S(O)R, C₄₋₆ cycloakyl, C₁₋₆ alkyl, or

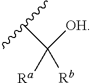

In some embodiments, R⁵ is hydrogen, deuterium, halogen, —CN, —OMe, —S(C₁₋₆ alkyl), —S(O)(C₁₋₆ alkyl), or C₁₋₆ alkyl.

In some embodiments, R⁵ is selected from those depicted in Table 1, below.

As defined generally above, R^{a} is C₁₋₄ aliphatic optionally substituted with 1, 2, or 3 deuterium or halogen atoms.

In some embodiments, R^{a} is C₁₋₄ aliphatic. In some embodiments, R^{a} is C₁₋₄ aliphatic optionally substituted with 1, 2, or 3 deuterium atoms. In some embodiments, R^{a} is C₁₋₄ aliphatic optionally substituted with 1, 2, or 3 halogen atoms.

In some embodiments, R^{a} is C₁₋₄ alkyl. In some embodiments, R^{a} is C₁₋₄ alkyl optionally substituted with 1, 2, or 3 deuterium or halogen atoms. In some embodiments, R^{a} is C₁₋₄ alkyl optionally substituted with 1, 2, or 3 deuterium atoms. In some embodiments, R^{a} is C₁₋₄ alkyl optionally substituted with 1, 2, or 3 halogen atoms. In some embodiments, R^{a} is methyl optionally substituted with 1, 2, or 3 halogen atoms. In some embodiments, R^{a} is —CF₃ or methyl.

As defined generally above, R^{b} is C₁₋₄ aliphatic optionally substituted with 1, 2, or 3 deuterium or halogen atoms.

In some embodiments, R^{b} is C₁₋₄ aliphatic. In some embodiments, R^{b} is C₁₋₄ aliphatic optionally substituted with 1, 2, or 3 deuterium atoms. In some embodiments, R^{b} is C₁₋₄ aliphatic optionally substituted with 1, 2, or 3 halogen atoms.

In some embodiments, R^{b} is C₁₋₄ alkyl. In some embodiments, R^{b} is C₁₋₄ alkyl optionally substituted with 1, 2, or 3 deuterium or halogen atoms. In some embodiments, R^{b} is C₁₋₄ alkyl optionally substituted with 1, 2, or 3 deuterium atoms. In some embodiments, R^{b} is C₁₋₄ alkyl optionally substituted with 1, 2, or 3 halogen atoms. In some embodiments, R^{b} is methyl optionally substituted with 1, 2, or 3 halogen atoms. In some embodiments, R^{b} is —CF₃ or methyl.

As generally defined above, R^{a} and R^{b} may be taken together with the carbon atom to which they are attached, form a 3-8 membered cycloalkyl or heterocyclyl ring containing 1-2 heteroatoms each independently selected from nitrogen, oxygen, and sulfur.

In some embodiments, R^{a} and R^{b}, taken together with the carbon atom to which they are attached, form a 3-8 membered cycloalkyl. In some embodiments, R^{a} and R^{b}, taken together with the carbon atom to which they are attached, form a 3-8 membered heterocyclyl ring containing 1-2 heteroatoms each independently selected from nitrogen, oxygen, and sulfur. In some embodiments, R^{a} and R^{b}, taken together with the carbon atom to which they are attached, form a cyclopropyl, cyclobutyl, or cyclopentyl ring. In some embodiments, R^{a} and R^{b}, taken together with the carbon atom to which they are attached, form an oxirane, oxetane, tetrahydrofuran, or aziridine.

In some embodiments, R^{a} and R^{b} are both methyl. In some embodiments, R^{a} is methyl and R^{b} is —CF₃. In some embodiments, R^{a} and R^{b} are selected from those depicted in Table 1, below.

In some embodiments

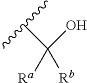

is

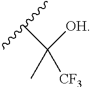 [SMILES: *C(C)(O)C(F)(F)F]

In some embodiments,

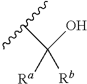

is

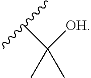 [SMILES: *C(C)(C)O]

In some embodiments, the compound of formula X is of formula X-a or X-b:

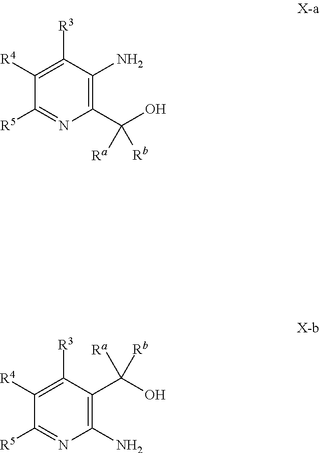

or a pharmaceutically acceptable salt thereof, wherein:
   each of R, R³, R⁴, R⁵, R^{a}, and R^{b} is as defined above and described in embodiments herein, both singly and in combination.

In another aspect, the present invention provides a compound selected from one of those depicted in Table 1, below.

TABLE 1
Representative Compounds

| Cmpd # | Structure |
|---|---|
| I-1 | 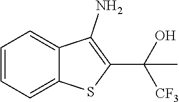 [SMILES: CC(O)(c1sc2ccccc2c1N)C(F)(F)F] |
| I-2 | 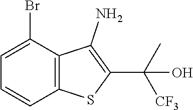 [SMILES: CC(O)(c1sc2cccc(Br)c2c1N)C(F)(F)F] |
| I-3 | 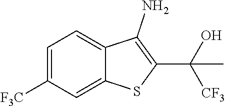 [SMILES: CC(O)(c1sc2cc(C(F)(F)F)ccc2c1N)C(F)(F)F] |
| I-3A | 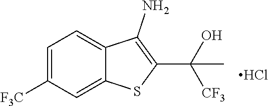 [SMILES: CC(O)(c1sc2cc(C(F)(F)F)ccc2c1N)C(F)(F)F ; Cl] |
| I-3B | 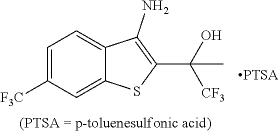 |
| I-4 | 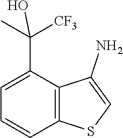 [SMILES: CC(O)(c1cccc2scc(N)c12)C(F)(F)F] |
| I-5 | 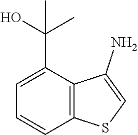 |
| I-6 | 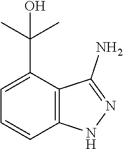 |
| I-7 | 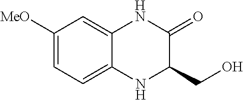 [SMILES: COc1ccc2c(c1)NC(=O)[C@@H](CO)N2] |
| I-8 | 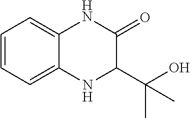 |
| I-9 | 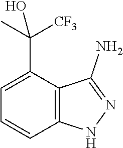 [SMILES: CC(O)(c1cccc2[nH]nc(N)c12)C(F)(F)F] |
| I-10 | 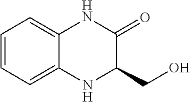 [SMILES: O=C1Nc2ccccc2N[C@@H]1CO] |
| I-11 | 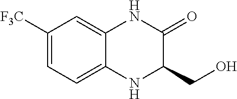 [SMILES: O=C1Nc2cc(C(F)(F)F)ccc2N[C@@H]1CO] |
| I-12 | 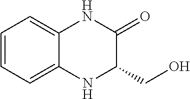 [SMILES: O=C1Nc2ccccc2N[C@H]1CO] |
| I-13 | 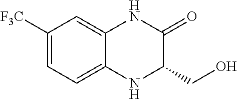 [SMILES: O=C1Nc2cc(C(F)(F)F)ccc2N[C@H]1CO] |
| I-14 | 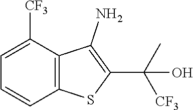 [SMILES: CC(O)(c1sc2cccc(C(F)(F)F)c2c1N)C(F)(F)F] |
| I-15 | 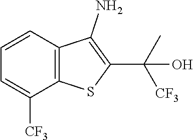 [SMILES: CC(O)(c1sc2c(C(F)(F)F)cccc2c1N)C(F)(F)F] |
| I-16 | 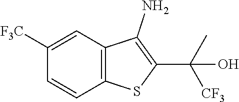 [SMILES: CC(O)(c1sc2ccc(C(F)(F)F)cc2c1N)C(F)(F)F] |
| I-17 | 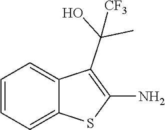 [SMILES: CC(O)(c1c(N)sc2ccccc12)C(F)(F)F] |
| I-18 | 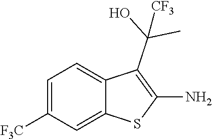 [SMILES: CC(O)(c1c(N)sc2cc(C(F)(F)F)ccc12)C(F)(F)F] |
| I-19 | 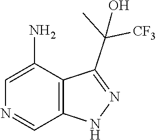 [SMILES: CC(O)(c1n[nH]c2cncc(N)c12)C(F)(F)F] |
| I-20 | 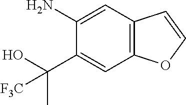 [SMILES: CC(O)(c1cc2occc2cc1N)C(F)(F)F] |
| I-21 | 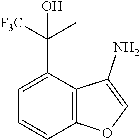 [SMILES: CC(O)(c1cccc2occ(N)c12)C(F)(F)F] |
| I-22 | 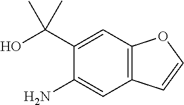 |
| I-23 | 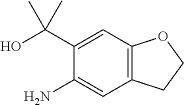 |
| I-24 | 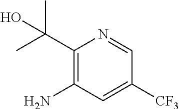 |
| I-25 | 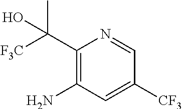 [SMILES: CC(O)(c1ncc(C(F)(F)F)cc1N)C(F)(F)F] |
| I-26 | 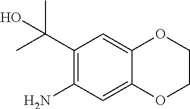 |
| I-27 | 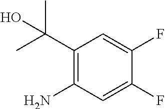 |
| I-28 | 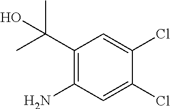 |
| I-29 | 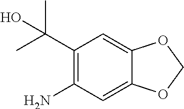 |
| I-30 | 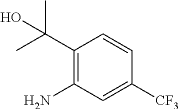 |
| I-31 | 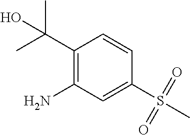 |
| I-32 | 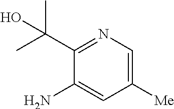 |
| I-33 | 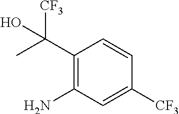 [SMILES: CC(O)(c1ccc(C(F)(F)F)cc1N)C(F)(F)F] |
| I-34 | 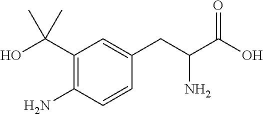 [SMILES: CC(C)(O)c1cc(CC(N)C(=O)O)ccc1N] |
| I-34A | 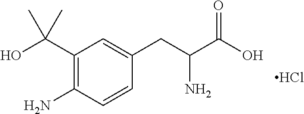 [SMILES: CC(C)(O)c1cc(CC(N)C(=O)O)ccc1N ; Cl] |
| I-35 | 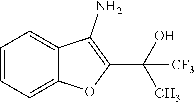 [SMILES: CC(O)(c1oc2ccccc2c1N)C(F)(F)F] |
| I-36 | 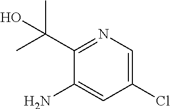 |
| I-37 | 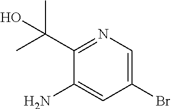 |

In some embodiments, the present invention provides a compound depicted in Table 1, above, or a pharmaceutically acceptable salt thereof.

In certain embodiments, the present invention provides any compound described above and herein, or a pharmaceutically acceptable salt thereof.

### 4. Uses of Compounds and Pharmaceutically Acceptable Compositions Thereof

Certain compounds described herein are found to be useful in scavenging toxic aldehydes, such as MDA and HNE. Without wishing to be bound by theory, it is believed that the compounds described herein undergo a Schiff base condensation with MDA, HNE, or other toxic aldehydes, and form a complex with the aldehydes in an energetically favorable reaction, thus reducing or eliminating aldehydes available for reaction with a protein, lipid, carbohydrate, or DNA. Importantly, compounds described herein can react with aldehydes to form a compound having a cyclic structure that contains the aldehydes, thus trapping the aldehydes and preventing the aldehydes from being released back into the cellular milieu.

In one aspect, the present invention provides a method for reducing levels of one or more toxic aldehydes in a subject, comprising administering to a subject in need thereof a disclosed compound or pharmaceutically acceptable salt thereof, or a pharmaceutical composition thereof, as described herein.

In another aspect, the present invention provides a method for reducing levels of one or more toxic aldehydes in a biological sample, comprising contacting the biological sample with a disclosed compound or pharmaceutically acceptable salt thereof, or a pharmaceutical composition thereof, as described herein. In some embodiments, the method is carried out in vitro.

In some embodiments, the toxic aldehyde is selected from formaldehyde, acetaldehyde, acrolein, glyoxal, methylglyoxal, hexadecanal, octadecanal, hexadecenal, succinic semi-aldehyde, malondialdehyde, 4-hydroxynonenal, 4-hydroxy-2E-hexenal, 4-hydroxy-2E,6Z-dodecadienal, retinaldehyde, leukotriene B4 aldehyde, and octadecenal.

In some embodiments, the toxic aldehyde is formaldehyde. In some embodiments, the toxic aldehyde is acetaldehyde. In some embodiments, the toxic aldehyde is acrolein. In some embodiments, the toxic aldehyde is glyoxal. In some embodiments, the toxic aldehyde is methylglyoxal. In some embodiments, the toxic aldehyde is hexadecanal. In some embodiments, the toxic aldehyde is octadecanal. In some embodiments, the toxic aldehyde is hexadecenal. In some embodiments, the toxic aldehyde is succinic semi-aldehyde (SSA). In some embodiments, the toxic aldehyde is malondialdehyde (MDA). In some embodiments, the toxic aldehyde is 4-hydroxynonenal. In some embodiments, the toxic aldehyde is retinaldehyde. In some embodiments, the toxic aldehyde is 4-hydroxy-2E-hexenal. In some embodiments, the toxic aldehyde is 4-hydroxy-2E,6Z-dodecadienal. In some embodiments, the aldehyde is leukotriene B4 aldehyde. In some embodiments, the aldehyde is octadecenal.

In some embodiments, the compound reduces systemic inflammation in the patient.

In some embodiments, the compound reduces plasma levels of a biomarker selected from IL-1β, IL-6, IL-10, and tumor necrosis factor alpha. In some embodiments, the compound reduces plasma levels of a biomarker selected from a RASP. In some embodiments, the RASP is malondialdehyde (MDA) and/or 4-hydroxynonenal (4-HNE).

In some embodiments, the method further comprises a reduction in the level of a reactive aldehyde species (RASP) in the patient's blood, such as malondialdehyde (MDA) or 4-hydroxynonenal (HNE).

In some embodiments, the level of RASP is reduced by at least 30%, at least 40%, or at least 50%. In some embodiments, the level of RASP is reduced by about 30% to 75%. In some embodiments, the level of RASP is reduced by about 20% to about 60%, or about 20% to about 50%, or about 20% to about 30%.

In another aspect, the present invention provides a method for treating a disease, disorder, or condition described herein, comprising administering to a subject in need thereof a disclosed compound or pharmaceutically acceptable salt thereof, or a pharmaceutical composition thereof, as described herein.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). In some embodiments, treatment is also continued after symptoms have resolved, for example to prevent, delay or lessen the severity of their recurrence.

The invention relates to compounds described herein for the treatment, prevention, and/or reduction of a risk of diseases, disorders, or conditions in which aldehyde toxicity is implicated in the pathogenesis.

Examples of the diseases, disorders, or conditions in which aldehyde toxicity is implicated include an ocular disease, disorder, or condition, including, but not limited to, a corneal disease (e.g., dry eye syndrome, cataracts, keratoconus, bullous and other keratopathy, and Fuch's endothelial dystrophy), other ocular disorders or conditions (e.g., allergic conjunctivitis, ocular cicatricial pemphigoid, conditions associated with PRK healing and other corneal healing, and conditions associated with tear lipid degradation or lacrimal gland dysfunction), and other ocular conditions associated with high aldehyde levels as a result of inflammation (e.g., uveitis, scleritis, ocular Stevens Johnson Syndrome, ocular rosacea (with or without meibomian gland dysfunction)). In one example, the ocular disease, disorder, or condition is not macular degeneration, such as age-related macular degeneration (“AMD”), or Stargardt's disease. In a further example, the ocular disease, disorder, or condition is dry eye syndrome, ocular rosacea, or uveitis.

Examples of the diseases, disorders, conditions, or indications in which aldehyde toxicity is implicated also include non-ocular disorders, including psoriasis, topical (discoid) lupus, contact dermatitis, atopic dermatitis, allergic dermatitis, radiation dermatitis, acne vulgaris, Sjogren-Larsson Syndrome and other ichthyosis, solar elastosis/wrinkles, skin tone firmness, puffiness, eczema, smoke or irritant induced skin changes, dermal incision, a skin condition associated burn and/or wound, lupus, scleroderma, asthma, chronic obstructive pulmonary disease (COPD), rheumatoid arthritis, inflammatory bowel disease, sepsis, atherosclerosis, ischemic-reperfusion injury, Parkinson's disease, Alzheimer's disease, succinic semialdehyde dehydrogenase deficiency (SSADHD), multiple sclerosis, amyotrophic lateral sclerosis, diabetes, metabolic syndrome, age-related disorders, and fibrotic diseases. In a further example, the non-ocular disorder is a skin disease, disorder, or condition selected from contact dermatitis, atopic dermatitis, allergic dermatitis, and. radiation dermatitis. In another example, the non-ocular disorder is a skin disease, disorder, or condition selected from Sjogren-Larsson Syndrome and a cosmetic indication associated with a burn and/or wound.

In a further example, the diseases, disorders, or conditions in which aldehyde toxicity is implicated are an age-related disorder. Examples of age-related diseases, disorders, or conditions include wrinkles, dryness, and pigmentation of the skin.

Examples of the diseases, disorders, or conditions in which aldehyde toxicity is implicated further include conditions associated with the toxic effects of blister agents or burns from alkali agents. The compounds described herein reduce or eliminate toxic aldehydes and thus treat, prevent, and/or reduce a risk of these diseases or disorders.

In some embodiments, the invention relates to the treatment, prevention, and/or reduction of a risk of an ocular disease, disorder, or condition in which aldehyde toxicity is implicated in the pathogenesis, comprising administering to a subject in need thereof a compound described herein. The ocular disease, disorder, or condition includes, but is not limited to, a corneal disease (e.g., dry eye syndrome, cataracts, keratoconus, bullous and other keratopathy, and Fuch's endothelial dystrophy in the cornea), other ocular disorders or conditions (e.g., allergic conjunctivitis, ocular cicatricial pemphigoid, conditions associated with PRK healing and other corneal healing, and conditions associated with tear lipid degradation or lacrimal gland dysfunction), and other ocular conditions where inflammation leads to high aldehyde levels (e.g., uveitis, scleritis, ocular Stevens Johnson Syndrome, ocular rosacea (with or without meibomian gland dysfunction)). In some embodiments, the ocular disease, disorder, or condition is macular degeneration. In some embodiments, the ocular disease, disorder, or condition is AMD or Stargardt's disease. In one illustration, in the ocular disease, disorder, or condition, the amount or concentration of MDA or HNE is increased in the ocular tissues or cells. For example, the amount or concentration of aldehydes (e.g., MDA or HNE) is increased for at least 1.1 fold, 1.2 fold, 1.3 fold, 1.4 fold, 1.5 fold, 2 fold, 2.5 fold, 5 fold, 10 fold as compared to that in normal ocular tissues or cells. Compounds described herein decrease aldehyde (e.g., MDA and/or HNE) concentration in a concentration-dependent manner. The amount or concentration of aldehydes (e.g., MDA or HNE) can be measured by methods or techniques known in the art, such as those described in Tukozkan et al., Furat Tip Dergisi 11: 88-92 (2006).

In some embodiments, the ocular disease, disorder, or condition is dry eye syndrome. In a second class, the ocular disease, disorder, or condition is a condition associated with PRK healing and other corneal healing. For example, the invention is directed to advancing PRK healing or other corneal healing, comprising administering to a subject in need thereof a compound described herein. In a third class, the ocular disease, disorder, or condition is an ocular condition associated with high aldehyde levels as a result of inflammation (e.g., uveitis, scleritis, ocular Stevens Johnson Syndrome, and ocular rosacea (with or without meibomian gland dysfunction). In a fourth class, the ocular disease, disorder, or condition is keratoconus, cataracts, bullous and other keratopathy, Fuchs' endothelial dystrophy, ocular cicatricial pemphigoid, or allergic conjunctivitis. The compound described herein may be administered topically or systemically, as described herein below.

In some embodiments, the invention relates to the treatment, prevention, and/or reduction of a risk of a skin disorder or condition or a cosmetic indication, in which aldehyde toxicity is implicated in the pathogenesis, comprising administering to a subject in need thereof a compound described herein. The skin disorder or condition includes, but is not limited to, psoriasis, scleroderma, topical (discoid) lupus, contact dermatitis, atopic dermatitis, allergic dermatitis, radiation dermatitis, acne vulgaris, and Sjogren-Larsson Syndrome and other ichthyosis, and the cosmetic indication is solar elastosis/wrinkles, skin tone firmness, puffiness, eczema, smoke or irritant induced skin changes, dermal incision, or a skin condition associated burn and/or wound. In some embodiments, the present invention relates to treatment of age-related diseases, disorders, or conditions of the skin, as described herein.

Various skin disorders or conditions, such as atopic dermatitis, topical (discoid) lupus, psoriasis and scleroderma, are characterized by high MDA and HNE levels (Br J Dermatol 149: 248 (2003); JEADV 26: 833 (2012); Clin Rheumatol 25: 320 (2006)). In addition, ichthyosis characteristic of the Sjogren-Larsson Syndrome (SLS) originates from accumulation of fatty aldehydes, which disrupts the normal function and secretion of lamellar bodies (LB) and leads to intercellular lipid deposits in the Strateum Corneum (SC) and a defective water barrier in the skin layer (W. B. Rizzo et al. (2010)). In patients with SLS, mutations in the gene encoding fatty aldehyde dehydrogenase, which metabolizes fatty aldehydes, significantly reduce or ablate its activity. Thus, compounds that reduce or eliminate aldehydes, such as the compounds described herein, can be used to treat, prevent, and/or reduction of a risk of skin disorders or conditions in which aldehyde toxicity is implicated in the pathogenesis, such as those described herein. Furthermore, with an improvement to the water barrier and prevention of aldehyde-mediated inflammation (including fibrosis and elastosis (Chairpotto et al. (2005)), many cosmetic indications, such as solar elastosis/wrinkles, skin tone, firmness (puffiness), eczema, smoke or irritant induced skin changes and dermal incision cosmesis, and skin conditions associated with a burn and/or wound can be treated using the method of the invention.

In some embodiments, the skin disease, disorder, or condition is psoriasis, scleroderma, topical (discoid) lupus, contact dermatitis, atopic dermatitis, allergic dermatitis, radiation dermatitis, acne vulgaris, or Sjogren-Larsson Syndrome and other ichthyosis. In one exemplification, the skin disease, disorder, or condition is contact dermatitis, atopic dermatitis, allergic dermatitis, radiation dermatitis, or Sjogren-Larsson Syndrome and other ichthyosis. In a second class, the cosmetic indication is solar elastosis/wrinkles, skin tone firmness, puffiness, eczema, smoke or irritant induced skin changes, dermal incision, or a skin condition associated with a burn and/or wound.

In some embodiments, the invention relates to the treatment, prevention, and/or reduction of a risk of a condition associated with the toxic effects of blister agents or burns from alkali agents in which aldehyde toxicity is implicated in the pathogenesis, comprising administering to a subject in need thereof a compound described herein.

Blister agents include, but are not limited to, sulfur mustard, nitrogen mustard, and phosgene oxime. Toxic or injurious effects of blister agents include pain, irritation, and/or tearing in the skin, eye, and/or mucous, and conjunctivitis and/or corneal damage to the eye. Sulfur mustard is the compound bis(2-chlorethyl) sulfide. Nitrogen mustard includes the compounds bis(2-chlorethyl)ethylamine, bis(2-chlorethyl)methylamine, and tris(2-chlorethyl)amine. Sulfur mustard or its analogs can cause an increase in oxidative stress and in particular in HNE levels, and by depleting the antioxidant defense system and thereby increasing lipid peroxidation, may induce an oxidative stress response and thus increase aldehyde levels (Jafari et al. (2010); Pal et al. (2009)). Antioxidants, such as silibinin, when applied topically, attenuate skin injury induced from exposure to sulfur mustard or its analogs, and increased activities of antioxidant enzymes may be a compensatory response to reactive oxygen species generated by the sulfur mustard (Jafari et al. (2010); Tewari-Singh et al. (2012)). Further, intervention to reduce free radical species was an effective treatment post exposure for phosgene induced lung injury (Sciuto et al. (2004)). Thus, compounds that reduce or eliminate aldehydes, such as compounds described herein, can be used to treat, prevent, and/or reduce a risk of a condition associated with the toxic effects of blister agents, such as sulfur mustard, nitrogen mustard, and phosgene oxime.

Alkali agents include, but are not limited to, lime, lye, ammonia, and drain cleaners. Compounds that reduce or eliminate aldehydes, such as compounds described herein, can be used to treat, prevent, and/or reduce a risk of a condition associated with burns from an alkali agent.

In some embodiments, the invention relates to the treatment, prevention, and/or reduction of a risk of an autoimmune, immune-mediated, inflammatory, cardiovascular, or neurological disease, disorder, or condition, or metabolic syndrome, or diabetes, in which aldehyde toxicity is implicated in the pathogenesis, comprising administering to a subject in need thereof a compound described herein. The autoimmune or immune-mediated disease, disorder, or condition includes, but is not limited to, lupus, scleroderma, asthma, chronic obstructive pulmonary disease (COPD), and rheumatoid arthritis. The inflammatory disease, disorder, or condition includes, but is not limited to, rheumatoid arthritis, inflammatory bowel disease (e.g., Crohn's disease and ulcerative colitis), sepsis, and fibrosis (e.g., renal, hepatic, pulmonary, and cardiac fibrosis). The cardiovascular disease, disorder, or condition includes, but is not limited to, atherosclerosis and ischemic-reperfusion injury. The neurological disease, disorder, or condition includes, but is not limited to, Parkinson's disease, Alzheimer's disease, succinic semialdehyde dehydrogenase deficiency, multiple sclerosis, amyotrophic lateral sclerosis, and the neurological aspects of Sjogren-Larsson Syndrome (cognitive delay and spasticity).

The present invention is also directed to the use of a compound described herein in the manufacture of a medicament for the treatment, prevention, and/or reduction of a risk of a disease, disorder, or condition in which aldehyde toxicity is implicated in the pathogenesis. More specifically, this aspect of the invention is directed to the use of a compound described herein in the manufacture of a medicament for the treatment, prevention, and/or reduction of a risk of (1) an ocular disease, disorder, or condition, including, but not limited to, a corneal disease (e.g., dry eye syndrome, cataracts, keratoconus, bullous and other keratopathy, and Fuch's endothelial dystrophy), other ocular disorders or conditions (e.g., allergic conjunctivitis, ocular cicatricial pemphigoid, conditions associated with PRK healing and other corneal healing, and conditions associated with tear lipid degradation or lacrimal gland dysfunction), and other ocular conditions associated with high aldehyde levels as a result of inflammation (e.g., uveitis, scleritis, ocular Stevens Johnson Syndrome, and ocular rosacea (with or without meibomian gland dysfunction)), (2) a skin disorder or condition or a cosmetic indication. For example, the disease, disorder, or condition includes, but is not limited to, psoriasis, topical (discoid) lupus, contact dermatitis, atopic dermatitis, allergic dermatitis, radiation dermatitis, acne vulgaris, Sjogren-Larsson Syndrome and other ichthyosis, solar elastosis/wrinkles, skin tone firmness, puffiness, eczema, smoke or irritant induced skin changes, dermal incision, and a skin condition associated with a burn and wound, (3) a condition associated with the toxic effects of blister agents or burns from alkali agents, or (4) an autoimmune, immune-mediated, inflammatory, cardiovascular, or neurological disease (e.g., lupus, scleroderma, asthma, chronic obstructive pulmonary disease (COPD), rheumatoid arthritis, inflammatory bowel disease, sepsis, atherosclerosis, ischemic-reperfusion injury, Parkinson's disease, Alzheimer's disease, multiple sclerosis, amyotrophic lateral sclerosis, diabetes, metabolic syndrome, and fibrotic diseases).

The present invention is also directed to the use of a compound described herein in treating, preventing, and/or reducing a risk of a disease, disorder, or condition in which aldehyde toxicity is implicated in the pathogenesis. More specifically, this aspect of the invention is directed to the use of a compound described herein in treating, preventing, and/or reducing a risk of (1) an ocular disease, disorder, or condition, including, but not limited to, a corneal disease (e.g., dry eye syndrome, cataracts, keratoconus, bullous and other keratopathy, and Fuch's endothelial dystrophy), other ocular disorders or conditions (e.g., allergic conjunctivitis, ocular cicatricial pemphigoid, conditions associated with PRK healing and other corneal healing, and conditions associated with tear lipid degradation or lacrimal gland dysfunction), and other ocular conditions associated with high aldehyde levels as a result of inflammation (e.g., uveitis, scleritis, ocular Stevens Johnson Syndrome, and ocular rosacea (with or without meibomian gland dysfunction)), (2) a skin disorder or condition or a cosmetic indication. For example, the disease, disorder, or condition includes, but is not limited to, psoriasis, topical (discoid) lupus, contact dermatitis, atopic dermatitis, allergic dermatitis, radiation dermatitis, acne vulgaris, Sjogren-Larsson Syndrome and other ichthyosis, solar elastosis/wrinkles, skin tone firmness, puffiness, eczema, smoke or irritant induced skin changes, dermal incision, and a skin condition associated with a burn and wound, (3) a condition associated with the toxic effects of blister agents or burns from alkali agents, or (4) an autoimmune, immune-mediated, inflammatory, cardiovascular, or neurological disease (e.g., lupus, scleroderma, asthma, chronic obstructive pulmonary disease (COPD), rheumatoid arthritis, inflammatory bowel disease, sepsis, atherosclerosis, ischemic-reperfusion injury, Parkinson's disease, Alzheimer's disease, multiple sclerosis, amyotrophic lateral sclerosis, diabetes, metabolic syndrome, and fibrotic diseases).

In some embodiments, the disease, disorder, or condition is a viral infection.

In some embodiments, the viral infection is caused by a coronavirus, hepatitis A virus, hepatitis B virus, dengue virus, yellow fever virus, Zika virus, influenza virus, respiratory syncytial virus (RSV), norovirus, herpesvirus, human immunodeficiency virus (HIV), Ebola virus, human T-lymphotropic virus (HTLV)-1 and -2, Epstein-Barr virus, Lassa virus, or Crimean-Congo hemorrhagic fever virus.

In some embodiments, the viral infection is caused by a coronavirus. In some embodiments, the coronavirus is an alpha, beta, gamma, or delta coronavirus. In some embodiments, the coronavirus is one associated with severe respiratory symptoms such as SARS.

In some embodiments, the viral infection is caused by a coronavirus, wherein the coronavirus is 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), or SARS-CoV-2 (coronavirus disease 2019, or COVID-19).

In some embodiments, the viral infection is caused by SARS-CoV-2.

In some embodiments, the viral infection is caused by an influenza virus.

In some embodiments, the viral infection is caused by an influenza virus, wherein the viral infection is selected from influenza type A and influenza type B. In some embodiments, the influenza virus is B/Yamagata or B/Victoria.

In some embodiments, the viral infection is caused by an influenza virus selected from H5N1, H1N1 and H3N2.

In some embodiments, the viral infection is caused by a Zika virus.

In some embodiments, a second therapeutic agent is administered to the patient, wherein the second therapeutic agent is selected from an antiviral agent, an antibiotic, and an NSAID.

In some embodiments, the second therapeutic agent is an antiviral agent, wherein the antiviral agent is appropriate for treating the viral infection.

In some embodiments, the second therapeutic agent is selected from chloroquine, remdesivir, hydroxychloroquine, interferon, ribavirin, umifenovir, teicoplanin, lopinavir, ritonavir, nitazoxanide, camostat, favipiravir, tocilizumab, and a passive antibody therapy.

In some embodiments, the present invention provides a method of treating, preventing, and/or reducing risk of a skin disease, disorder, or condition selected from atopic dermatitis, atopic eczema, and psoriasis, or an ocular disease, disorder, or condition selected from diabetic macular edema and Stargardt's disease, comprising administering to a patient in need thereof a disclosed pharmaceutical composition comprising a disclosed compound.

In some embodiments, the present disclosure provides use of the pharmaceutical composition described herein in the manufacture of a medicament for the treatment, prevention, and/or reduction of a risk of a skin disease, disorder or condition selected from atopic dermatitis, atopic eczema, and psoriasis, or an ocular disease, disorder or condition selected from diabetic macular edema and Stargardt's disease.

In some embodiments, a method of the disclosure is directed to treatment of atopic dermatitis. In some embodiments, a method of treating or reducing the risk of atopic dermatitis comprises administering to a patient in need thereof an effective amount of a compound disclosed herein. Generally, atopic dermatitis is characterized as an inflammatory condition of the skin presenting erythema, pruritus, scaling, lichenification, and papulovesicles. The pathogenesis of atopic dermatitis is multifactorial and involves a complex immunologic cascade, including disruption of the epidermal barrier, IgE dysregulation, defects in the cutaneous cell-mediated immune response, and genetic factors.

In some embodiments, the patient treated has a history of atopy (atopic disease). Generally, atopy refers to personal or family history of atopic eczema, asthma, and allergies.

In some embodiments, the atopic dermatitis treated is relapsing atopic dermatitis. Relapsing atopic dermatitis is a flare, exacerbation, or recurrence of atopic dermatitis following a remission of the disease, disorder or condition.

In some embodiments, the patient treated is identified as having a loss of function of profilaggrin (FLG) mutation. The inactive precursor profilaggrin protein is a large, complex, highly phosphorylated polypeptide that is the main constituent of the keratohyalin F granules that are visible in the granular cell layer of the epidermis. Various mutations in the FLG gene have been identified in individuals of atopic dermatitis and is a risk factor for atopic dermatitis (see, e.g., O'Regan et al., J Allergy Clinical Immunol., 2009; 124(3) Supplement 2:R2-R6).

In some embodiments, the patient has a loss of function of profilaggrin (FLG) mutation resulting in reduction of FLG protein expression.

In some embodiments, the atopic dermatitis treated is mild to moderate atopic dermatitis.

In some embodiments, the atopic dermatitis treated is moderate to severe atopic dermatitis.

In some embodiments, the atopic dermatitis treated is in the acute phase. In some embodiments, acute atopic dermatitis presents with a vesicular, weeping, crusting eruption.

In some embodiments, the atopic dermatitis treated is in the subacute phase. In some embodiments, subacute atopic dermatitis presents with dry, scaly, erythematous papules and plaques.

In some embodiments, the atopic dermatitis treated is in the chronic phase. In some embodiments, chronic atopic dermatitis demonstrates lichenification from repeated scratching.

In some embodiments, a method of the disclosure is directed to treatment of psoriasis. In some embodiments, a method of treating or reducing the risk of psoriasis comprises administering to a patient in need thereof an effective amount of a compound disclosed herein. Generally, psoriasis is a chronic, immune mediated disease characterized by raised, red, scaly patches on the skin. These conditions arise in part from acceleration of the growth cycle of skin cells.

In some embodiments, the psoriasis treated is plaque psoriasis. Plaque psoriasis usually presents as raised, inflamed, red lesions, covered by silvery, white scales, most often on the elbows, knees, scalp, and lower back.

In some embodiments, the psoriasis treated is guttate psoriasis. Guttate psoriasis often starts in childhood or young adulthood. It presents as small, red, individual spots on the skin, where the spots are not usually as thick or as crusty as the lesions in plaque psoriasis.

In some embodiments, the psoriasis treated is inverse psoriasis. Inverse psoriasis presents as red lesions, usually without the scales that occur in plaque psoriasis. The lesions can be smooth and shiny, and can occur in the armpits, groin, breast, and in skin folds.

In some embodiments, the psoriasis treated is pustular psoriasis. Pustular psoriasis presents as white pustules, or blisters of noninfectious pus, with red skin surrounds. It can affect certain areas of the body, such as the hands and feet, or most of the body.

In some embodiments, the psoriasis treated is erythrodermic psoriasis. Erythrodermic psoriasis is inflammatory and presents as exfoliation, or peeling of the skin with severe itching and pain. Edema may also be present.

In some embodiments, the psoriasis treated is mild psoriasis. Mild forms affect about 10% or less of total skin surface.

In some embodiments, the psoriasis treated is moderate to severe psoriasis. Moderate to severe forms affect >10% or more of total skin surface, and may require oral or systemic administration of therapeutic agents.

In some embodiments, the psoriasis treated is early onset psoriasis (type I psoriasis).

In some embodiments, the psoriasis treated is late onset psoriasis (type II psoriasis).

In some embodiments, a method of the disclosure is directed to treatment of diabetic macular edema (DME). In some embodiments, a method of treating or reducing the risk of diabetic macular edema (DME) comprises administering to a patient in need thereof an effective amount of a compound disclosed herein. Generally, DME is complication of diabetes and sometimes referred to a diabetic retinopathy. Damage to the small blood vessels of the retina arising from diabetes can result in leakage of fluid into the retina, which can lead to swelling of the surrounding tissue, including the macula, thereby leading to loss of vision.

In some embodiments, the patient treated is diagnosed with type 1 diabetes.

In some embodiments, the patient treated is diagnosed with type 2 diabetes.

In some embodiments, the DME treated is clinically significant macular edema (CSME). Clinically, CSME is defined as DME meeting at least one of the criteria presented as follows: (a) thickening of the retina at or within 500 μm of the center of the macula; (b) hard exudates at or within 500 μm of the center of the macula, if associated with thickening of adjacent retina (not counting residual hard exudates remaining after disappearance of retinal thickening); and (c) any zone(s) of retinal thickening 1 disc area or larger, any part of which is within 1 disc diameter of the center of the macula.

In some embodiments, the DME treated is center-involved DME. In center-involved DME, the central macula is generally the thickest portion of the retina and is an inversion of the normal morphology.

In some embodiments, the DME treated is non-center-involved DME. Non-central DME lack involvement of the center of the macula.

In some embodiments, the DME treated is focal DME. Focal edema often occurs associated with a cluster of microaneurysms, sometimes surrounded by an incomplete ring of hard exudates. It may be associated with less macular thickening, better visual acuity, and less severe retinopathy severity.

In some embodiments, the DME treated is diffuse type DME. Diffuse macular edema occurs from dilated retinal capillaries in the retina and involve a larger area of retinal thickening.

In some embodiments, the DME treated is accompanied by retinal detachment or severe non-clearing vitreous hemorrhage.

In some embodiments, the patient treated has undergone focal laser photocoagulation therapy.

In some embodiments, the patient treated has undergone grid laser photocoagulation therapy.

In some embodiments, a method of the disclosure is directed to treatment of Stargardt's disease. In some embodiments, a method of treating or reducing the risk of Stargardt's disease comprises administering to a patient in need thereof an effective amount of a compound disclosed herein. Generally, Stargardt's disease is inherited form of macular dystrophy characterized by bilateral vision loss, including dyschromatopsia and central scotomata, with characteristic macular atrophy and yellow-white flecks at the level of the retinal pigment epithelium (RPE) at the posterior pole. Stargardt's disease may also be referred to as Stargardt macular dystrophy, juvenile macular degeneration, or fundus flavimaculatus. Onset of Stargardt's disease occurs most commonly in childhood, with the next peak being early adulthood, and least frequently in later adulthood. Better prognosis is generally associated with a later onset.

In some embodiments, the Stargardt's disease treated is childhood-onset Stargardt's disease.

In some embodiments, the Stargardt's disease treated is adult-onset or late onset Stargardt's disease.

In some embodiments, the severity of Stargardt's disease can be classified based on electrophysiological assessement (see, e.g., Tanna et al., British Journal of Ophthalmology 2017; 101:25-30).

In some embodiments, the Stargardt's disease treated is classified in Group 1. Stargardt's disease in Group 1 display a severe pattern electroretinogram (ERG) abnormality (macular dysfunction) with normal full-field ERGs.

In some embodiments, the Stargardt's disease treated is classified in Group 2. Stargardt's disease in Group 2 display the characteristics of Group 1 and have in addition generalised loss of cone function. Patients in Group 2 have intermediate variable prognosis.

In some embodiments, the Stargardt's disease treated is classified in Group 3. Stargardt's disease in Group 3 display an additional generalised loss of both cone and rod function. Patients in Group 3 show the worst prognosis.

In some embodiments, the patient treated is identified as having a mutation in retina-specific ATP-binding cassette transporter (ABCA4) gene resulting in reduction or defect in ABCA4 function. Mutations in ABCA4 are the most common forms of inherited Stargardt's disease.

In some embodiments, the patient treated is identified as having mutation in ABCA4 gene that are associated with childhood-onset Stargardt's disease. Exemplary mutations associated with childhood-onset Stargardt's disease include, among others, 634C>T, 768G>T, 1317G>A, 1531C>T, 1557C>A, 5308T>G, 6088C>T, or 6449G>A.

In some embodiments, the patient treated is identified as having mutation in ABCA4 gene that are associated with adult-onset or late onset Stargardt's disease. Exemplary mutations associated with adult-onset or late onset Stargardt's disease include, among others, 769-784C>T, 2486C>T, 5603A>T, or 5882G>A.

As further discussed below, the compound or pharmaceutically acceptable salt thereof described herein can be administered systemically to treat the indications described herein. In some embodiments, the compound or pharmaceutically acceptable salt thereof is administered orally as part of a solid pharmaceutical composition. In some embodiments, the pharmaceutical composition is a liquid. In some embodiments, the pharmaceutical composition is administered as a liquid via nasogastric tube.

In some embodiments, for the ocular indications of diabetic macular edema or Stargardt's disease, the compound or pharmaceutically acceptable salt thereof is administered intravitreally.

In some embodiments, the disease, disorder, or condition is acute respiratory distress syndrome (ARDS). In some embodiments, the ARDS is associated with a viral infection. In some embodiments, the viral infection is accompanied by viral sepsis.

In some embodiments, the viral infection is accompanied by viral pneumonia.

In some embodiments, a method of treating ARDS comprises administering to a patient with ARDS resulting from or associated with a viral infection an effective amount of the pharmaceutical composition disclosed herein.

In some embodiments, the viral infection is caused by a coronavirus, hepatitis A virus, hepatitis B virus, dengue virus, yellow fever virus, Zika virus, influenza virus, norovirus, herpesvirus, respiratory syncytial virus (RSV), human immunodeficiency virus (HIV), Ebola virus, human T-lymphotropic virus (HTLV)-1 and -2, Epstein-Barr virus, Lassa virus, or Crimean-Congo hemorrhagic fever virus.

In some embodiments, the viral infection is caused by a coronavirus. In some embodiments, the viral infection is caused by a coronavirus selected from 229E, NL63, OC43, HKU1, MERS-CoV, SARS-CoV, and SARS-CoV-2.

In some embodiments, the viral infection is caused by a coronavirus. In some embodiments, the coronavirus is an alpha, beta, gamma, or delta coronavirus. In some embodiments, the coronavirus is one associated with severe respiratory symptoms such as SARS.

In some embodiments, the viral infection is caused by a coronavirus, wherein the coronavirus is 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), or SARS-CoV-2 (coronavirus disease 2019, or COVID-19).

In some embodiments, the viral infection is by a respiratory syncytial virus (RSV), influenza virus, coronavirus, or herpesvirus.

In some embodiments, the viral infection is by a coronavirus.

In some embodiments, the coronavirus is selected from 229E, NL63, OC43, HKU1, MERS-CoV, SARS-CoV, and SARS-CoV-2.

In some embodiments, the viral infection is by SARS-CoV-2.

In some embodiments, the viral infection is by an influenza virus.

In some embodiments, the influenza virus is influenza type A or influenza type B.

In some embodiments, the influenza virus is B/Yamagata or B/Victoria.

In some embodiments, the influenza virus is H5N1, H1N1 or H3N2.

In some embodiments, the ARDS is associated with a bacterial infection.

In some embodiments, the bacterial infection is accompanied by bacterial sepsis.

In some embodiments, the bacterial infection is accompanied by bacterial pneumonia.

In some embodiments, the bacterial infection is by Streptococcus pneumoniae, Staphylococcus aureus, Legionella pneumophila, Pneumocystis jirovecii, or Haemophilus influenza.

In some embodiments, the ARDS is associated with acute injury to the lungs caused by a chemical toxin or physical trauma.

In some embodiments, the acute injury to the lungs is by a chemical toxin.

In some embodiments, the chemical toxin that causes acute lung injury is a choking agent, vesicant, or nerve agent.

In some embodiments, the chemical toxin is a choking agent, wherein the choking agent is chlorine gas, phosgene, carbonyl chloride, hydrogen sulfide, or ammonia.

In some embodiments, the chemical toxin is a vesicant, wherein the vesicant is sulfur mustard or nitrogen mustard.

In some embodiments, the chemical toxin is a nerve agent, wherein the nerve agent is tabun, sarin, soman, or VX.

In some embodiments, the ARDS is associated with acute injury to the lungs caused by a biological toxin.

In some embodiments, the biological toxin is ricin, botulinum toxin, or staphylococcal enterotoxin B.

In some embodiments, the patient is being treated by mechanical ventilation.

In some embodiments, the disease, disorder, or condition is an inflammatory condition. In some embodiments, the inflammatory disorder is systemic. In some embodiments, the inflammatory disorder is localized to a particular tissue or organ. In some embodiments, the disease, disorder or condition for treatment with the compounds of the disclosure is non-alcoholic fatty liver disease (NAFLD), non-alcoholic steatohepatitis (NASH), inflammatory bowl disease, Crohn's disease, ulcerative colitis (UC), psoriasis, IBS (irritable bowel syndrome or spastic colon), including spastic colon, ankylosing spondylitis, osteoporosis, rheumatoid arthritis (RA), psoriatic arthritis, chronic obstructive pulmonary disease (COPD), atherosclerosis, pulmonary arterial hypertension, pyridoxine-dependent epilepsy, atopic dermatitis, atopic eczema, rosacea, multiple sclerosis (MS), systemic lupus erythematosus (SLE), lupus nephritis, sepsis, eosinophilic esophagitis, chronic kidney disease (CKD), fibrotic renal disease, chronic eosinophilic pneumonia, extrinsic allergic alveolitis, pre-eclampsia, endometriosis, polycystic ovary syndrome (PCOS), reduced female fertility, reduced sperm viability and motility, or cyclophosphamide-induced hemorrhagic cystitis.

In some embodiments, the disease, disorder, or condition for treatment with the compounds of the disclosure is lung-based chronic obstructive pulmonary disease (COPD), interstitial lung disease (ILD), idiopathic pulmonary fibrosis (IPF), cystic fibrosis (CF), emphysema due to alpha-1 antitrypsin deficiency, or pulmonary arterial hypertension (PAH).

In some embodiments, the disease, disorder, or condition for treatment with the compounds of the disclosure is light chain deposition disease, IgA nephropathy, end stage renal disease, gout, pseudogout, diabetic nephrophathy, diabetic neuropathy, traumatic brain injury, noise-induced hearing loss, Alzheimer's Disease, Parkinson's Disease, Huntington Disease, amyotrophic lateral sclerosis, primary biliary cirrhosis, primary sclerosing cholangitis, uterine leiomyoma, sarcoidosis, or chronic kidney disease.

In some embodiments, the disease, disorder, or condition for treatment with the compounds of the disclosure is an ocular inflammatory disorder. In some embodiments, the ocular inflammatory disorder is diabetic macular edema (DME), atopic keratoconjunctivitis (AKC), vernal keratoconjunctivitis (VKC), age-related macular degeneration (AMD), dry eye disease (DED), allergic conjunctivitis (AC), dry eye disease with allergic conjunctivitis, noninfectious anterior uveitis, posterior uveitis, pan-uveitis, post-surgical ocular pain and inflammation.

In some embodiments, the disease, disorder, or condition is one of those described in WO 2019/075136, which is hereby incorporated by reference.

In some embodiments, the compound or pharmaceutically acceptable salt thereof is administered once, twice, thrice, or four times per day. In some embodiments, the compound or pharmaceutically acceptable salt thereof is administered twice per day.

In some embodiments, the dose of the compound or pharmaceutically acceptable salt thereof is administered systemically.

In some embodiments, the dose of the compound or pharmaceutically acceptable salt thereof is administered orally.

In some embodiments, the pharmaceutical composition is a liquid. In some embodiments, the pharmaceutical composition is administered as a liquid via nasogastric tube.

In some embodiments, the method further comprises administering to the patient an effective amount of a second therapeutic agent suitable for treating ARDS.

In some embodiments, the second therapeutic agent is an anti-inflammatory agent selected from a steroid, anti-GM-CSF antibody, and a lung surfactant.

Chronic cough, pneumonia, and pulmonary sepsis are clinically distinct respiratory diseases, disorders, or conditions. Chronic cough is generally defined as cough lasting longer than 8 weeks and excluding cough with an underlying fever, such as from a bacterial or viral infection; chronic obstructive pulmonary disease (COPD) and other non-asthmatic pulmonary diseases; cancer of the lung or esophagus; pneumonia; interstitial lung disease; and obstructive sleep apnea. Pneumonia is an infection of the lungs by a pathogen, such as a bacteria, virus, or fungi. It is distinguished from Acute Respiratory Distress Syndrome, which can be caused by acute injury to the lung unrelated to infection by a pathogen. Pneumonia is usually diagnosed by a combination of clinical history, physical examination and/or laboratory tests, and clinical diagnosis from a chest X-ray (CXR), which can distinguish pneumonia from other respiratory tract infections. Pulmonary sepsis also affects the lungs but can arise from sepsis due to the sensitivity of the lungs and because sepsis can develop from infection of the lungs by a pathogen.

Alcohol induced hepatitis, minimal change disease, and focal segmental glomerulosclerosis affect the liver or kidneys rather than the lungs. Alcohol induced hepatitis is attributed to chronic abuse of alcohol, and is characterized by injury to the liver. Defining characteristics include hyperbilirubinemia and levels of liver function markers aspartate aminotransferase (AST) and alanine aminotransferase (ALT). Minimal change disease and focal segmental glomerulosclerosis are diseases, disorders, or conditions affecting the kidney. Both minimal change disease and focal segmental glomerulosclerosis are within the broader disorder of nephrotic syndrome, and are characterized by proteinuria. Minimal change disease can progress into focal segmental glomerulosclerosis, where the latter involve injury and scarring to the kidney in a focal, segmental pattern.

In some embodiments, the pharmaceutical compositions described herein are used for the treatment, prevention, and/or reduction of a risk of respiratory disease, disorder, or condition selected from chronic cough, atopic asthma, allergic rhinitis, sinusitis, hay fever, pneumonia, and pulmonary sepsis, or an organ disease, disorder, or condition selected from alcohol induced hepatitis, minimal change disease, and focal segmental glomerulosclerosis.

As noted above, in one aspect the present disclosure provides a method of treating, preventing, and/or reducing of a risk of respiratory disease, disorder, or condition selected from chronic cough, atopic asthma, allergic rhinitis, sinusitis, hay fever, pneumonia, and pulmonary sepsis, or an organ disease, disorder, or condition selected from alcohol induced hepatitis, minimal change disease, and focal segmental glomerulosclerosis, the method comprising administering an effective amount of a pharmaceutical composition described herein.

In some embodiments, the atopic (or allergic) asthma is triggered by an allergen such as an indoor, outdoor, or occupational allergen, including pollen, dust, an animal (e.g., cat dander or dog hair), or dust mites. In some embodiments, the atopic asthma patient also has another condition selected from seasonal allergies, eczema, and a food allergy.

In some embodiments, the disease, disorder, or condition is pulmonary sepsis. In some embodiments, the disease, disorder, or condition is sepsis or septic shock. In some embodiments, the disease, disorder, or condition is keratitis. In some embodiments, the disease, disorder, or condition is graft vs. host disease, such as graft vs. host disease after a corneal or organ transplant. In some embodiments, the disease, disorder, or condition is arthritis, osteoarthritis, or rheumatoid arthritis. In some embodiments, the disease, disorder, or condition is multiple sclerosis. In some embodiments, the disease, disorder, or condition is amyotrophic lateral sclerosis. In some embodiments, the disease, disorder, or condition is Alzheimer's disease. In some embodiments, the disease, disorder, or condition is Huntington's disease. In some embodiments, the disease, disorder, or condition is Parkinson's disease. In some embodiments, the disease, disorder, or condition is fibrosis.

In some embodiments, the disease, disorder, or condition is keratitis. In some embodiments, the disease, disorder, or condition is neurotrophic keratitis. In some embodiments, the disease, disorder, or condition is scleritis.

In some embodiments, the present disclosure provides use of the pharmaceutical composition described herein in the manufacture of a medicament for the treatment, prevention, and/or reduction of a risk of respiratory disease, disorder, or condition selected from chronic cough, atopic asthma, allergic rhinitis, sinusitis, hay fever, pneumonia, and pulmonary sepsis, or an organ disease, disorder, or condition selected from alcohol induced hepatitis, minimal change disease, and focal segmental glomerulosclerosis.

In some embodiments, the present disclosure provides a method of treating ethanol toxicity, comprising administering to a subject in need thereof an effective amount of a disclosed compound. In some embodiments, the present disclosure provides use of a disclosed compound or pharmaceutical composition described herein in the manufacture of a medicament for the treatment, prevention, and/or reduction of a risk of ethanol toxicity. In some embodiments, the present disclosure provides a method of treating hangover, comprising administering to a subject in need thereof an effective amount of a disclosed compound. In some embodiments, the present disclosure provides use of a disclosed compound or pharmaceutical composition described herein in the manufacture of a medicament for the treatment, prevention, and/or reduction of a risk of hangover.

In some embodiments, a method of the disclosure is directed to treatment of chronic cough. In some embodiments, a method of treating or reducing the risk of chronic cough comprises administering to a patient in need thereof an effective amount of a pharmaceutical composition disclosed herein. Generally, chronic cough is characterized as cough lasting greater than 8 weeks duration (see, e.g., Irwin et al., Chest, 2018; 153(1):196-209; Morice, A. H., European Respiratory J., 2004; 24:481-492). Chronic cough can be triggered by and/or arise from different underlying causes, such as asthma, gastroesophageal reflux disease (GERD), non-asthmatic eosinophilic bronchitis (NAEB), and upper airway cough syndrome, otherwise known as postnasal drip syndrome. A differential diagnosis of chronic cough excludes cough accompanied by fever, such as from a bacterial or viral infection; chronic obstructive pulmonary disease (COPD) and other non-asthmatic pulmonary diseases; cancer of the lung or esophagus; pneumonia; interstitial lung disease; and obstructive sleep apnea (see, e.g., Perotin et al., Ther Clin RiskManag, 2018: 14:1041-1051).

In some embodiments, the chronic cough for treatment is associated with upper airway cough syndrome.

In some embodiments, the chronic cough for treatment is associated with gastroesophageal reflux disease or laryngopharyngeal reflux disease.

In some embodiments, the chronic cough for treatment is associated with asthma.

In some embodiments, the chronic cough for treatment is associated with non-asthmatic eosinophilic bronchitis.

In some embodiments, the patient treated has a history of one or more of the following: treatment with angiotensin-converting enzyme (ACE) inhibitor, smoking, asthma, exposure to environmental respiratory irritants, and bronchitis.

In some embodiments, a method of the disclosure is directed to treatment of pneumonia. In some embodiments, the pneumonia is not associated or concurrent with acute respiratory distress syndrome (ARDS).

In some embodiments, the patient treated has pneumonia, wherein the pneumonia has a differential diagnosis from eosinophilic pneumonia (i.e., the pneumonia is not associated with eosinophilic pneumonia).

In some embodiments, the pneumonia treated is community-acquired pneumonia.

In some embodiments, the pneumonia treated is nocosomial pneumonia.

In some embodiments, the pneumonia treated is bacterial pneumonia or viral pneumonia.

In some embodiments, the patient treated is diagnosed with a bacterial infection by, among others, Streptococcus pneumoniae, Haemophilus influenzae, S. aureus, Group A streptococci, Moraxella catarrhalis, Klebsiella pneumoniae, Pseudomonas aeruginosa, Legionella spp, Mycoplasma pneumoniae, Chlamydia pneumoniae, or C. psittaci.

In some embodiments, the patient treated is diagnosed with a viral infection by influenza virus (e.g., influenza A or influenza B), respiratory syncytial virus (RSV), parainfluenza, metapneumovirus, coronavirus, rhinovirus, hantavirus, or adenovirus.

In some embodiments, the pneumonia treated is lobar pneumonia.

In some embodiments, the pneumonia treated is upper, middle or lower lobe pneumonia.

In some embodiments, the pneumonia treated is focal pneumonia, alveolar pneumonia, or interstitial pneumonia.

In some embodiments, the pneumonia treated is bronchial pneumonia.

In some embodiments, the method of the disclosure is directed to treatment of atopic asthma. In some embodiments, the method of the disclosure is directed to treatment of allergic rhinitis.

In some embodiments, a method of the disclosure is directed to treatment or reducing the risk of sepsis. In some embodiments, a method of the disclosure is directed to treatment of pulmonary sepsis or sepsis-induced lung injury. Generally, pulmonary sepsis or sepsis induced lung injury is characterized as lung injury arising from sepsis. The lung is the organ most often affected by sepsis primarily because pneumonia is often the starting point of the septic process, and disseminated infectious process is associated with a systemic inflammatory response (SIRS) in which the first organ to be affected is usually the lung.

In some embodiments, the pulmonary sepsis or sepsis induced lung injury treated is without (i.e., not associated with) acute respiratory distress syndrome (ARDS).

In some embodiments, a method of the disclosure is directed to treatment of alcohol poisoning. In some embodiments, a method of the disclosure is directed to treatment of alcohol-induced hepatitis. Generally, alcohol-induced hepatitis includes liver injury and associated inflammatory condition arising from chronic alcohol abuse. A prominent feature or marker for the disease is hyperbilirubinemia. In some embodiments, alcohol-induced hepatitis is distinguished from cirrhosis in that the former appears reversible while the latter is a permanent injury to the liver.

In some embodiments, the alcohol-induced hepatitis is without cirrhosis (i.e., not accompanied by cirrhosis).

In some embodiments, the patient treated for alcohol-induced hepatitis is determined to have elevated levels of aspartate aminotransferase (AST) and/or alanine aminotransferase (ALT) as compared to levels in a control group not afflicted with alcohol induced hepatitis.

In some embodiments, the levels of AST in the control group (i.e., without alcohol induced hepatitis) is about 8 to 48 IU/L and the levels of ALT in the control group is about 7 to 55 IU/L.

In some embodiments, the patient treated has an AST:ALT ratio of greater than 2:1. This ratio is characteristic in patients with alcoholic liver disease. Patients with a history of alcohol abuse but no significant alcoholic hepatitis or cirrhosis of the liver usually have an AST/ALT ratio less than 1.0.

In some embodiments, a method of the disclosure is directed to treatment of minimal change disease, sometimes referred to as lipoid nephrosis or nil disease. In some embodiments, a method of treating or reducing the risk of minimal change disease comprises administering to a patient in need thereof an effective amount of a compound disclosed herein. Generally, minimal change disease is a kidney disease arising from a histopathologic lesion in the glomerulus and is characterized by proteinuria leading to edema and intravascular volume depletion. Minimal change disease is a common form of nephrotic syndrome.

In some embodiments, the minimal change disease treated is associated with nephrotic syndrome.

In some embodiments, the minimal change disease treated is concurrent with proteinuria, particularly excessive proteinuria.

Minimal change disease can also advance to focal segmental glomerulosclerosis. Accordingly, in some embodiments, a method of the disclosure is directed to treatment of focal segmental glomerulosclerosis (FGS). In some embodiments, a method of treating or reducing the risk of FGS comprises administering to a patient in need thereof an effective amount of a compound disclosed herein. Generally, FGS describes both a common lesion in progressive kidney disease and excessive proteinuria and podocyte injury. The injury and scarring of the kidney is characterized by focal involvement in a segmental pattern. FGS is also a common cause of nephrotic syndrome.

In some embodiments, the FSGS treated is primary FSGS.

In some embodiments, the FSGS treated is secondary FSGS.

In some embodiments, the FSGS treated is familial FSGS. Autosomal dominant FSGS is associated with mutations in the gene encoding Inverted Formin 2 (INF2), alpha-actinin-4 gene ACTN4; the gene encoding TRPC6 cation channel protein; and the gene ARHGAP24 encoding the FilGAP protein (see, e.g., Pollak, M. R., Adv Chronic Kidney Dis., 2014, 21(5): 422-425). Recessive forms of FSGS are associated with mutations in the gene NPHS1 encoding nephrin; and the gene PLCE1 encoding phospholipase C epsilon 1 (see, e.g., Pollak, supra).

In some embodiments, the FSGS treated is associated with nephrotic syndrome.

In some embodiments, the FSGS treated is concurrent with kidney failure and/or proteinuria, particularly excessive proteinuria.

In some embodiments, the patient treated for FSGS has a prior history of minimal change disease.

In some embodiments, the pharmaceutical composition of the present disclosure comprising a compound or pharmaceutically acceptable salt thereof described herein is administered systemically to treat the indications described herein. In some embodiments, the pharmaceutical composition is administered orally.

In some embodiments, the pharmaceutical composition is a liquid. In some embodiments, the pharmaceutical composition is administered as a liquid via nasogastric tube.

A skilled person would understand that the disease, disorder, or condition listed herein may involve more than one pathological mechanism. For example, a disease, disorder, or condition listed herein may involve dysregulation in the immunological response and inflammatory response. Thus, the above categorization of a disease, disorder, or condition is not absolute, and the disease, disorder, or condition may be considered an immunological, an inflammatory, a cardiovascular, a neurological, and/or metabolic disease, disorder, or condition.

Individuals with deficiencies in aldehyde dehydrogenase are found to have high aldehyde levels and increased risk of Parkinson's disease (PNAS 110:636 (2013)) and Alzheimer's disease (BioChem Biophys Res Commun. 273:192 (2000)). In Parkinson's disease, aldehydes specifically interfere with dopamine physiology (Free Radic Biol Med, 51: 1302 (2011); Mol Aspects Med, 24: 293 (2003); Brain Res, 1145: 150 (2007)). In addition, aldehydes levels are elevated in multiple sclerosis, amyotrophic lateral sclerosis, autoimmune diseases such as lupus, rheumatoid arthritis, lupus, psoriasis, scleroderma, and fibrotic diseases, and increased levels of HNE and MDA are implicated in the progression of atherosclerosis and diabetes (J. Cell. Mol. Med., 15: 1339 (2011); Arthritis Rheum 62: 2064 (2010); Clin Exp Immunol, 101: 233 (1995); Int J Rheum Dis, 14: 325 (2011); JEADV 26: 833 (2012); Clin Rheumatol 25: 320 (2006); Gut 54: 987 (2005); J Am Soc Nephrol 20: 2119 (2009)). MDA is further implicated in the increased formation of foam cells leading to atherosclerosis (Leibundgut et al., Current Opinion in Pharmacology 13: 168 (2013)). Also, aldehyde-related toxicity plays an important role in the pathogenesis of many inflammatory lung diseases, such as asthma and chronic obstructive pulmonary disease (COPD) (Bartoli et al., Mediators of Inflammation 2011, Article 891752). Thus, compounds that reduce or eliminate aldehydes, such as compounds described herein, can be used to treat, prevent, and/or reduce a risk of an autoimmune, immune-mediated, inflammatory, cardiovascular, or neurological disease, disorder, or condition, or metabolic syndrome, or diabetes. For example, compounds described herein prevent aldehyde-mediated cell death in neurons. Further, compounds described herein downregulate a broad spectrum of pro-inflammatory cytokines and/or upregulate anti-inflammatory cytokines, which indicates that compounds described herein are useful in treating inflammatory diseases, such as multiple sclerosis and amyotrophic lateral sclerosis.

As discussed above, a disclosed compound may be administered to a subject in order to treat or prevent macular degeneration and other forms of retinal disease whose etiology involves the accumulation of A2E and/or lipofuscin. Other diseases, disorders, or conditions characterized by the accumulation of A2E may be similarly treated.

In one embodiment, a compound is administered to a subject that reduces the formation of A2E. For example, the compound may compete with PE for reaction with trans-RAL, thereby reducing the amount of A2E formed. In another embodiment, a compound is administered to a subject that prevents the accumulation of A2E. For example, the compound competes so successfully with PE for reaction with trans-RAL, no A2E is formed.

Individuals to be treated fall into three groups: (1) those who are clinically diagnosed with macular degeneration or other forms of retinal disease whose etiology involves the accumulation of A2E and/or lipofuscin on the basis of visual deficits (including but not limited to dark adaptation, contrast sensitivity and acuity) as determined by visual examination and/or electroretinography, and/or retinal health as indicated by fundoscopic examination of retinal and RPE tissue for drusen accumulations, tissue atrophy and/or lipofuscin fluorescence; (2) those who are pre-symptomatic for macular degenerative disease but thought to be at risk based on abnormal results in any or all of the same measures; and (3) those who are pre-symptomatic but thought to be at risk genetically based on family history of macular degenerative disease and/or genotyping results showing one or more alleles or polymorphisms associated with the disease. The compositions are administered topically or systemically at one or more times per month, week or day. Dosages may be selected to avoid side effects, if any, on visual performance in dark adaptation. Treatment is continued for a period of at least one, three, six, or twelve or more months. Patients may be tested at one, three, six, or twelve months or longer intervals to assess safety and efficacy. Efficacy is measured by examination of visual performance and retinal health as described above.

In one embodiment, a subject is diagnosed as having symptoms of macular degeneration, and then a disclosed compound is administered. In another embodiment, a subject may be identified as being at risk for developing macular degeneration (risk factors include a history of smoking, age, female sex, and family history), and then a disclosed compound is administered. In another embodiment, a subject may have dry AMD in both eyes, and then a disclosed compound is administered. In another embodiment, a subject may have wet AMD in one eye but dry AMD in the other eye, and then a disclosed compound is administered. In yet another embodiment, a subject may be diagnosed as having Stargardt disease and then a disclosed compound is administered. In another embodiment, a subject is diagnosed as having symptoms of other forms of retinal disease whose etiology involves the accumulation of A2E and/or lipofuscin, and then the compound is administered. In another embodiment a subject may be identified as being at risk for developing other forms of retinal disease whose etiology involves the accumulation of A2E and/or lipofuscin, and then the disclosed compound is administered. In some embodiments, a compound is administered prophylactically. In some embodiments, a subject has been diagnosed as having the disease before retinal damage is apparent. For example, a subject is found to carry a gene mutation for ABCA4 and is diagnosed as being at risk for Stargardt disease before any ophthalmologic signs are manifest, or a subject is found to have early macular changes indicative of macular degeneration before the subject is aware of any effect on vision. In some embodiments, a human subject may know that he or she is in need of the macular generation treatment or prevention.

In some embodiments, a subject may be monitored for the extent of macular degeneration. A subject may be monitored in a variety of ways, such as by eye examination, dilated eye examination, fundoscopic examination, visual acuity test, and/or biopsy. Monitoring can be performed at a variety of times. For example, a subject may be monitored after a compound is administered. The monitoring can occur, for example, one day, one week, two weeks, one month, two months, six months, one year, two years, five years, or any other time period after the first administration of a compound. A subject can be repeatedly monitored. In some embodiments, the dose of a compound may be altered in response to monitoring.

In some embodiments, the disclosed methods may be combined with other methods for treating or preventing macular degeneration or other forms of retinal disease whose etiology involves the accumulation of A2E and/or lipofuscin, such as photodynamic therapy. For example, a patient may be treated with more than one therapy for one or more diseases or disorders. For example, a patient may have one eye afflicted with dry form AMD, which is treated with a compound of the invention, and the other eye afflicted with wet form AMD which is treated with, e.g., photodynamic therapy.

In some embodiments, a compound for treating or preventing macular degeneration or other forms of retinal disease whose etiology involves the accumulation of A2E and/or lipofuscin may be administered chronically. The compound may be administered daily, more than once daily, twice a week, three times a week, weekly, biweekly, monthly, bimonthly, semiannually, annually, and/or biannually.

Sphingosine-1 phosphate, a bioactive signaling molecule with diverse cellular functions, is irreversibly degraded by the endoplasmic reticulum enzyme sphingosine-1 phosphate lyase, generating trans-2-hexadecenal and phosphoethanolamine. It has been demonstrated that trans-2-hexadecenal causes cytoskeletal reorganization, detachment, and apoptosis in multiple cell types via a JNK-dependent pathway. See Biochem Biophys Res Commun. 2012 Jul. 20; 424(1):18-21. These findings and the known chemistry of related α,β-unsaturated aldehydes raise the possibility that trans-2-hexadecenal interact with additional cellular components. It was shown that it reacts readily with deoxyguanosine and DNA to produce the diastereomeric cyclic 1,N(2)-deoxyguanosine adducts 3-(2-deoxy-β-d-erythro-pentofuranosyl)-5,6,7,8-tetrahydro-8R-hydroxy-6R-tridecylpyrimido[1,2-a]purine-10(3H)one and 3-(2-deoxy-β-d-erythro-pentofuranosyl)-5,6,7,8-tetrahydro-8S-hydroxy-6S-tridecylpyrimido[1,2-a]purine-10(3H)one. These findings demonstrate that trans-2-hexadecenal produced endogenously by sphingosine-1 phosphate lyase react directly with DNA forming aldehyde-derived DNA adducts with potentially mutagenic consequences.

Succinic semialdehyde dehydrogenase deficiency (SSADHD), also known as 4-hydroxybutyric aciduria or gamma-hydroxybutyric aciduria, is the most prevalent autosomal-recessively inherited disorder of GABA metabolism (Vogel et al. 2013). It manifests a phenotype of developmental delay and hypotonia in early childhood, and severe expressive language impairment and obsessive-compulsive disorder in adolescence and adulthood. Epilepsy occurs in half of patients, usually as generalized tonic-clonic seizures although sometimes absence and myoclonic seizures occur (Pearl et al. 2014). Greater than two-thirds of patients manifest neuropsychiatric problems (i.e., ADHD, OCD and aggression) in adolescence and adulthood, which can be disabling. Metabolically, there is accumulation of the major inhibitory neurotransmitter GABA and gamma-hydroxybutyrate (GHB), a neuromodulatory monocarboxylic acid (Snead and Gibson 2005). In addition, several other intermediates specific to this disorder have been detected both in patients and the corresponding murine model. Vigabatrin (VGB; 7-vinylGABA), an irreversible inhibitor of GABA-transaminase, is a logical choice for treatment of SSADH deficiency because it will black the conversion of GABA to GHB. Outcomes have been mixed, and in selected patients treatment has led to deterioration (Good 2011; Pellock 2011; Escalera et al. 2010; Casarano et al. 2011; Matern et al. 1996; Al-Essa et al. 2000). Targeted therapy for SSADHD remains elusive and, to date, interventions are only palliative. Accordingly, in some embodiments, the present invention provides a method of treating SSADHD, comprising administering to a subject in need thereof an effective amount of a disclosed compound or a pharmaceutically acceptable salt thereof. In some embodiments, the method ameliorates a symptom of SSADHD selected from developmental delay, hypotonia, severe expressive language impairment, obsessive-compulsive disorder, epilepsy (e.g., generalized tonic-clonic seizures, myoclonic seizures) or a neuropsychiatric disorder (e.g., ADHD, OCD and aggression). In some embodiments, the method reduces bioaccumulation of GHB and/or GABA.

### 5. Pharmaceutically Acceptable Compositions

The compounds and compositions, according to the method of the present invention, are administered using any amount and any route of administration effective for treating or lessening the severity of a disease, disorder, or condition provided above. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “unit dosage form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, the compounds of the invention are administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compounds of the invention can also be administered topically, such as directly to the eye, e.g., as an eye-drop or ophthalmic ointment. Eye drops typically comprise an effective amount of at least one compound of the invention and a carrier capable of being safely applied to an eye. For example, the eye drops are in the form of an isotonic solution, and the pH of the solution is adjusted so that there is no irritation of the eye. In many instances, the epithelial barrier interferes with penetration of molecules into the eye. Thus, most currently used ophthalmic drugs are supplemented with some form of penetration enhancer. These penetration enhancers work by loosening the tight junctions of the most superior epithelial cells (Burstein, 1985, Trans Ophthalmol Soc U K 104(Pt 4): 402-9; Ashton et al., 1991, J Pharmacol Exp Ther 259(2): 719-24; Green et al., 1971, Am J Ophthalmol 72(5): 897-905). The most commonly used penetration enhancer is benzalkonium chloride (Tang et al., 1994, J Pharm Sci 83(1): 85-90; Burstein et al., 1980, Invest Ophthalmol Vis Sci 19(3): 308-13), which also works as preservative against microbial contamination. It is typically added to a final concentration of 0.01-0.05%.

Topical administration may be in the form of a cream, suspension, emulsion, ointment, drops, oil, lotion, patch, tape, inhalant, spray, or controlled release topical formulations including gels, films, patches, and adhesives. Intra-ocular administration may take the form of subconjunctival, subtenon's capsule, retrobulbar or intravitreal injections, depots or implants. Compounds administered by these routes may be in solution or suspension form. Administration of compounds by depot injection may contain pharmaceutically acceptable carriers or excipients; these may be natural or synthetic and may be biodegradable or non-biodegradable and facilitate drug release in a controlled manner. Implants used for controlled release of compound may be composed of natural or synthetic, biodegradable or non-biodegradable materials. The carrier is acceptable in that it is compatible with the other components of the composition and is not injurious to the patient. Some examples of carriers include (1) sugars such as lactose glucose and sucrose, (2) starches such as corn starch and potato starch, (3) cellulose and (4) cyclodextrins. A useful topical formulation is described in PCT publication WO 2011/072141, the contents of which are herein incorporated by reference.

Formulations for topical administration to the skin can include, for example, ointments, creams, gels and pastes comprising the primary amine compound in a pharmaceutical acceptable carrier. The formulation of the primary amine compound for topical use includes the preparation of oleaginous or water-soluble ointment bases, as is well known to those in the art. For example, these formulations may include vegetable oils, animal fats, and, for example, semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin and glyceryl monostearate. Various water-soluble ointment bases may also be used, including glycol ethers and derivatives, polyethylene glycols, polyoxyl 40 stearate and polysorbates.

The formulations for topical administration may contain the compound used in the present application at a concentration in the range of 0.001-10%, 0.05-10%, 0.1-10%, 0.2-10%, 0.5-10%, 1-10%, 2-10%, 3-10%, 4-10%, 5-10%, or 7-10% (weight/volume), or in the range of 0.001-2.0%, 0.001-1.5%, or 0.001-1.0%, (weight/volume), or in the range of 0.05-2.0%, 0.05-1.5%, or 0.05-1.0%, (weight/volume), or in the range of 0.1-5.0%, 0.1-2.0%, 0.1-1.5%, or 0.1-1.0% (weight/volume), or in the range of 0.5-5.0%, 0.5-2.0%, 0.5-1.5%, or 0.5-1.0% (weight/volume), or in the range of 1-5.0%, 1-2.0%, or 1-1.5% (weight/volume). The formulations for topical administration may also contain the compound used in the present application at a concentration in the range of 0.001-2.5%, 0.01-2.5%, 0.05-2.0%, 0.1-2.0%, 0.2-2.0%, 0.5-2.0%, or 1-2.0% (weight/weight), or in the range of 0.001-2.0%, 0.001-1.5%, 0.001-1.0%, or 0.001-5% (weight/weight).

In an eye drop formulation the composition may contain the active compound at a concentration of 0.01-20%, 0.02-15%, 0.04-10%, 0.06-5%, 0.08-1%, or 0.09-0.5% (weight/volume) with or without pH and/or osmotic adjustment to the solution. More particularly, the eye drop formulation may contain a compound described herein at a concentration of 0.09-0.5% (weight/volume), such as 0.1%, 0.25%, or 0.5%.

In one exemplification, the pharmaceutical compositions encompass a composition made by admixing a therapeutically effective amount of a compound described herein with an oligomeric or a polymeric carrier such as a cyclodextrin, or chemically modified cyclodextrin, including trimethyl-β-cyclodextrin, 2-hydroxyethyl-β-cyclodextrin, 2-hydroxypropyl-β-cyclodextrin, 3-hydroxypropyl-β-cyclodextrin, and β-cyclodextrin sulfobutylether sodium salt (or potassium salt). Exemplifying an oligomeric or a polymeric carrier is β-cyclodextrin sulfobutylether sodium salt. The amount of β-cyclodextrin sulfobutylether sodium salt in the composition may range from about 0.01% to 30% weight/volume. In one illustration, the concentration of β-cyclodextrin sulfobutylether sodium salt is 5-25% weight/volume. Further illustrating the concentration of β-cyclodextrin sulfobutylether sodium salt is 6-20% weight/volume. In one exemplification the concentration of β-cyclodextrin sulfobutylether is 6-12% weight/volume. Further exemplifying the concentration of β-cyclodextrin sulfobutylether is 9-10% weight/volume, including 9.5% weight/volume. The amount of the compound described herein in the composition may range 0.01-20%, 0.02-15%, 0.04-10%, 0.06-5%, 0.08-1%, or 0.09-0.5% (weight/volume). More particularly, the composition may contain a compound described herein at a concentration of 0.09-0.5% (weight/volume), such as 0.1%.

The compounds described herein may be administered orally and as such the pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

For oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, croscarmellose or its sodium salt, and the like. Diluents, include, e.g., lactose, dextrose, sucrose, mannitol, sorbitol, cellulose and/or glycine.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

For administration by inhalation, the compounds are delivered in the form of an aerosol spray from pressured container or dispenser, which contains a suitable propellant, e.g., a gas such as carbon dioxide, or a nebulizer.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Parenteral formulations comprising a compound described herein can be prepared in aqueous isotonic solutions or suspensions, and suppositories are advantageously prepared from fatty emulsions or suspensions. The formulations may be sterilized and/or contain adjuvants, such as preserving, stabilizing, wetting or emulsifying agents, solution promoters, salts for regulating the osmotic pressure and/or buffers. In addition, they may also contain other therapeutic agents. The compositions are prepared according to conventional methods, and may contain about 0.1 to 75%, preferably about 1 to 50%, of a compound described herein.

In certain embodiments, the present invention is directed to a composition, as described herein, comprising a prodrug of a compound of formula I or VI, or a pharmaceutically acceptable salt thereof. The term “prodrug,” as used herein, means a compound that is convertible in vivo by metabolic means (e.g. by hydrolysis) to a compound of formula I or VI, or a pharmaceutically acceptable salt thereof. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). Design and Application of Prodrugs, Textbook of Drug Design and Development, Chapter 5, 113-191 (1991), Bundgaard, et al., Journal of Drug Delivery Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is hereby incorporated by reference in its entirety.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compounds are prepared according to the following general procedures. It will be appreciated that, although the general methods depict the synthesis of particular compounds of the present invention, the following general methods, and other methods known to one of ordinary skill in the art, can be applied to all compounds and subclasses and species of each of these compounds, as described herein.

### Abbreviations

equiv or eq: molar equivalents
o/n: overnight
rt: room temperature
UV: ultra violet
HPLC: high pressure liquid chromatography
Rt: retention time
LCMS or LC-MS: liquid chromatography-mass spectrometry
NMR: nuclear magnetic resonance
CC: column chromatography
TLC: thin layer chromatography
sat: saturated
aq: aqueous
Ac: acetyl
ACN or MeCN: acetonitrile
DCM: dichloromethane
DCE: dichloroethane
DEA: diethylamine
DMF: dimethylformamide
DMSO: dimethylsulfoxide
DIPEA: diisopropylethylamine
EA or EtOAc: ethyl acetate
BINAP: (±)-2,2′-Bis(diphenylphosphino)-1,1′-binaphthalene
TEA: triethylamine
THF: tetrahydrofuran
TBS: tert-butyldimethylsilyl
KHMDS: potassium hexamethyl disilylazide
Tf: trifluoromethanesulfonate
Ms: methanesulfonyl
NBS: N-bromosuccinimide
NMP: N-methyl pyrrolidinone
PE: petroleum ether
TFA: trifluoroacetic acid
FA: formic acid
MMPP: magnesium monoperoxyphthalate
HATU: 1-[Bis(dimethylamino)methylene]-1H-1,2,3-triazolo[4,5-b]pyridinium 3-oxid Hexafluorophosphate
Cy: cyclohexyl
Tol: toluene
PTSA: p-Toluenesulfonic acid
NMP: N-Methyl-2-pyrrolidone
TFA: 1,1,1,-trifluoroacetone

### Example 1: Synthesis of Thiophene Core Compounds

### Synthesis of 2-(3-aminobenzo[b]thiophen-2-yl)-1, 1, 1-trifluoropropan-2-ol (I-1)

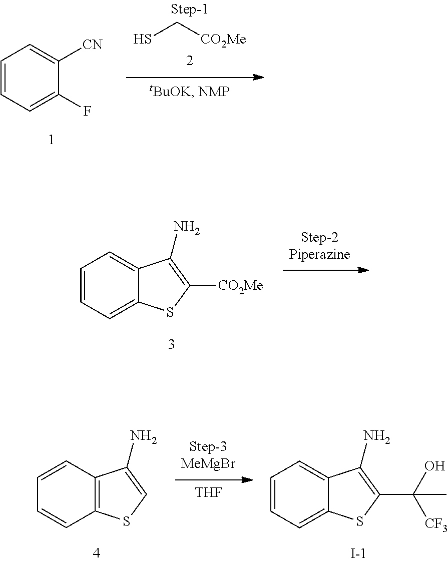

### Synthesis of methyl 3-aminobenzo[b]thiophene-2-carboxylate 3

To a solution of 2-fluorobenzonitrile 1 (30.0 g, 1.0 eq) in DMF (165 mL) was added methyl thioglycolate 2 (2.5 eq) followed by t-BuOK (2.5 eq) at 0-5° C. After 15 min, the reaction mixture was slowly warmed to room temperature and stirred for 4 h. After consumption of the starting material (determined by TLC), the reaction mixture was poured into crushed ice and the precipitated solid was filtered. The collected solid was dried to obtain 3 (37.0 g, 72%) as an off white solid. ¹H NMR (CD₃OD, 500 MHz): δ 7.91 (d, 1H), 7.71 (d, 1H), 7.46 (t, 1H), 7.35 (t, 1H), 3.84 (s, 3H).

### Synthesis of benzo[b]thiophen-3-amine 4

To a solution of 3 (20.0 g, 1.0 eq) in NMP (100 mL) was added piperazine (5.0 eq) and the reaction mixture was stirred for 6 h at 180° C. After consumption of the starting material (determined by TLC), the reaction mixture was diluted with ice cold water and extracted with ethyl acetate. The organic layer was separated, dried over Na₂SO₄ and evaporated under reduced pressure to obtain 4 (13.0 g, 90%) as a pale brown solid. ¹H NMR (DMSO-d₆, 500 MHz): δ 7.83-7.79 (m, 2H), 7.34-7.28 (m, 2H), 6.16 (s, 1H), 5.27 (s, 2H).

### Synthesis of I-1

To a solution of 4 (5.0 g, 1 eq) in THE (40 mL) was added methyl magnesium bromide (3.0 eq, 2M solution) at 0-5° C. under nitrogen. After stirring for 30 min, trifluoroacetone (1.2 eq) was added at 0-5° C. and the reaction temperature was slowly raised to room temperature. After consumption of the starting material (determined by TLC), the reaction mixture was quenched with NH₄Cl solution and extracted with ethyl acetate. The organic layer was separated, dried over Na₂SO₄ and evaporated under reduced pressure to obtain I-1 (1.59 g, 18%) as a yellow solid. ¹H NMR (DMSO-d₆; 500 MHz): δ 7.85-7.83 (m, 1H), 7.77-7.75 (m, 1H), 7.36-7.31 (m, 2H), 7.13 (s, 1H), 5.41 (s, 2H), 1.74 (s, 3H); Mass: m/z 262.23 (+ve); HPLC purity: 98.85%.

### Synthesis of 2-(3-amino-4-bromobenzo[b]thiophen-2-yl)-1, 1, 1-trifluoropropan-2-ol (I-2)

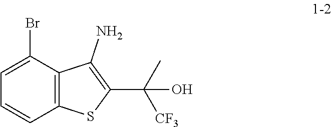 [SMILES: CC(O)(c1sc2cccc(Br)c2c1N)C(F)(F)F]

### Synthesis of I-2

Using procedures described for the synthesis of I-1, compound I-2 was prepared from 2-fluoro-6-bromobenzonitrile. 1-2 was obtained as a pale yellow solid (3.0 g, 41%). 1H NMR (DMSO-d6, 500 MHz): δ 7.86 (d, 1H), 7.57 (d, 1H), 7.36 (s, 1H), 7.23 (t, 1H), 5.51 (s, 2H), 1.75 (s, 3H); Mass: m/z 340.07 (−ve); HPLC purity: 99.58%.

### Synthesis of 2-(3-amino-6-(trifluoromethyl)benzo[b]thiophen-2-yl)-1,1,1-trifluoropropan-2-ol (I-3)

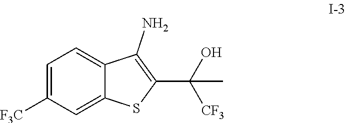 [SMILES: CC(O)(c1sc2cc(C(F)(F)F)ccc2c1N)C(F)(F)F]

### Synthesis of I-3

Using procedures described for the synthesis of I-1, compound I-3 was prepared from 2-fluoro-4-(trifluoromethyl)benzonitrile. 1-3 was obtained as a pale yellow solid (1.9 g, 42%). ¹H NMR (DMSO-d₆, 400 MHz): δ 8.28 (s, 1H), 8.07 (d, 1H), 7.65 (dd, 1H), 7.26 (s, 1H), 5.57 (s, 2H), 1.77 (s, 3H); Mass: m/z 330.27 (+ve); HPLC purity: 98.06%.

### Synthesis of 2-(3-Aminobenzo[b]thiophen-4-yl)-1,1,1-trifluoropropan-2-ol (I-4)

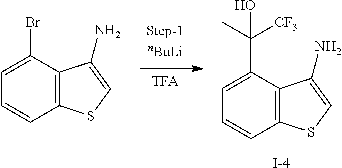

### Synthesis of I-4

To a solution of 3-Amino-4-bromo[b]benzothiophene (1 g, 1 eq, obtained using procedures described for the synthesis of benzo[b]thiophen-3-amine in the synthesis for I-1, using 2-fluoro-6-bromobenzonitrile in place of 2-fluorobenzonitrile) in THE was added n-butyl lithium (3.0 eq, 1.6 M solution in hexane) at −70° C. under nitrogen. After stirring for 30 min, trifluoroacetone (1.2 eq) was added at −70° C. and the reaction temperature was slowly raised to room temperature. After consumption of the starting material (determined by TLC), the reaction mixture was quenched with NH₄Cl solution and extracted with ethyl acetate. The organic layer was separated, dried over Na₂SO₄ and evaporated under reduced pressure to obtain crude I-4 as a pale yellow solid. The crude material was purified by F/C chromatography to provide a pure sample of I-4 as an off-white solid (127 mg, 11%). ¹H NMR (DMSO-d₆, 400 MHz): δ 7.84 (d, 1H), 7.54 (dd˜t, 1H), 7.32 (d, 1H), 6.34 (s, 1H), 5.58 (s, 2H), 4.90 (S, 1H), 1.51 (s, 3H); Mass: m/z 262 (+ve); HPLC purity: 98.06%.

### Synthesis of 2-(3-aminobenzo[b]thiophen-4-yl) propan-2-ol (I-5)

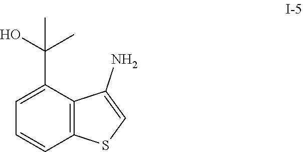

### Synthesis of I-5

Using procedures described for the synthesis of I-4, compound I-5 was prepared using acetone in place of TFA. I-5 was purified by silica gel column (1:5 ethyl acetate: hexane) and obtained as a pale yellow solid (5.0 mg, 2.7). ¹H NMR (DMSO-d₆; 500 MHz): δ 7.87 (d, 1H), 7.52 (d, 1H), 7.18 (t, 1H), 6.45 (s, 1H), 5.24 (bs, 2H); Mass: m/z 206.06 (−ve).

### Synthesis of 2-(3-Amino-4-(trifluoromethyl) benzo[b]thiophen-2-yl)-1,1,1-trifluoropropan-2-ol (I-14)

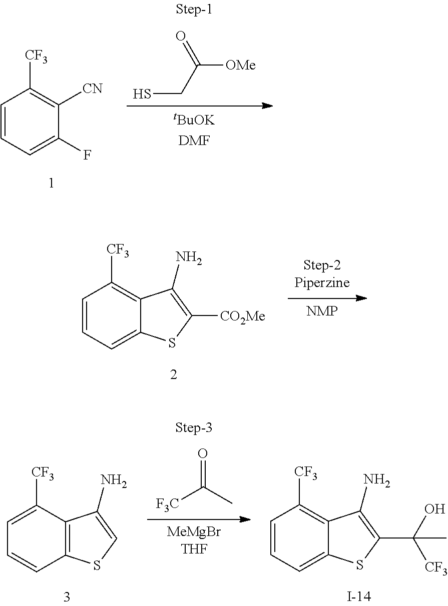

### Synthesis of Methyl 3-amino-4-(trifluoromethyl) benzo[b]thiophene-2-carboxylate 2

To a stirred solution of 2-fluoro-6-(trifluoromethyl) benzonitrile 1 (2.0 g, 1.0 eq) in DMF (10 mL) was added methyl thioglycolate (2.5 eq) followed by t-BuOK (1.5 eq) at 0-5° C. After 15 min, the reaction mixture was slowly warmed to room temperature, and stirred for 6 h. After consumption of starting material (by TLC), the reaction mass was poured into crushed ice, and stirred for 1 h at room temperature, obtained solid was filtered and dried to obtain 2 (0.75 g, 25%) as a reddish-brown flake. ¹H NMR (DMSO-d₆, 400 MHz): δ 7.90-7.85 (m, 2H), 7.82-7.79 (m, 1H), 4.26 (s, 2H), 3.66 (s, 3H).

### Synthesis of 4-(Trifluoromethyl) benzo[b]thiophen-3-amine 3

To a stirred solution of 2 (0.7 g, 1.0 eq) in NMP (3.5 mL) was added piperazine (0.66 g, 3 eq) and stirred the reaction mass for 6 h at 180° C. After consumption of starting material (by TLC), the reaction mass was cooled room temperature, diluted with ice cold water and extracted with ethyl acetate. The organic layer was separated, dried over Na₂SO₄ and evaporated under reduced pressure to obtain 3 (0.3 g, 54%) as a pale brown solid. ¹H NMR (DMSO-d₆, 500 MHz): δ 8.20 (d, 1H), 7.76 (d, 1H), 7.45 (t, 1H), 6.78 (s, 1H), 4.75 (bs, 2H).

### Synthesis of I-14

To a stirred solution of 3 (0.3 g, 1.0 eq) in THE (10 mL) was added methyl magnesium bromide (3.5 eq, 2M solution) at 0° C. under nitrogen. After stirring for 1 h, trifluoro acetone (1.5 eq) was added and stirred the reaction mixture for 4 h at room temperature. After consumption of starting material (by TLC), the reaction mixture was quenched with saturated NH₄Cl solution at 0° C. and extracted with ethyl acetate. The organic layer was separated, dried over Na₂SO₄ and evaporated under reduced pressure to give crude compound which was purified by column chromatography using 60-120 silica gel eluted with ethyl acetate:hexane (15:85); pure fractions were distilled off to obtain I-14 (155 mg, 34%) as an off white solid. ¹H NMR (DMSO-d₆, 400 MHz): δ 8.20-8.19 (d, 1H), 7.81-7.80 (d, 1H), 7.53-7.49 (m, 2H) 5.04 (bs, 2H), 1.79 (s, 3H).

### Synthesis of 2-(3-Amino-7-(trifluoromethyl) benzo[b]thiophen-2-yl)-1,1,1-trifluoropropan-2-ol (I-15)

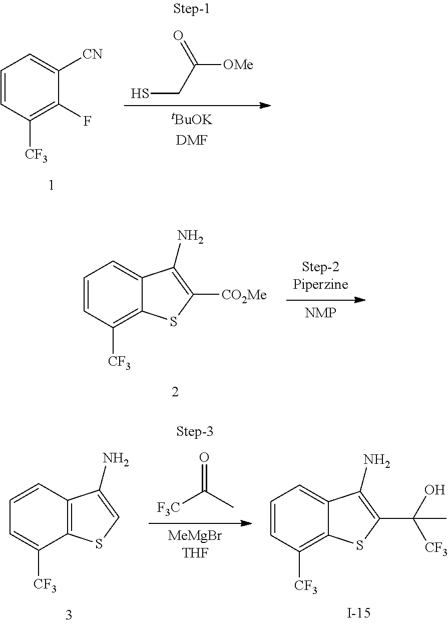

### Synthesis of Methyl 3-amino-7-(trifluoromethyl) benzo[b]thiophene-2-carboxylate 2

To a stirred solution of 2-fluoro-3-(trifluoromethyl) benzonitrile 1 (2.0 g, 1.0 eq) in DMF (10 mL) was added methyl thioglycolate (2.5 eq) followed by t-BuOK (1.5 eq) at 0-5° C. After 15 min, the reaction mixture was slowly warmed to room temperature and stirred for 6 h. After consumption of starting material (by TLC), the reaction mass was poured into crushed ice, and stir for 1 h at room temperature, the precipitated solid was filtered and dried to obtain 2 (2.6 g, 89%) as an off white solid. ¹H NMR (DMSO-d₆, 400 MHz): δ 8.66 (s, 1H), 8.10 (d, 1H), 7.80 (dd, 1H), 3.81 (s, 3H).

### Synthesis of 7-(Trifluoromethyl) benzo[b]thiophen-3-amine 3

To a stirred solution of 2 (2.6 g, 1.0 eq) in NMP (13 mL) was added piperazine (2.5 g, 3 eq) and stirred the reaction mass for 6 h at 180° C. After consumption of starting material (by TLC), the reaction mass cooled room temperature, diluted with ice cold water and then extracted ethyl acetate. The organic layer was separated, dried over Na₂SO₄ and evaporated under reduced pressure to obtain 3 (1.7 g, 82%) as a pale brown solid. ¹H NMR (DMSO-d₆, 400 MHz): δ 8.46 (d, 1H), 7.94 (d, 1H), 7.63 (t, 1H), 7.31 (s, 1H), 5.54 (bs, 2H).

### Synthesis of I-15

To a stirred solution of 3 (1.7 g, 1.0 eq) in THE (20 mL) was added methyl magnesium bromide (3.0 eq, 2M solution) at 0° C. under nitrogen. After stirring for 1 h, trifluoro acetone (1.25 eq) was added and stirred the reaction mixture for 4 h at room temperature. After consumption of starting material (by TLC), the reaction mixture was quenched with NH₄Cl solution at 0° C. and extracted with ethyl acetate. The organic layer was separated, dried over Na₂SO₄ and evaporated under reduced pressure to give crude compound which was purified by column chromatography silica gel eluted with ethyl acetate: hexane(15:85), pure fractions were distilled off to obtain I-15 (250 mg, 10%) as an off white solid. ¹H NMR (DMSO-d₆, 400 MHz): δ 8.17 (d, 1H), 7.76 (d, 1H), 7.56 (t, 1H), 7.26 (bs, 1H), 5.58 (bs, 2H), 1.78 (s, 3H).

### Synthesis of 2-(3-Amino-5-(trifluoromethyl) benzo[b]thiophen-2-yl)-1,1,1-trifluoropropan-2-ol (I-16)

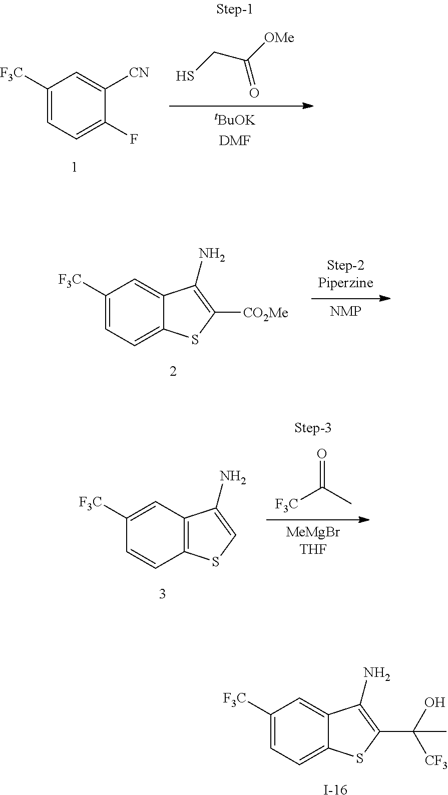

### Synthesis of Methyl 3-amino-5-(trifluoromethyl) benzo[b]thiophene-2-carboxylate 2

To a stirred solution of 2-fluoro-5-(trifluoromethyl) benzonitrile 1 (2.0 g, 1.0 eq) in DMF (10 mL) was added methyl thioglycolate (2.5 eq) followed by t-BuOK (1.5 eq) at 0-5° C. After 15 min, the reaction mixture was slowly warmed to room temperature and stirred for 5 h. After consumption of starting material (by TLC), the reaction mass was poured into crushed ice, and stir for 1 h at room temperature, the precipitated solid was filtered and dried to obtain 2 (2.3 g, 79%) as an off white solid. ¹H NMR (DMSO-d₆, 400 MHz): δ 8.66 (s, 1H), 8.19 (d, 1H), 7.80 (dd, 1H) 3.81 (s, 3H).

### Synthesis of 5-(Trifluoromethyl) benzo[b]thiophen-3-amine 3

To a stirred solution of 2 (2.3 g, 1.0 eq) in NMP (11.5 mL) was added piperazine (2.15 g, 3 eq) and stirred the reaction mass for 5 h at 200° C. After consumption of starting material (by TLC), the reaction mass was cooled room temperature and diluted with ethyl acetate. The organic layer was washed with ice cold water, separated, dried over Na₂SO₄ and evaporated under reduced pressure to obtain 3 (1.2 g, 66%) as an off white solid (Low melting solid). ¹H NMR (DMSO-d₆, 500 MHz): δ 8.30 (t, 1H), 8.05 (d, 1H), 7.60-7.57 (m, 1H), 6.35 (s, 1H), 5.54 (bs, 2H).

### Synthesis I-16

To a stirred solution of 3 (0.5 g, 1.0 eq) in THE (10 mL) was added methyl magnesium bromide (3.0 eq, 2M solution) at 0° C. under nitrogen. After stirring for 1 h, trifluoro acetone (1.5 eq) was added and stirred the reaction mixture for 3 h at room temperature. After consumption of starting material (by TLC), the reaction mixture was quenched with NH₄Cl solution at 0° C. and extracted with ethyl acetate. The organic layer was separated, dried over Na₂SO₄ and evaporated under reduced pressure to give crude compound which was purified by washings with n-hexane to obtain I-16 (400 mg, 52.7%) as an off white solid. ¹H NMR (DMSO-d₆, 400 MHz): δ 8.33 (s, 1H), 8.03 (d, 1H), 7.63-7.61 (m, 1H), 5.63 (bs, 2H), 1.77 (s, 3H).

### Example 2: Synthesis of Indazole Core Compounds

### Synthesis of 2-(3-amino-1H-indazol-4-yl) propan-2-ol (I-6)

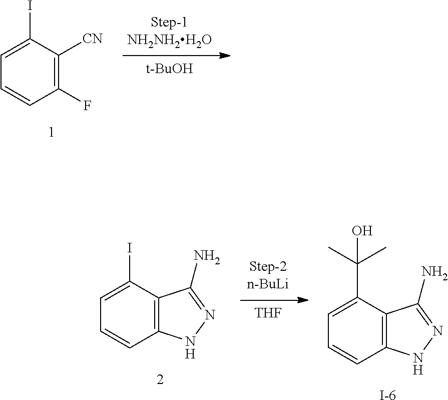

### Synthesis of 4-iodo-1H-indazol-3-amine 2

To a solution of 2-fluoro-6-iodobenzonitrile 1 (3.0 g, 1.0 eq) in t-butanol (60 mL) was added hydrazine hydrate (2.0 vol) at room temperature. The reaction mixture was stirred for 5 h at 105-110° C. After consumption of the starting material (determined by TLC), the reaction mixture was allowed to cool to room temperature, poured into ice cold water and extracted with ethyl acetate. The organic layer was separated, dried over Na₂SO₄ and evaporated under reduced pressure to obtain 2 (3.0 g, 96%) as an off white solid. ¹H NMR (DMSO-d₆, 400 MHz): δ 11.77 (s, 1H), 7.34 (d, 1H), 7.28 (d, 1H), 6.95-6.91 (m, 1H), 5.03 (s, 2H).

### Synthesis of I-6

To a solution of 2 (1.0 g, 1.0 eq) in THF was added n-butyl lithium (5.0 eq, 1.6M solution) at −70° C. under nitrogen atmosphere. After stirring for 30 min, acetone (5.0 eq) was added at −70° C. and the reaction temperature was slowly raised to room temperature. The reaction mixture was stirred for 2 h at room temperature. After consumption of the starting material (determined by TLC), the reaction mixture was quenched with NH₄Cl solution and extracted with ethyl acetate. The organic layer was separated, dried over Na₂SO₄ and evaporated under reduced pressure to give crude product which was purified by silica gel column chromatography to obtain I-6 (15.0 mg, 1.5%) as a tan solid. ¹H NMR (DMSO-d₆, 400 MHz): δ 11.48 (s, 1H), 7.15-7.07 (m, 2H), 6.78 (d, 1H), 5.83 (s, 1H), 5.68 (s, 2H), 1.59 (s, 6H); Mass: m/z 192.01 (+ve); HPLC purity: 95.69%.

### Synthesis of 2-(3-amino-1H-indazol-4-yl)-1, 1, 1-trifluoropropan-2-ol (I-9)

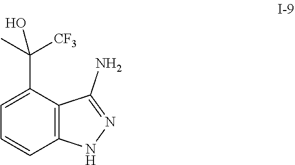 [SMILES: CC(O)(c1cccc2[nH]nc(N)c12)C(F)(F)F]

### Synthesis of I-9

Using procedures described for the synthesis of I-6, compound I-9 was prepared starting from 2-fluoro-6-bromobenzonitrile in place of 2-fluoro-6-iodobenzonitrile, and using TFA in place of acetone in step 2. I-9 was obtained as light brown solid (120 mg, 8%). ¹H NMR (DMSO-d₆, 400 MHz): δ 11.81 (s, 1H), 7.79 (s, 1H), 7.31-7.29 (m, 1H), 7.19 (t, 1H), 6.94 (d, 1H), 5.49 (s, 2H), 1.79 (s, 3H); Mass: m/z 246.30 (+ve); HPLC purity: 95.15%.

### Example 3: Synthesis of Dihydroquinoxalinone Core Compounds

### Synthesis of (R)-3-(Hydroxymethyl)-7-methoxy-3, 4-dihydroquinoxalin-2(1H)-one (I-7)

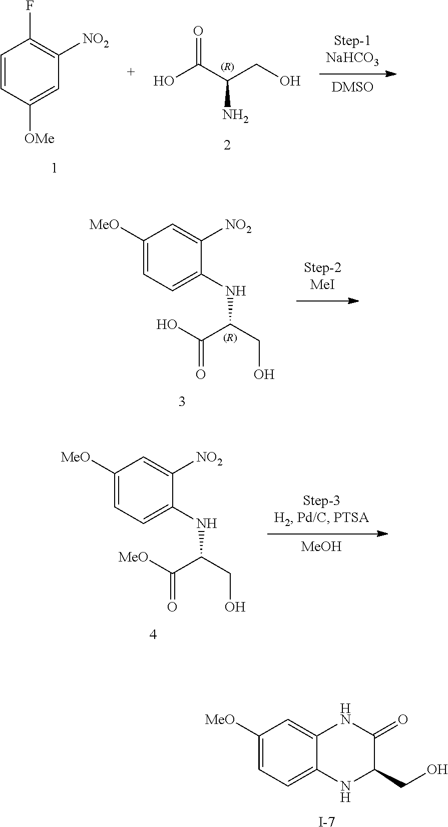

### Synthesis of (R)-Methyl 3-hydroxy-2-((4-methoxy-2-nitrophenyl) amino) propanoate 4

To a solution of 1-fluoro-4-methoxy-2-nitrobenzene 1 (5.0 g, 1.0 eq) in DMSO (25.0 mL) was added NaHCO₃ (6.0 eq) followed by D-serine 2 (2.0 eq) at room temperature. The reaction mixture was stirred for 4 h at 100-105° C. After consumption of the starting material (determined by TLC), the reaction mixture was cooled to room temperature and then methyl iodide (2.0 eq) was added and the reaction mixture was stirred for 4 h at room temperature. After consumption of the starting material (determined by TLC), the reaction mixture was poured into ice cold water and extracted with ethyl acetate. The organic layer was separated, dried over Na₂SO₄ and evaporated under reduced pressure to give crude product which was purified by silica gel column chromatography [ethyl acetate:hexane (2:8)] to obtain 4 (7.5 g, 95%) as pale yellow solid. ¹H NMR (CDCl₃, 500 MHz): δ 8.41 (bs, 1H), 7.67 (d, 1H), 7.16-7.13 (m, 1H), 6.87 (d, 1H), 4.38-4.36 (m, 1H), 4.07-4.04 (m, 2H), 3.91 (s, 3H), 3.80 (s, 3H), 2.13 (bs, 1H).

### Synthesis of I-7

To a solution of 4 (7.5 g, 1.0 eq) in methanol (80 mL) was added PTSA (0.1 eq) and palladium on carbon (10%) under nitrogen atmosphere. The reaction mixture was stirred under hydrogen balloon pressure for 16 h at room temperature. After consumption of the starting material (determined by TLC), the reaction mixture was filtered through celite pad and filtrate was evaporated under reduced pressure to give crude product which was purified by silica gel column chromatography [ethyl acetate:hexane (3:7)] to obtain I-7 (0.9 g, 16%) as pale yellow solid. ¹H NMR (CD₃OD, 400 MHz): δ 6.70 (d, 1H), 6.47-6.39 (m, 2H), 3.83-3.72 (m, 3H), 3.69 (s, 3H): Mass: m/z 207.01 (−ve); HPLC purity: 95.81%.

### Synthesis of 3-(2-Hydroxypropan-2-yl)-3, 4-dihydroquinoxalin-2(1H)-one (I-8)

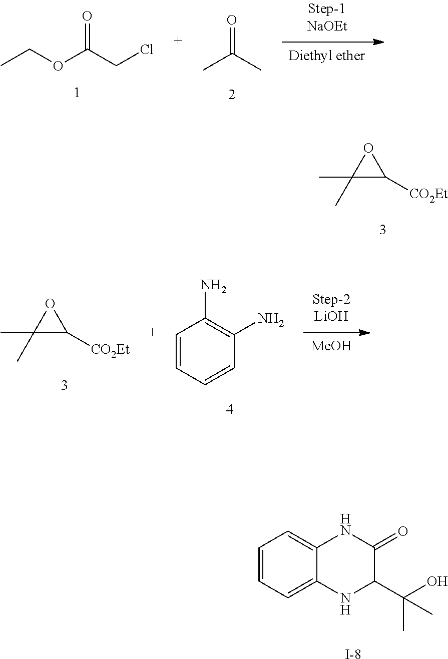

### Synthesis of Ethyl-3, 3-dimethyloxirane-2-carboxylate 3

To a solution of ethyl chloro acetate 1 (10.0 g, 1.0 eq) in diethyl ether (50.0 mL) was added acetone 2 (1.1 eq) under nitrogen atmosphere and the reaction mixture was stirred for 10 min at −10° C. under nitrogen atmosphere. After 5 min, NaOEt (0.8 eq) was added in two lots, keeping the reaction temperature below 5° C. The reaction mixture was stirred for 2 h at −10° C. and then the reaction mixture was slowly warmed to room temperature. The reaction mixture was stirred for 3 h at room temperature. After consumption of the starting material (determined by TLC), the reaction mixture was quenched with NH₄Cl solution and extracted with diethyl ether. The organic layer was separated, dried over Na₂SO₄ and evaporated under reduced pressure to give crude product which was purified by silica gel column chromatography [ethyl acetate:hexane (2:8)] to obtain 3 (8.0 g, 68%) colorless liquid. ¹H NMR (CDCl₃, 400 MHz): δ 4.25 (t, 2H), 4.06 (s, 1H), 1.42 (s, 3H), 1.38 (s, 3H), 1.29 (t, 3H).

### Synthesis of (I-8)

To a solution of 3 (26.0 g, 1.0 eq) in ethanol (100 mL) was added LiGH (0.3 eq) followed by o-phenylene diamine (0.7 eq) at room temperature. The reaction mixture was heated to 80° C. and stirred for 72 h. After consumption of the starting material (determined by TLC), ethanol was evaporated under reduced pressure and diluted with water. The aqueous layer was extracted with ethyl acetate, the organic layer was separated, dried over Na₂SO₄ and evaporated under reduced pressure. The obtained crude material was triturated in ethyl acetate:hexane (1:3) and filtered. The collected solid was dried to obtain I-8 (1.6 g, 4.3%) as pale yellow solid. ¹H NMR (CD₃OD, 400 MHz): δ 7.05-7.01 (m, 1H), 6.97-6.92 (m, 3H), 3.86 (s, 1H), 1.30 (s, 3H), 1.24 (s, 3H): Mass: m/z 207.01 (−ve); HPLC purity: 99.52%.

### Synthesis of (R)-3-(hydroxymethyl)-3, 4-dihydroquinoxalin-2(1H)-one (I-10)

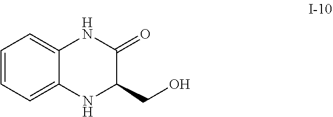 [SMILES: O=C1Nc2ccccc2N[C@@H]1CO]

### Synthesis of I-10

Using procedures described for the synthesis of 1-7, compound I-10 was prepared starting from 1-fluoro-2-nitrobenzene in place of 1-fluoro-4-methoxy-2-nitrobenzene. I-10 was purified by silica gel column chromatography [ethyl acetate:hexane (3:7)] and obtained as a pale yellow solid (1.6 g, 57%). ¹H NMR (CDCl₃, 400 MHz): δ 8.26 (bs, 1H), 6.93-6.89 (m, 1H), 6.78-6.70 (m, 3H), 4.13-4.04 (m, 3H), 3.92-3.87 (m, 1H), 2.67 (bs, 1H): Mass: m/z 176.99 (−ve); HPLC purity: 98.55%.

### Synthesis of (R)-3-(Hydroxymethyl)-7-(trifluoromethyl)-3,4-dihydroquinoxalin-2 (1H)-one (I-11)

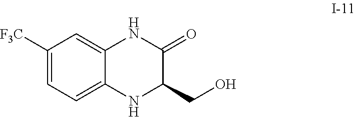 [SMILES: O=C1Nc2cc(C(F)(F)F)ccc2N[C@@H]1CO]

### Synthesis of I-11

Using procedures described for the synthesis of 1-7, compound I-11 was prepared starting from 1-fluoro-2-nitro-4-(trifluoromethyl)benzene in place of 1-fluoro-4-methoxy-2-nitrobenzene. I-11 was purified by silica gel column chromatography [ethyl acetate:hexane (3:7)] and obtained as a pale yellow solid (1.5 g, 47%). ¹H NMR (CD₃OD, 500 MHz): δ 7.07 (m, 1H), 6.96 (d, 1H), 6.77 (d, 1H), 4.04-4.02 (m, 1H), 3.83-3.75 (m, 2H): Mass: m/z 245.08 (−ve); HPLC purity: 96.79%.

### Synthesis of (S)-3-(Hydroxymethyl)-3, 4-dihydroquinoxalin-2(1H)-one (I-12)

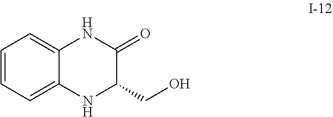 [SMILES: O=C1Nc2ccccc2N[C@H]1CO]

### Synthesis of I-12

Using procedures described for the synthesis of I-7, compound I-12 was prepared starting from L-serine in place of D-serine and 1-fluoro-2-nitrobenzene in place of 1-fluoro-4-methoxy-2-nitrobenzene. 1-12 was purified by silica gel column chromatography [ethyl acetate: hexane (3:7)] and obtained as a pale yellow solid (5.6 g, 50%). ¹H NMR (CDCl₃, 400 MHz): δ 7.95 (bs, 1H), 6.93-6.89 (m, 1H), 6.78-6.70 (m, 3H), 4.13-4.04 (m, 3H), 3.92-3.87 (m, 1H), 2.60-2.57 (m, 1H): Mass: m/z 177.02 (−ve); HPLC purity: 98.10%.

### Synthesis of (S)-3-(Hydroxymethyl)-7-(trifluoromethyl)-3, 4-dihydroquinoxalin-2 (1H)-one (I-13)

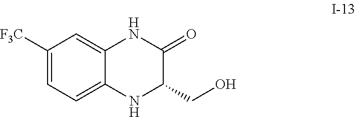 [SMILES: O=C1Nc2cc(C(F)(F)F)ccc2N[C@H]1CO]

### Synthesis of I-13

Using procedures described for the synthesis of I-7, compound I-13 was prepared starting from L-serine in place of D-serine and 1-fluoro-2-nitro-4-(trifluoromethyl)benzene in place of 1-fluoro-4-methoxy-2-nitrobenzene. I-13 was purified by silica gel column chromatography [ethyl acetate:hexane (3:7)] and obtained as a pale yellow solid (5.5 g, 52%). ¹H NMR (CD₃OD, 500 MHz): δ 7.07 (m, 1H), 6.96 (d, 1H), 6.77 (d, 1H), 4.04-4.02 (m, 1H), 3.83-3.75 (m, 2H): Mass: m/z 245.06 (−ve); HPLC purity: 95.48%.

### Example 4: Synthesis of Other Core Compounds

### Synthesis of 2-(3-Aminobenzofuran-2-yl)-1,1,1-trifluoropropan-2-ol (I-20)

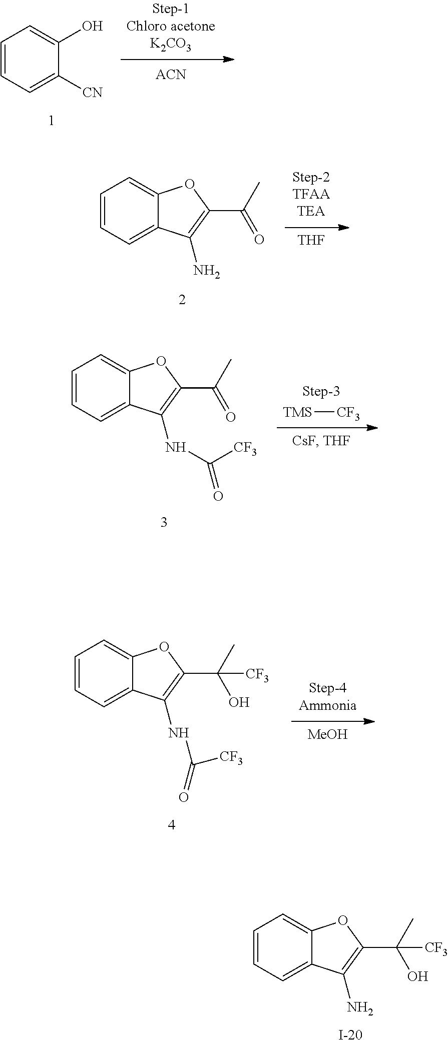

### Synthesis of 1-(3-Aminobenzofuran-2-yl) ethanone 2

To a stirred solution of 2-hydroxybenzonitrile 1 (1.0 g, 1.0 eq) in acetonitrile (16 mL) was added potassium carbonate (1.5 eq) followed by chloro acetone (1.0 eq) at room temperature in a sealed tube. The reaction mixture was stirred for 8 h at 70° C. After consumption of starting material (by TLC), the reaction mixture was cooled to room temperature and diluted with water. The formed precipitate was filtered and dried under vacuum at 40° C. to give brown solid (0.8 g, 54%). ¹H NMR (DMSO-d₆, 400 MHz): δ 7.97 (d, 1H), 7.54-7.48 (m, 2H), 7.27-7.24 (m, 1H), 6.92 (s, 2H), 2.37 (s, 3H); Mass: 176 [+ve]

### Synthesis of N-(2-Acetylbenzofuran-3-yl)-2,2,2-trifluoroacetamide 3

To a stirred solution of 2 (0.3 g, 1.0 eq) in THE (3 mL) was added TEA (2 eq) followed by DMAP (0.1 eq) at room temperature. After stirred the reaction mixture for 10 min, then cooled to 0° C. and trifluoroacetic anhydride (1.6 eq) was added at 0° C. The reaction mixture was stirred for 30 min at room temperature. After consumption of starting material (by TLC), the reaction mass was diluted with water, the formed precipitate was filtered and dried under vacuum to obtain 3 (0.28 g, 61%) as a pale brown fluffy solid. Mass: 270 [−ve].

### Synthesis of 2,2,2-Trifluoro-N-(2-(1,1,1-trifluoro-2-hydroxypropan-2-yl) benzofuran-3-yl) acetamide 4

To a stirred solution of 3 (1.0 g, 1.0 eq) in THE (3.0 mL) was added TMSCF₃ (3.0 eq) and CsF (2.0 eq) at 20° C. The reaction mixture was stirred at room temperature for 6 h. After consumption of starting material (by TLC), the reaction mixture was cooled to 0° C., quenched with cold water and extracted with ethyl acetate. The organic layer was separated, dried over Na₂SO₄ and evaporated under reduced pressure to give 4 (0.34 g, 27%) as a pale brown solid. ¹H NMR (DMSO-d₆, 400 MHz): δ 7.66 (d, 1H), 7.46 (m, 1H), 7.42 (m, 1H), 7.33 (m, 1H), 7.19 (s, H), 1.77 (s, 3H); Mass: 340 [+ve].

### Synthesis of I-20

To a stirred solution of 4 (0.3 g, 1.0 eq) in MeOH (5.0 mL) was added methanolic ammonia (3 mL, 7% solution) at room temperature in a sealed tube. The reaction mixture was stirred at 60° C. for 6 h. After consumption of starting material (by TLC), solvents were evaporated under reduced pressure to obtain I-20 (0.12 mg, 56%) as a brown solid. ¹H NMR (DMSO-d₆, 400 MHz): δ 7.68 (dd, 1H), 7.39 (d, 1H), 7.24 (m, 1H), 7.17 (t, 1H), 6.99 (s, 1H), 4.67 (s, 2H), 1.72 (s, 3H); LCMS: 246 [+ve].

### Synthesis of 2-(5-Aminobenzofuran-6-yl) propan-2-ol (I-22)

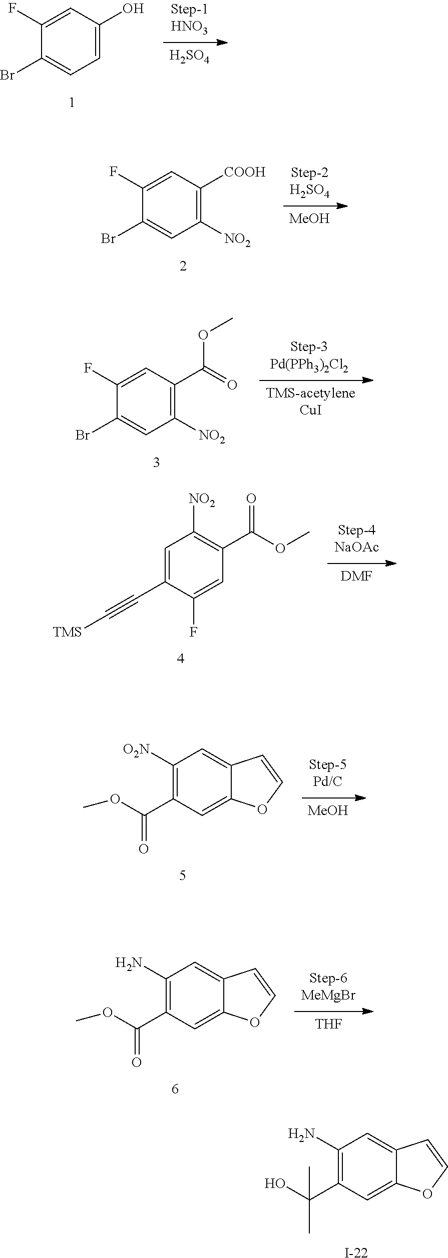

### Synthesis of 4-Bromo-5-fluoro-2-nitro benzoic acid 2

To a stirred solution of 4-bromo-3-fluro benzoic acid (5.0 g, 1.0 eq) in H₂SO₄ (33 mL) was added nitric acid (3 eq) at room temperature and stirred for 2 h. After consumption of starting material (by TLC), the reaction mass was poured into crushed ice, and stirred for 30 min at room temperature, obtained solid was filtered and dried to obtain 2 (5.4 g, 90%) as a white solid. ¹H NMR (DMSO-d₆, 400 MHz): δ 8.51 (d, 1H), 7.90 (d, 1H).

### Synthesis of Methyl 4-Bromo-5-fluoro-2-nitro benzoate 3

To a stirred solution of 2 (5.4 g, 1.0 eq) in methanol (54 mL) was added H₂SO₄ (1 mL, 0.5 eq) at 0° C. The reaction mixture was heated to 80° C. for 16 h. After consumption of starting material (by TLC), the reaction mass was cooled room temperature, methanol was evaporated under reduced pressure. The residue was dissolved in ethyl acetate and washed with saturated sodium bicarbonate solution followed by brine solution. The organic layer was separated, dried over Na₂SO₄ and evaporated under reduced pressure to obtain 3 (4.5 g, 90%) as a colourless liquid. H NMR (CDCl₃, 400 MHz): δ 8.20 (d, 1H), 7.47 (d, 1H), 3.94 (s, 3H).

### Synthesis of Methyl 5-fluoro-2-nitro-4-((trimethylsilyl) ethynyl) benzoate 4

A mixture of 3 (300 mg, 1.0 eq), copper iodide (0.05 eq) and TEA (3 eq) in THE (5 mL) was degassed with nitrogen for 20 min, then Pd (PPh₃)₂Cl₂ (0.05 eq) followed by trimethylsilyl acetylene (2.2 eq) were added at room temperature. The reaction mass was stirred for 6 h. After consumption of starting material (by TLC), the reaction mass was diluted with ethyl acetate. The organic layer was washed with ice cold water (3×20 mL) and followed by brine solution. The ethyl acetate layer was separated, dried over Na₂SO₄ and evaporated under reduced pressure to obtain crude compound which was purified by column chromatography eluted with (ethyl acetate:Hexane, 2:98), pure fraction was distilled to give 4 (190 mg, 59%) as thick syrup. ¹H NMR (CDCl₃, 400 MHz): δ 8.03 (d, 1H), 7.40 (d, 1H), 3.93 (s, 3H), 0.28 (s, 9H).

### Synthesis of Methyl 5-nitrobenzofuran-6-carboxylate 5

To a mixture of 4 (190 mg, 1.0 eq) and sodium acetate (4.0 eq) in DMF (2 mL) was heated to 100° C. and stirred for 16 h. After consumption of starting material (by TLC), water was added to reaction mixture at RT and extracted with MTBE. The organic layer was separated, dried over Na₂SO₄ and evaporated under reduced pressure to obtain crude compound which was purified by column chromatography eluted with (ethyl acetate:Hexane, 10:90), pure fraction was distilled to give 5 (100 mg, 70%) as off white solid. ¹H NMR (CDCl₃, 400 MHz): δ 8.20 (s, 1H), 7.88-7.86 (m, 2H), 6.95-6.94 (m, 1H), 3.92 (s, 3H).

### Synthesis of Methyl 5-aminobenzofuran-6-carboxylate 6

To a solution of 5 (140 mg, 1.0 eq) in methanol (5 mL) was added Pd/C (20 mg, 10 mol %) at room temperature under nitrogen atmosphere. The reaction was stirred under hydrogen atmosphere for 12 h at room temperature. After consumption of starting material (by TLC), the reaction mixture was filtered through celite pad and washed with ethyl acetate twice (20 mL). The filtrate was evaporated under reduced pressure to give crude which was purified by silica gel column chromatography eluted with (ethyl acetate:Hexane, 5:95) pure fraction was distilled to give 6 (50 mg, 58%) as off white solid. ¹H NMR (CDCl₃, 400 MHz): δ 8.01 (s, 1H), 7.63 (d, 1H), 6.81 (s, 1H), 6.58-6.57 (m, 1H), 5.53 (bs, 2H), 3.90 (s, 3H).

### Synthesis of I-22

To solution of 6 (150 mg, 1 eq) in THE (10 mL) was added methyl magnesium bromide (2M solution, 5 eq) for 10 min under nitrogen atmosphere at 0° C. The reaction mass was warmed to room temperature and stir for 4 h. After consumption of starting material (by TLC), the reaction mixture was quenched with NH₄Cl solution and extracted with ethyl acetate. The organic layer was separated, dried over Na₂SO₄ and evaporated under reduced pressure to give crude compound which was purified by silica gel column (ethyl acetate: hexane, 1:5) to obtain I-22 (30 mg, 18%) as a thick syrup. ¹H NMR (CDCl₃, 400 MHz): δ 7.74 (d, 1H), 7.23 (s, 1H), 6.78 (s, 1H), 6.67-6.66 (m, 1H), 5.25 (bs, 2H), 1.55 (s, 6H).

### Synthesis of 2-(5-Amino-2,3-dihydrobenzofuran-6-yl)propan-2-ol (I-23)

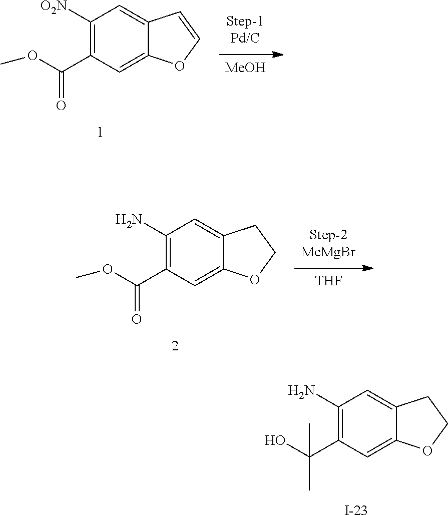

### Synthesis of Methyl 5-amino-2,3-dihydrobenzofuran-6-carboxylate 2

To a solution of 1 (0.7 g, 1.0 eq) in methanol (10 mL) was added Pd/C (70 mg, 10 mol %) at room temperature under nitrogen atmosphere. The reaction was stirred under hydrogen atmosphere for 24 h at room temperature. After consumption of starting material (by TLC), the reaction mixture was filtered through celite pad and washed with ethyl acetate twice (50 mL). The filtrate was evaporated under reduced pressure to give crude which was purified by silica gel column chromatography eluted with (ethyl acetate:Hexane, 5:95) pure fraction was distilled to give 2 (200 mg, 30%) as off white solid. ¹H NMR (CDCl₃, 400 MHz): δ 7.22 (s, 1H), 6.57 (s, 1H), 5.44 (bs, 2H), 4.48 (t, 2H), 3.84 (s, 3H), 3.16-3.11 (m, 2H).

### Synthesis of I-23

To solution of 2 (100 mg, 1 eq) in THE (10 mL) was added methyl magnesium bromide (2M solution, 7 eq) for 10 min under nitrogen atmosphere at 0° C. The reaction mass was warmed to room temperature and stir for 4 h. After consumption of starting material (by TLC), the reaction mixture was quenched with NH₄Cl solution and extracted with ethyl acetate. The organic layer was separated, dried over Na₂SO₄ and evaporated under reduced pressure to give crude compound which was purified by silica gel column (ethyl acetate: hexane, 1:5) to obtain I-23 (20 mg, 20%) as a thick syrup. ¹H NMR (CDCl₃, 400 MHz): δ 6.64 (s, 1H), 6.59 (s, 1H), 4.48 (t, 2H), 3.13-3.09 (m, 2H), 1.64 (s, 6H).

### Synthesis of 2-(3-Amino-5-(trifluoromethyl) pyridin-2-yl) propan-2-ol (I-24)

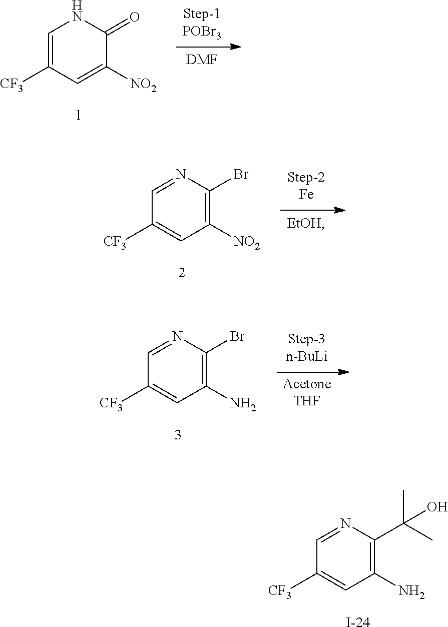

### Synthesis of 2-Bromo-3-nitro-5-(trifluoromethyl) pyridine 2

To a stirred solution of 1 (1.0 g, 1.0 eq) in DMF (5 mL) was added phosphorous oxybromide (1.2 eq) portion wise at 0° C. under nitrogen. The reaction mixture was heated to 80° C. and stirred for 2 h at 80° C. After consumption of starting material (by TLC), the reaction mass was diluted with water, the precipitated solid filtered, washed with water and dried to obtain 2 (0.6 g, 46%) as a light brown solid. LCMS: 273 [+ve].

### Synthesis of 2-Bromo-5-(trifluoromethyl) pyridin-3-amine 3

To a stirred solution of 2 (0.6 g, 1.0 eq) in ethanol (12 mL), water (3 mL) was added iron powder (10.0 eq), con. HCl (0.3 mL) and stirred the reaction mass for 2 h at 80° C. After consumption of starting material (by TLC), the reaction mixture was cooled to room temperature and filtered through celite. Then the filtrate was evaporated under reduced pressure to obtained residue. Which was dissolved with water and extracted with ethyl acetate. The organic layer was separated, dried over Na₂SO₄ and evaporated under reduced pressure to obtain 3 (0.45 g, 84%) as a pale yellow solid. LCMS: 242 [+ve].

### Synthesis of I-24

To a stirred solution of 3 (2 g, 1.0 eq) in THF (50 mL) was added n-butyl lithium (5.0 eq, 1.6M solution) at −70° C. under nitrogen. After stirring for 30 mins, acetone (10.0 eq) was added at −50° C. and stirred the reaction mixture for 2 h at room temperature. After consumption of starting material (by TLC), the reaction mixture was quenched with NH₄Cl solution and extracted with ethyl acetate. The organic layer was separated, dried over Na₂SO₄ and evaporated under reduced pressure to give crude compound which was purified by silica gel column (ethyl acetate: hexane 1:5) to obtain I-24 (160 mg, 8.8%) as a white solid. ¹H NMR (DMSO-d₆, 400 MHz): δ 7.98 (s, 1H), 7.24 (d, 1H), 6.00 (bs, 2H), 5.62 (s, 1H), 1.49 (s, 6H).

### Synthesis of 2-(3-Amino-5-(trifluoromethyl) pyridin-2-yl)-1,1,1-trifluoropropan-2-ol (I-25)

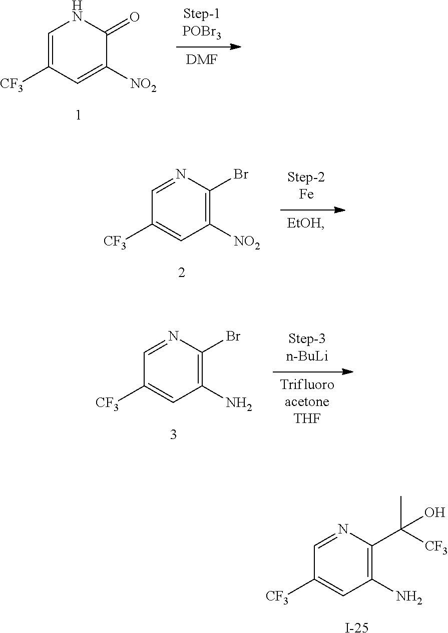

### Synthesis of 2-Bromo-3-nitro-5-(trifluoromethyl) pyridine 2

To a stirred solution of 1 (1.0 g, 1.0 eq) in DMF (5 mL) was added phosphorous oxybromide (1.2 eq) portion wise at 0° C. under nitrogen. The reaction mixture was heated to 80° C. and stirred for 2 h at 80° C. After consumption of starting material (by TLC), the reaction mass was diluted with water, precipitated solid filtered, washed with water and dried to obtain 2 (0.6 g, 46%) as a light brown solid. LCMS: 273 [+ve].

### Synthesis of 2-Bromo-5-(trifluoromethyl) pyridin-3-amine 3

To a stirred solution of 2 (0.6 g, 1.0 eq) in ethanol (12 mL), water (3 mL) was added iron powder (10.0 eq), con. HCl (0.3 mL) and stirred the reaction mass for 2 h at 80° C. After consumption of starting material (by TLC), the reaction mixture was cooled to room temperature and filtered through celite. Then the filtrate was evaporated under reduced pressure, water was added to the residue and extracted with ethyl acetate. The organic layer was separated, dried over Na₂SO₄ and evaporated under reduced pressure to obtain 3 (0.45 g, 84%) as a pale yellow solid. LCMS: 242 [+ve].

### Synthesis of I-25

To a stirred solution of 3 (1 g, 1.0 eq) in THE (50 mL) was added n-butyl lithium (5.0 eq, 1.6M solution) at −70° C. under nitrogen. After stirring for 30 mins at −70° C., trifluoro acetone (10.0 eq) was added at −70° C. and stirred the reaction mixture for 2 h at 0° C. After consumption of starting material (by TLC), the reaction mixture was quenched with NH₄Cl solution and extracted with ethyl acetate. The organic layer was separated, dried over Na₂SO₄ and evaporated under reduced pressure to give crude compound which was purified by silica gel column (1:5 ethyl acetate: hexane) to obtain I-25 (12 mg, 1.1%) as a pale yellow solid. ¹H NMR (CD₃OD, 400 MHz): δ 8.01 (d, 1H), 7.27 (d, 1H), 1.81 (s, 3H).

### Synthesis of 2-(7-Amino-2,3-dihydrobenzo[b] [1,4] dioxin-6-yl) propan-2-ol (I-26)

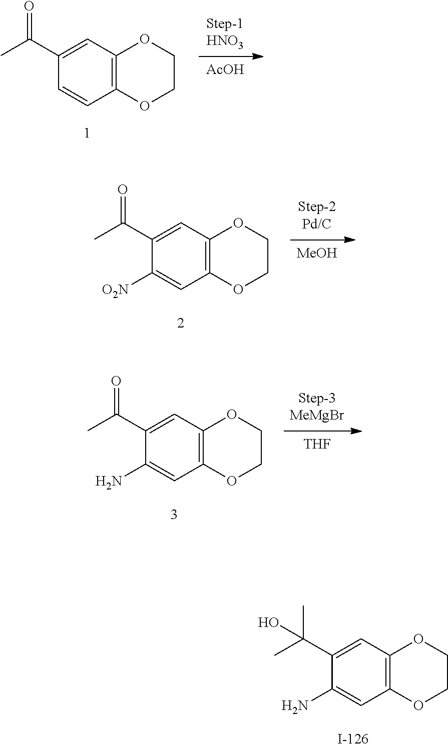

### Synthesis of 1-(7-Nitro-2,3-dihydrobenzo[b] [1,4] dioxin-6-yl) ethenone 2

To a solution of 1-(2,3-dihydrobenzo[b] [1,4] dioxin-6-yl) ethenone 1 (5 g, 1.0 eq) in acetic acid (35 mL) was added fuming nitric acid (15 mL) at 10-15° C. The reaction mixture was stirred for 6 h at room temperature. After consumption of starting material (by TLC), the reaction mixture was allowed to cool to 0° C., poured into ice cold water and extracted with ethyl acetate. The organic layer was separated, dried over Na₂SO₄ and evaporated under reduced pressure to obtain 2 (2.8 g, 45%) as a yellow solid. LCMS: 224 [+ve].

### Synthesis of 1-(7-Amino-2,3-dihydrobenzo[b] 1,4] dioxin-6-yl) ethenone (3)

To a solution of 2 (1.8 g, 1.0 eq) in ethyl acetate (10 mL) and methanol (10 mL) was added Pd/C (200 mg, 10 mol %) at room temperature under nitrogen atmosphere. The reaction was stirred under hydrogen atmosphere for 12 h at room temperature. After consumption of starting material (by TLC), the reaction mixture was filtered through celite pad and washed with ethyl acetate twice (50 mL). The filtrate was evaporated under reduced pressure to give crude which was purified by silica gel column chromatography to obtain 3 (1 g, 64%) as a pale yellow solid. LCMS: 194 [+ve].

### Synthesis of I-26

To solution of 3 (1 g, 1 eq) in THE (20 mL) was added methyl magnesium bromide (2M solution 5 eq) for 10 min under nitrogen atmosphere at 0° C. The reaction mass was warmed to room temperature and stir for 3 h. After consumption of starting material (by TLC), the reaction mixture was quenched with NH₄Cl solution and extracted with ethyl acetate. The organic layer was separated, dried over Na₂SO₄ and evaporated under reduced pressure to give crude compound which was purified by silica gel column (ethyl acetate: hexane, 1:5) to obtain I-26 (120 mg, 15%) as an off white solid. ¹H NMR (CDCl₃, 400 MHz): δ 6.68 (s, 1H), 6.20 (s, 1H), 4.21-4.16 (m, 4H), 1.62 (s, 6H).

### Synthesis of 2-(2-Amino-4,5-difluorophenyl) propan-2-ol (I-27)

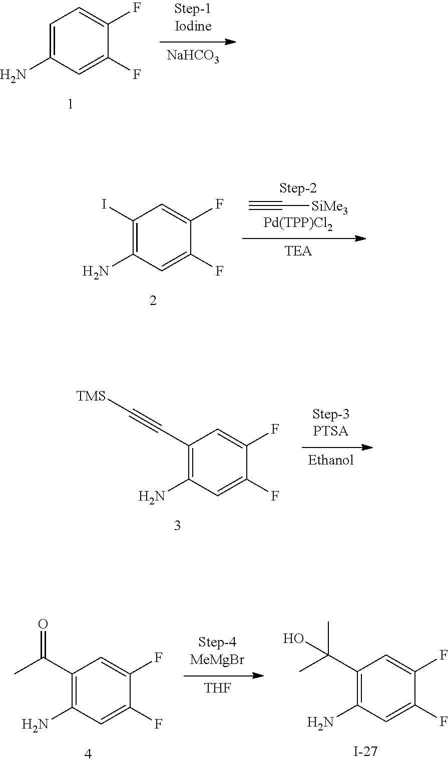

### Synthesis of 4,5-Difluoro-2-iodoaniline 2

A mixture of 3,4-difluoroaniline 1 (2.5 g, 1.0 eq), sodium bicarbonate (3.15 g, 1.5 eq) and iodine (8.25 g, 1.3 eq) in water (125 mL) was stirred for 30 min at room temperature. After consumption of starting material (by TLC) and the reaction mass was diluted with ethyl acetate (100 mL). The organic layer was separated and washed with saturated sodium thiosulphate solution (3×50 mL). The ethyl acetate layer was separated, dried over Na₂SO₄ and evaporated under reduced pressure to obtain 2 (6 g, 95%) as a brown liquid. LC-MS: 256 [+ve].

### Synthesis of 4,5-Difluoro-2-((trimethylsilyl)ethynyl) aniline 3

A mixture of 4,5-difluoro-2-iodoaniline 2 (500 mg, 1.0 eq), copper iodide (68 mg 0.3 eq) in TEA (5 mL) was degassed with nitrogen for 20 min, then Pd(pph₃)₂Cl₂ (0.05 eq) followed by trimethylsilyl acetylene (3 eq) were added at room temperature. The reaction mixture was slowly heated to 40° C. and stirred for 16 h. After consumption of starting material (by TLC), the reaction mass was cooled to room temperature and diluted with ethyl acetate. The organic layer was washed with water (3×5 mL). The ethyl acetate layer was separated, dried over Na₂SO₄ and evaporated under reduced pressure to obtain crude compound which was purified by column chromatography eluted with (ethyl acetate:Hexane, 1:99), pure fraction was distilled to give 3 (250 mg, 56%) as thick syrup. ¹H NMR (CDCl₃, 400 MHz): δ 7.18-7.05 (m, 1H), 6.48-6.44 (m, 1H), 4.17 (bs, 2H), 0.25 (s, 9H).

### Synthesis of 4,5-Difluoro-2-((trimethylsilyl)ethynyl) aniline 4

A mixture of 4,5-difluoro-2-((trimethylsilyl)ethynyl) aniline 3 (250 mg, 1.0 eq), PTSA·H₂O (1 eq) in water (1 mL): ethanol (5 mL) was heated to 80° C. and stirred for 8 h. After consumption of starting material (by TLC), the reaction mass was cooled to room temperature and ethanol was evaporated under reduced pressure. The crude compound was diluted with ethyl acetate and washed with water. The organic layer was separated, dried over Na₂SO₄ and evaporated under reduced pressure to obtain crude compound, which was purified by column chromatography eluted with (ethyl acetate: petroleum ether, 5:95), pure fraction was distilled to furnish 4 (130 mg, 68%) as an off white solid. ¹H NMR (CDCl₃, 400 MHz): δ 7.52-7.47 (m, 1H), 6.43-6.38 (m, 1H), 2.52 (s, 3H).

### Synthesis of I-27

To a stirred solution of 4 (130 mg, 1.0 eq) in THE (5 mL) was added methyl magnesium bromide (5.0 eq, 2M solution) at −10° C. under nitrogen. The reaction mixture was stirred for 1 h at −10° C. After consumption of starting material (by TLC), the reaction mixture was quenched with NH₄Cl solution and extracted with ethyl acetate. The organic layer was separated, dried over Na₂SO₄ and evaporated under reduced pressure to give crude compound which was purified by silica gel column (Ethyl acetate: hexane, 10:90) to obtain I-27 (60 mg, 42%) as thick syrup. ¹H NMR (CD₃OD, 400 MHz): δ 7.35 (bs, 1H), 6.94-6.89 (m, 1H), 6.43-6.38 (m, 1H), 4.60 (bs, 2H), 1.63 (s, 6H).

### Synthesis of 2-(2-Amino-4,5-dichlorophenyl) propan-2-ol (I-28)

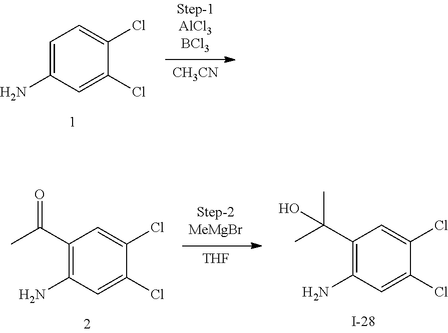

### Synthesis of 1-(2-Amino-4,5-dichlorophenyl) ethenone 2

To a stirred solution of BCl₃ in heptane solution (2.23 g, 1.03 eq) was added 3,4 dichloro aniline (3 g, 1 eq) in acetonitrile (30 mL) drop wise at 0° C. for 10 min under nitrogen atmosphere. AlCl₃ was added to reaction mixture portion wise under nitrogen. The reaction mixture was heated to 80° C. and stirred for 8 h. After consumption of starting material (by TLC), the reaction mass was cooled to 0° C., 4N HCl (30 mL) was added, heated to 100° C. and stirred for 2 h. Volatiles were removed under reduced pressure and extracted with DCM. The organic layer was separated, washed with 2N HCl, dried over Na₂SO₄ and evaporated under reduced pressure to obtain crude compound. This crude compound was purified by column chromatography silica gel eluted with (ethyl acetate: petroleum ether, 1:99), pure fraction was distilled to give 2 (700 mg, 19%) as an off white solid. ¹H NMR (CDCl₃, 400 MHz): δ 7.75 (s, 1H), 6.77 (s, 1H), 6.29 (bs, 2H), 2.54 (s, 3H).

### Synthesis of I-28

To a stirred solution of 2 (150 mg, 1.0 eq) in THE (5 mL) was added methyl magnesium bromide (5.0 eq, 2M solution) at −10° C. under nitrogen. The reaction mass was stirred for 1 h at −10° C. After consumption of starting material (by TLC), the reaction mixture was quenched with NH₄Cl solution and extracted with ethyl acetate. The organic layer was separated, dried over Na₂SO₄ and evaporated under reduced pressure to give crude compound which was purified by silica gel column (ethyl acetate: hexane, 1:9) to obtain I-28(90 mg, 55%) as colorless liquid. ¹H NMR (CD₃OD, 400 MHz): δ 7.14 (s, 1H), 6.78 (s, 1H), 1.56 (s, 6H).

### Synthesis of 2-(6-Aminobenzo[d][1,3] dioxol-5-yl propan-2-ol (I-29)

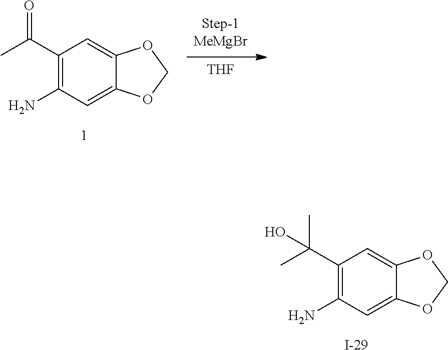

### Synthesis of I-29

To a stirred solution of 1-(6-aminobenzo[d][1,3]dioxol-5-yl) ethanone 1 (0.5 g, 1.0 eq) in THE (15 mL) was added methyl magnesium bromide (4.0 eq, 2M solution) at 0° C. under nitrogen. The reaction was stirred for 2 h at 0° C. After consumption of starting material (by TLC), the reaction mixture was quenched with NH₄Cl solution and extracted with ethyl acetate. The organic layer was separated, dried over Na₂SO₄ and evaporated under reduced pressure to give crude compound which was purified by silica gel column (ethyl acetate: hexane, 1:9) to obtain I-29 (300 mg, 55%) as gummy syrup. ¹H NMR (CDCl₃, 400 MHz): δ 6.66 (s, 1H), 6.23 (s, 1H), 5.83 (s, 2H), 1.62 (s, 6H).

### Synthesis of 2-(2-Amino-4-(trifluoromethyl) phenyl) propan-2-ol (I-30)

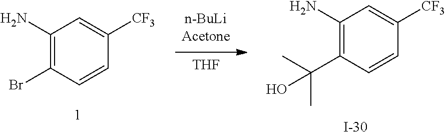

### Synthesis of I-30

To a stirred solution of 1 (1 g, 1.0 eq) in THF (10 mL) was added n-butyl lithium (5.0 eq, 1.6M solution) at −70° C. under nitrogen atmosphere. After stirring for 30 min, acetone (3.0 eq) was added at −70° C. and stirred the reaction mixture for 1 h at room temperature. After consumption of starting material (by TLC), the reaction mixture was quenched with NH₄Cl solution and extracted with ethyl acetate. The organic layer was separated, dried over Na₂SO₄ and evaporated under reduced pressure to give crude compound which was purified by silica gel column (ethyl acetate: hexane, 1:5) to obtain I-30 (200 mg, 22%) as a colourless thick syrup. ¹H NMR (CD₃OD, 400 MHz): δ 7.24 (d, 1H), 6.92 (d, 1H), 6.83-6.80 (m, 1H), 1.61 (s, 6H).

### Synthesis of 2-(2-Amino-4-(trifluoromethyl) phenyl) propan-2-ol (I-31)

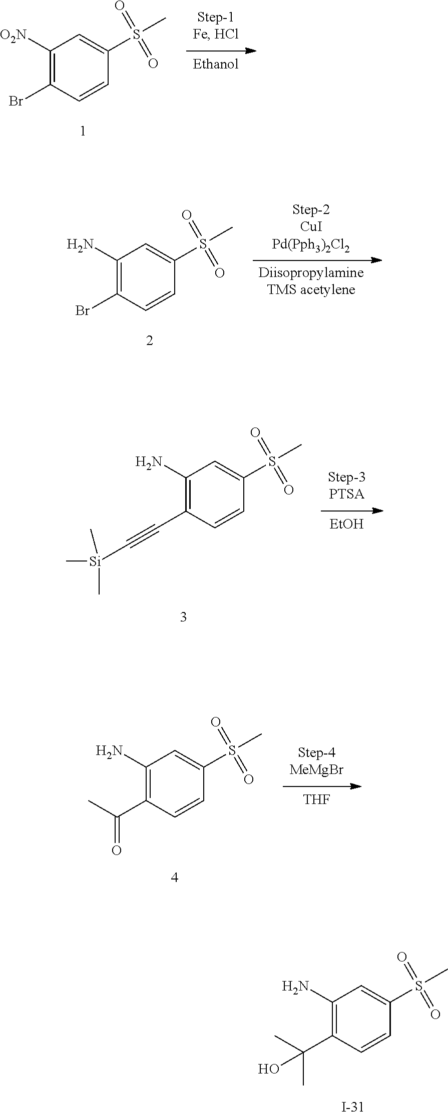

### Synthesis of 2-Bromo-5-(methylsulfonyl) aniline 2

To a stirred solution of 2 (1.0 g, 1.0 eq) in ethanol (3.6 mL) were added iron powder (3.0 eq), con. HCl (0.54 mL) and stirred the reaction mixture for 16 h at 70° C. After consumption of starting material (by TLC), the reaction mixture cooled to room temperature, ethanol was evaporated under reduced pressure. The obtained residue was diluted with water and extracted with ethyl acetate. The organic layer was separated, dried over Na₂SO₄ and evaporated under reduced pressure to obtain 3 (0.82 g, 92%) as a pale brown solid. LCMS: 252 [+ve].

Synthesis of 5-(Methylsulfonyl)-2-((trimethylsilyl)ethynyl) aniline 3: To a stirred solution of 2 (0.1 g, 1.0 eq) in diisopropyl amine (2.0 mL) were added copper iodide (0.02 eq), bis (triphenylphosphine) palladium (II) dichloride (0.04 eq), degassed the contents and TMS acetylene (3.0 eq) was added in a sealed tube. The reaction mixture was stirred for 12 h at 60° C. After consumption of starting material (by TLC), the reaction mixture was cooled to room temperature, water was added and extracted with ethyl acetate. The organic layer was separated, dried over Na₂SO₄ and evaporated under reduced pressure to obtain 3 (0.06 g, 56%) as a pale yellow solid. LCMS: 268 [+ve].

### Synthesis of 1-(2-Amino-4-(methyl sulfonyl) phenyl) ethanone 4

To a stirred solution of 3 (0.8 g, 1.0 eq) in ethanol (3.2 mL), water (0.8 mL) was added PTSA (1.0 eq). The reaction mixture was stirred for 48 h at 70° C. After consumption of starting material (by TLC), water was added and extracted with ethyl acetate. The organic layer was separated, dried over Na₂SO₄ and evaporated under reduced pressure to give 4 (0.483 g, 64%) as a pale yellow solid. LCMS: 214 [+ve].

### Synthesis of I-31

To a stirred solution of 4 (0.3 g, 1.0 eq) in THF (3.0 mL) was added methyl magnesium bromide (4.0 eq, 2M solution) at −70° C. under nitrogen. After addition, the reaction mixture was allowed to reach room temperature and stirred for 2 h. After consumption of starting material (by TLC), the reaction mixture was quenched with NH₄Cl solution and extracted with ethyl acetate. The organic layer was separated, dried over Na₂SO₄ and evaporated under reduced pressure to give crude compound which was purified by silica gel column chromatography (ethyl acetate: hexane, 3:7) to obtain I-31(90 mg, 28%) as a pale yellow solid. ¹H NMR (CD₃OD, 400 MHz): δ 7.32 (d, 1H), 7.18 (d, 1H), 7.08 (dd, 1H), 3.03 (s, 3H), 1.60 (s, 6H).

### Synthesis of 2-(3-Amino-5-methylpyridin-2-yl) propan-2-ol (I-32)

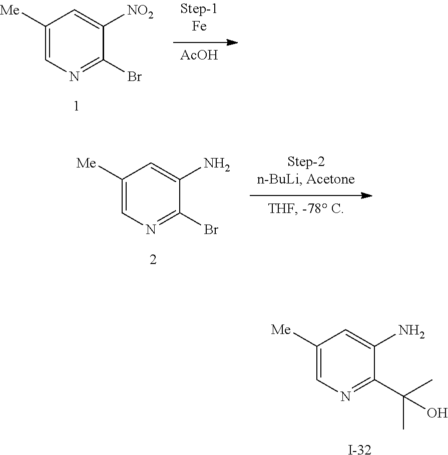

### Synthesis of 2-Bromo-5-methylpyridin-3-amine 2

To a solution of iron powder (4.3 eq) in acetic acid (25.0 mL) was added 1 (5 g, 1.0 eq) at 80° C. under nitrogen. The reaction mixture was stirred for 1 h at 80° C. After consumption of starting material (by TLC), the reaction mixture was cooled to room temperature, diluted with ethyl acetate and filtered through celite. Filtrate was evaporated under reduced pressure, obtained residue was neutralised with NaHCO₃ solution and extracted with ethyl acetate. The organic layer was separated, dried over Na₂SO₄ and evaporated under reduced pressure to obtain 2 (3.7 g, 86%) as a brown solid. LCMS: 187 [+ve].

### Synthesis of 2-(3-Amino-5-methylpyridin-2-yl) propan-2-ol I-32

To a stirred solution of 2 (1 g, 1.0 eq) in THF (20.0 mL) was added n-butyl lithium (5.0 eq, 1.6M solution) at −70° C. under nitrogen. After stirring for 30 min, acetone (10.0 eq) was added at 0° C. and stirred the reaction mixture for 2 h at room temperature. After consumption of starting material (by TLC), the reaction mixture was quenched with NH₄Cl solution at 0° C. and extracted with ethyl acetate. The organic layer was separated, dried over Na₂SO₄ and evaporated under reduced pressure to give crude compound which was purified by silica gel column chromatography (ethyl acetate: hexane, 1:5) to obtain I-32 (20 mg, 1.69%) as a pale yellow solid. ¹H NMR (CD₃OD, 400 MHz): δ 7.59 (d, 1H), 6.89 (d, 1H), 2.20 (s, 3H), 1.58 (s, 6H); LCMS: 167 [+ve]

### Synthesis of 2-(2-Amino-4-(trifluoromethyl) phenyl)-1,1,1-trifluoropropan-2-ol) (I-33)

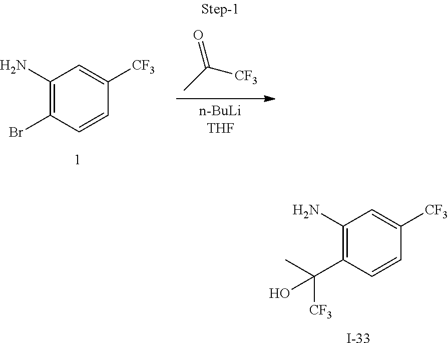

### Synthesis of I-33

To a stirred solution of 1 (1.0 g, 1.0 eq) in THE was added n-butyl lithium (5.0 eq, 1.6M solution) at −70° C. under nitrogen atmosphere. After stirring for 30 min, trifluoro acetone (5.0 eq) was added at −70° C. and slowly raised the reaction temperature to room temperature. The reaction mixture was stirred for 2 h at room temperature. After consumption of starting material (by TLC), the reaction mixture was quenched with NH₄Cl solution and extracted with ethyl acetate. The organic layer was separated, dried over Na₂SO₄ and evaporated under reduced pressure to give crude which was purified by silica gel column chromatography to obtain I-33(15.0 mg, 1.5%) as an off white solid. ¹H NMR (CD₃OD, 400 MHz): δ 7.79 (d, 1H), 7.66-7.65 (m, 1H), 7.54 (s, 1H), 1.92 (s, 3H).

### Synthesis of methyl 3-(4-amino-3-(2-hydroxypropan-2-yl) phenyl)-2-((tert-butoxycarbonyl) amino) propionate·HCl. (I-34 A)

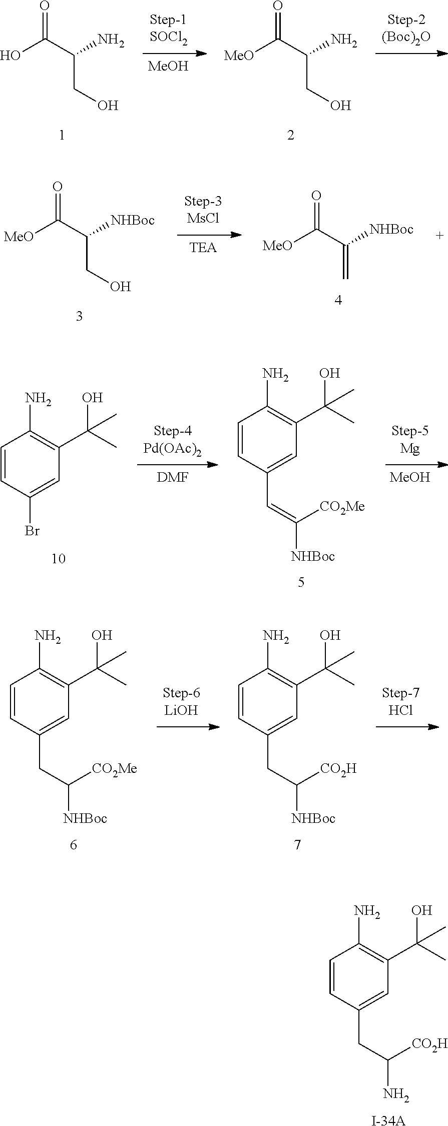

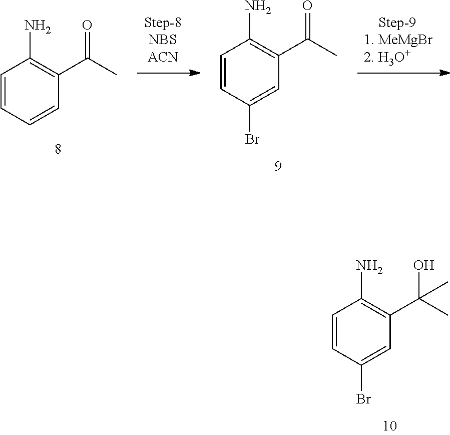

### Synthesis of 1-(2-amino-5-bromophenyl)ethanone 9

To a stirred solution of 8 (20.0 g, 1.0 eq) in acetonitrile (160 mL) was added NBS solution (27.56 g in 150 mL acetonitrile) slowly drop wise at 0° C. over a period of 1 h under nitrogen. The reaction mixture was slowly warmed to room temperature and stirred for 3 h. After consumption of starting material (by TLC), volatiles were evaporated under reduced pressure to obtain crude compound. The crude compound was dissolved in ethyl acetate and washed with water. The organic layer was separated and evaporated to obtain 9 (29.0 g, 920%) as white solid. ¹H NMR (CDCl₃, 400 MHz): δ 7.80 (s, 1H), 7.33 (d, 1H), 6.55 (d, 1H), 6.29 (bs, 2H), 2.55 (s, 3H); Mass: m/z 214.17 (+ve).

### Synthesis of 2-(2-amino-5-bromophenyl) propan-2-ol 10

To a stirred solution of 9 (10.0 g, 1.0 eq) in THF (100 mL) was added 2M methyl magnesium bromide (5.0 eq) solution slowly drop wise over a period of 1 h at 0° C. under nitrogen atmosphere. The reaction mixture was warmed to room temperature and stirred for 2 h. After consumption of starting material (by TLC), the reaction mixture was cooled to 0° C. and quenched with saturated ammonium chloride solution. The organic layer was separated and the aqueous layer was extracted with ethyl acetate twice (2×100 mL). The combined organic layer was dried over sodium sulphate and evaporated under reduced pressure to obtain crude compound. The crude compound was purified by column chromatography to yield 10 (9.5 g, 88%) as a brown solid (low melting solid). ¹H NMR (CDCl₃, 500 MHz): δ 7.20 (d, 1H), 7.14-7.12 (m, 1H), 6.51 (d, 1H), 4.69 (bs, 2H), 1.64 (s, 6H).

### Synthesis of (R)-methyl 2-amino-3-hydroxypropanoate 2

To stirred solution of D-serine 1 (20 g, 190 mmol) in methanol (192 mL) was added thionyl chloride (1.2 eq) slowly drop wise over a period of 15 min at 10° C. The reaction mass was warmed to room temperature and stirred for 2 h. Then reaction was further heated to 80° C. and stirred for 8 h at 80° C. After consumption of starting material (by TLC), the reaction mass was cooled to room temperature and volatiles were evaporated under reduced pressure to obtain 29 g of 2·HCl as an off white solid. The crude compound was used in the next reaction directly without further purification.

### Synthesis of (R)-methyl 2-((tert-butoxycarbonyl)amino)-3-hydroxypropanoate 3

A stirred suspension of 2 (29 g, 243 mmol, 1.0 eq) in DCM (290 mL) was added triethylamine (5.0 eq) slowly drop wise at room temperature over a period of 20 min. Then Boc anhydride (1.1 eq) was slowly added to reaction mass at room temperature for 30 min. The reaction mixture was stirred at room temperature for 3 h. After consumption of starting material (by TLC), then 1N sodium bisulphite solution was added to reaction mixture. The organic layer was separated, washed with 5% NaHCO₃ and 10% citric acid solution. The organic layer was separated, dried over Na₂SO₄ and evaporated under reduced pressure to obtain of 3 (37 g, 70%) as syrup nature. ¹H NMR (CDCl₃, 400 MHz): δ 5.63 (bs, 1H), 4.38 (bs, 1H), 3.97-3.88 (m, 2H), 3.77 (s, 3H), 2.07 (s, 1H), 1.45 (s, 9H).

### Synthesis of methyl 2-((tert-butoxycarbonyl) amino) acrylate 4

To a stirred solution of 3 (5.0 g, 24.1 mmol) in DCM (50 mL) was added mesyl chloride (1.3 eq) drop wise over a period of 5-10 min at −50° C. under nitrogen. The reaction mixture was stirred for 40 min at −50° C., then TEA (3.0 eq) was added drop wise for 10 min at the same temperature. The reaction mass was warmed to room temperature and stirred for 2 h. After consumption of starting material (by TLC), then reaction mixture was diluted with ice cold water. The organic layer was separated and evaporated to obtain crude compound. The crude compound was purified by column chromatography to provide 4 (4.0 g, 87%) as colourless liquid. ¹H NMR (CDCl₃, 400 MHz): δ 7.01 (bs, 1H), 6.16 (bs, 1H), 5.73 (bs, 1H), 3.83 (s, 3H), 1.49 (s, 9H); Mass: m/z 200.1 (−ve).

### Synthesis of (E)-methyl 3-(4-amino-3-(2-hydroxypropan-2-yl) phenyl)-2-((tert-butoxycarbonyl) amino) acrylate 5

To a stirred solution of 10 (2.0 g, 8.60 mmol, 1.0 eq) and 4 (1.3 eq) in DMF (20 mL) in a sealed tube was degassed with nitrogen for 30 min, then palladium acetate (0.1 eq), tri-O-tolyl phosphine (0.2 eq) and triethylamine (1.5 eq) were added to the reaction mixture. The reaction mixture was heated to 100° C. and stirred for 8 h. After consumption of starting material (by TLC), the reaction mass was cooled to room temperature and filtered through celite pad. The filtrate was diluted with ice cold water and extracted with diethyl ether. The organic layer was separated and evaporated under vacuum to obtain crude compound. The crude compound was purified by column chromatography using neutral alumina to furnish 5 (0.8 g, 26%) as pale yellow solid. ¹H NMR (DMSO-d, 400 MHz): δ 8.20 (bs, 1H), 7.52 (s, 1H), 7.29-7.02 (m, 2H), 6.59 (d, 1H), 6.00 (s, 2H), 5.31 (s, 1H), 3.67 (s, 3H), 1.49-1.28 (m, 15H); Mass: m/z 349.29 (−ve).

### Synthesis of methyl 3-(4-amino-3-(2-hydroxypropan-2-yl) phenyl)-2-((tert-butoxycarbonyl) amino) propionate 6

A suspension of 5 (1.3 g, 1.0 eq) and magnesium (10.0 eq) in methanol (26.0 mL) was heated to 80° C. and stirred for 4 h. After consumption of starting material (by TLC), MeOH was evaporated, crude dissolved in ethyl acetate. The organic layer was washed with water, separated and evaporated to obtain crude compound. The crude compound was purified by column chromatography to furnish 6 (1.0 g, 77%) as an off white solid. ¹H NMR (DMSO-d₆, 500 MHz): δ 7.15 (d, 1H), 6.87-6.76 (m, 2H), 6.51 (d, 1H), 5.33 (bs, 2H), 5.17 (bs, 1H), 4.05-4.00 (m, 1H), 3.59 (s, 3H), 2.28-2.66 (m, 2H), 1.46 (s, 6H), 1.34 (s, 9H).

### Synthesis of 3-(4-amino-3-(2-hydroxypropan-2-yl) phenyl)-2-((tert-butoxycarbonyl) amino) propionic acid 7

To a stirred solution of 6 (1.0 g, 1.0 eq) in THF (16 mL) was added lithium hydroxide solution (0.357 g in 4.0 mL water) slowly at 0° C. for 5 min. The reaction mixture was warmed to room temperature and stirred for 4 h. After consumption of starting material (by TLC), reaction mixture was diluted with water and acidified with 20% potassium hydrogen sulphate solution to pH˜5. The aq. layer was extracted with ethyl acetate and evaporated to obtain 7 (0.85 g, 88%) as an off white solid. ¹H NMR (DMSO-d₆, 400 MHz): δ 6.87 (s, 1H), 6.79-6.77 (m, 2H), 6.49 (d, 1H), 5.20 (bs, 1H), 3.96-3.90 (m, 1H), 2.84-2.64 (m, 2H), 1.47 (s, 6H), 1.29 (s, 9H); Mass: m/z 349.29 (−ve).

### Synthesis of I-34A

To a stirred solution of 7 (0.85 g, 2.51 mmol) in 1, 4-dioxane (8.5 mL) was added 4N HCl in 1, 4-dioxane (10.0 eq) slowly drop wise at 0° C. The reaction mass was warmed to room temperature and stirred for 4 h. After consumption of starting material (by TLC), volatiles were evaporated to give crude which was triturated with ethyl acetate to obtain I-34A (0.54 g, 79%) as grey solid. ¹H NMR (D₂O, 400 MHz): δ 7.41 (s, 1H), 7.35-7.34 (m, 2H), 4.26 (t, 1H), 3.32-3.26 (m, 2H), 1.64 (s, 6H); Mass: m/z 237.15 (+ve).

### Synthesis of 2-(3-Aminobenzofuran-2-yl)-1,1,1-trifluoropropan-2-ol (I-35)

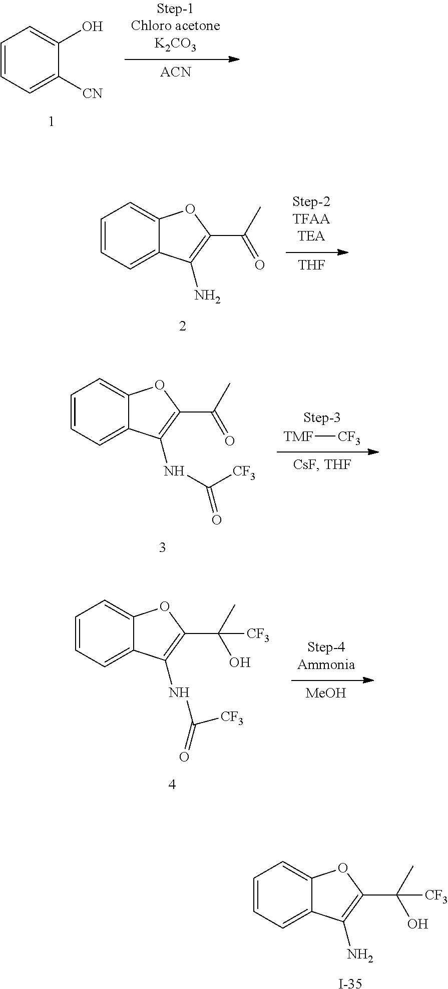

### Synthesis of 1-(3-Aminobenzofuran-2-yl) ethanone 2

To a stirred solution of 2-hydroxybenzonitrile 1 (1.0 g, 1.0 eq) in acetonitrile (16 mL) was added potassium carbonate (1.5 eq) followed by chloro acetone (1.0 eq) at room temperature in a sealed tube. The reaction mixture was stirred for 8 h at 70° C. After consumption of starting material (by TLC), the reaction mixture was cooled to room temperature and diluted with water. The formed precipitate was filtered and dried under vacuum at 40° C. to give brown solid (0.8 g, 54%). ¹H NMR (DMSO-d₆, 400 MHz): δ 7.97 (d, 1H), 7.54-7.48 (m, 2H), 7.27-7.24 (m, 1H), 6.92 (s, 2H), 2.37 (s, 3H); Mass: 176 [+ve].

### Synthesis of N-(2-Acetylbenzofuran-3-yl)-2,2,2-trifluoroacetamide 3

To a stirred solution of 2 (0.3 g, 1.0 eq) in THE (3 mL) was added TEA (2 eq) followed by DMAP (0.1 eq) at room temperature. After stirred the reaction mixture for 10 min, then cooled to 0° C. and trifluoroacetic anhydride (1.6 eq) was added at 0° C. The reaction mixture was stirred for 30 min at room temperature. After consumption of starting material (by TLC), the reaction mass was diluted with water, the formed precipitate was filtered and dried under vacuum to obtain 3 (0.28 g, 61%) as a pale brown fluffy solid. Mass: 270 [−ve].

### Synthesis of 2,2,2-Trifluoro-N-(2-(1,1,1-trifluoro-2-hydroxypropan-2-yl) benzofuran-3-yl) acetamide 4

To a stirred solution of 3 (1.0 g, 1.0 eq) in THE (3.0 mL) was added TMSCF₃ (3.0 eq) and CsF (2.0 eq) at 20° C. The reaction mixture was stirred at room temperature for 6 h. After consumption of starting material (by TLC), the reaction mixture was cooled to 0° C., quenched with cold water and extracted with ethyl acetate. The organic layer was separated, dried over Na₂SO₄ and evaporated under reduced pressure to give 4 (0.34 g, 27%) as a pale brown solid. ¹H NMR (DMSO-d₆, 400 MHz): δ 7.66 (d, 1H), 7.46 (m, 1H), 7.42 (m, 1H), 7.33 (m, 1H), 7.19 (s, H), 1.77 (s, 3H); Mass: 340 [+ve].

### Synthesis of I-35

To a stirred solution of 4 (0.3 g, 1.0 eq) in MeOH (5.0 mL) was added methanolic ammonia (3 mL, 7% solution) at room temperature in a sealed tube. The reaction mixture was stirred at 60° C. for 6 h. After consumption of starting material (by TLC), solvents were evaporated under reduced pressure to obtain I-35 (0.12 mg, 56%) as a brown solid. ¹H NMR (DMSO-d₆, 400 MHz): δ 7.68 (dd, 1H), 7.39 (d, 1H), 7.24 (m, 1H), 7.17 (t, 1H), 6.99 (s, 1H), 4.67 (s, 2H), 1.72 (s, 3H); LCMS: 246 [+ve].

### Synthesis of 2-(3-Amino-5-chloropyridin-2-yl) propan-2-ol (I-36)

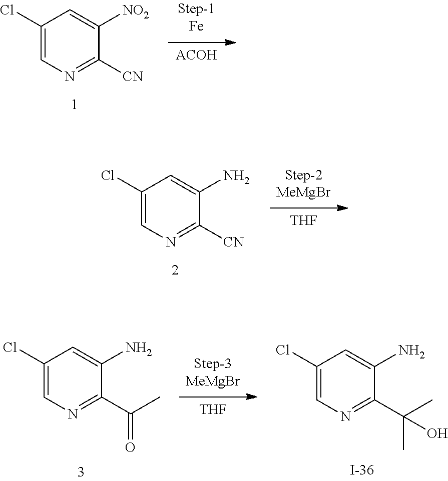

### Synthesis of 3-Amino-5-chloropicolinonitrile 2

To a solution of iron powder (5.5 eq) in acetic acid (20.0 mL) was added 1 (2 g, 1.0 eq) at 80° C. under nitrogen atmosphere. The reaction mixture was stirred for 30 min at 80° C. After consumption of starting material (by TLC), the reaction mixture was diluted with water and extracted with ethyl acetate. The organic layer was separated, dried over Na₂SO₄ and evaporated under reduced pressure to give crude compound which was purified silica gel column chromatography (ethyl acetate: hexane, 3:7) to obtain 2 (0.64 g, 38%) as a pale yellow solid. LCMS: 152 [−ve].

### Synthesis of 1-(3-Amino-5-chloropyridin-2-yl) ethanone 3

To a stirred solution of 2 (0.4 g, 1.0 eq) in THE (4.0 mL) was added methyl magnesium bromide (4.0 eq, 2M solution) at 0° C. under nitrogen. After addition, the reaction mixture was allowed to room temperature and stirred for 2 h. After consumption of starting material (by TLC), the reaction mixture was quenched with NH₄Cl solution and extracted with ethyl acetate. The organic layer was separated, dried over Na₂SO₄ and evaporated under reduced pressure to give crude compound which was purified by silica gel column chromatography (ethyl acetate: hexane, 1:5) to obtain 3 (180 mg, 41%) as a pale brown solid. ¹H NMR (CD₃OD, 400 MHz): δ 7.80 (d, 1H), 7.21 (d, 1H), 2.59 (s, 3H); LCMS: 171 [+ve].

### Synthesis of I-36

To a stirred solution of 3 (0.18 g, 1.0 eq) in THE (3.6 mL) was added methyl magnesium bromide (3.0 eq, 2M solution) at 0° C. under nitrogen atmosphere. After addition, the reaction mixture was allowed to room temperature and stirred for 3 h. After consumption of starting material (by TLC), the reaction mixture was quenched with NH₄Cl solution at 0° C. and extracted with ethyl acetate. The organic layer was separated, dried over Na₂SO₄ and evaporated under reduced pressure to give crude compound which was purified by silica gel column (ethyl acetate: hexane, 1:5) to obtain I-36 (70 mg, 36%) as a pale yellow solid. ¹H NMR (CD₃OD, 400 MHz): δ 7.66 (d, 1H), 7.02 (d, 1H), 1.56 (s, 6H); LCMS: 186 [−ve].

### Synthesis of 2-(3-Amino-5-bromopyridin-2-yl) propan-2-ol (I-37)

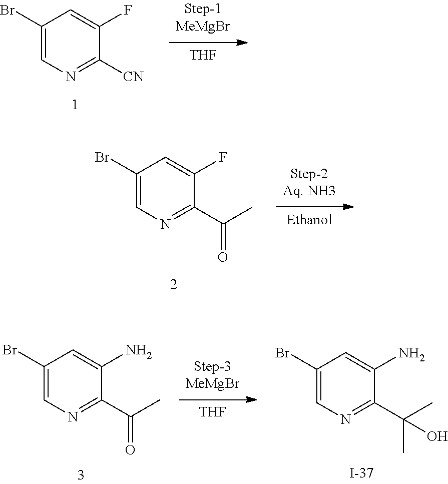

### Synthesis of 1-(5-Bromo-3-fluoropyridin-2-yl) ethanone 2

To a stirred solution of 1 (2.0 g, 1.0 eq) in THE (20.0 mL) was added methyl magnesium bromide (3.0 eq, 2M solution) at 0° C. under nitrogen. After addition, the reaction mixture was allowed to room temperature and stirred for 2 h. After consumption of starting material (by TLC), the reaction mixture was quenched with NH₄Cl solution at 0° C. and extracted with ethyl acetate. The organic layer was separated, dried over Na₂SO₄ and evaporated under reduced pressure to give crude compound which was purified by silica gel column chromatography (ethyl acetate: hexane, 1:5) to obtain 2 (730 mg, 34%) as a pale brown solid. LCMS: 218 [+ve].

### Synthesis of 1-(3-Amino-5-bromopyridin-2-yl) ethanone 3

To a stirred solution of 2 (0.73 g, 1.0 eq) in ethanol (20.0 mL) was added aq. ammonia (20.0 mL) in a sealed tube. The reaction mixture was stirred for 12 h at 80° C. After consumption of starting material (by TLC), the reaction mixture was cooled to room temperature and ethanol was evaporated. Water was added to the residue and extracted with ethyl acetate. The organic layer was separated, dried over Na₂SO₄ and evaporated under reduced pressure to give crude compound which was purified by silica gel column (ethyl acetate: hexane, 1:5) to obtain 3 (0.15 g, 21%) as a yellow solid. LCMS: 215 [+ve].

### Synthesis of I-37

To a stirred solution of 1 (150 mg, 1.0 eq) in THF (3.0 mL) was added methyl magnesium bromide (3.0 eq, 2M solution) at 0° C. under nitrogen. After addition allowed the reaction mixture to reach room temperature and stirred for 2 h. After consumption of starting material (by TLC), the reaction mixture was quenched with NH₄Cl solution at 0° C. and extracted with ethyl acetate. The organic layer was separated, dried over Na₂SO₄ and evaporated under reduced pressure to give crude compound which was purified by silica gel column (ethyl acetate: hexane, 3:7) to obtain I-37(50 mg, 43%) as a pale yellow solid. ¹H NMR (CD₃OD, 400 MHz): δ 7.74 (d, 1H), 7.17 (d, 1H), 1.56 (s, 6H); LCMS: 230 [+ve].

### Example 5: Experimental Conditions Used for Initial Trapping Experiments

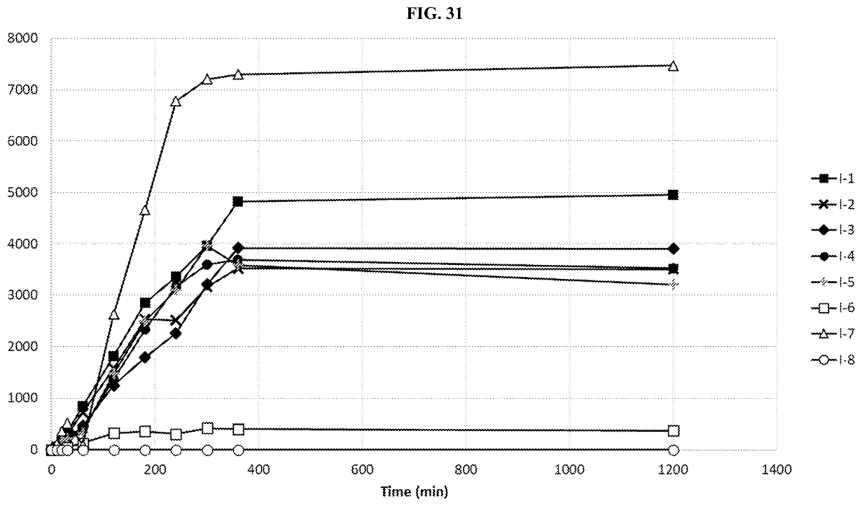

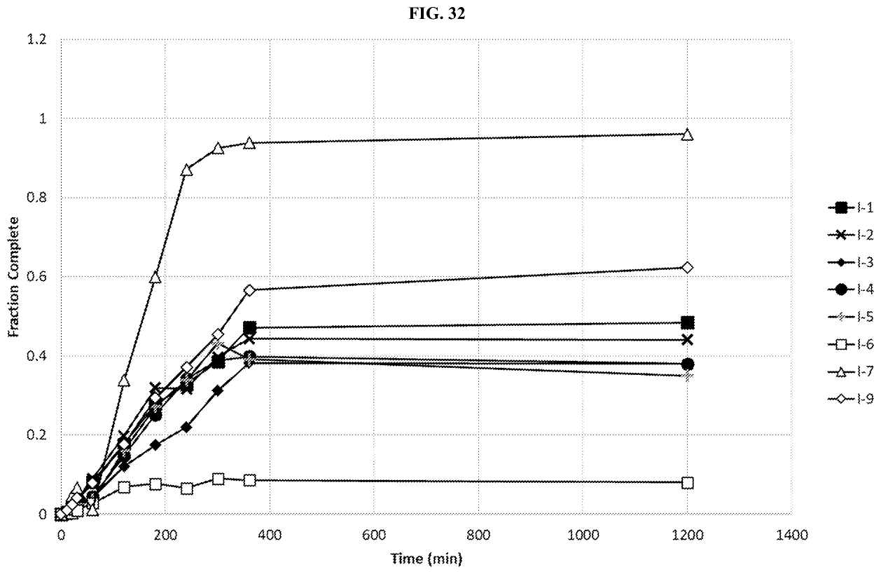

Each of compounds I-1 through I-13 (0.01 moles) was tested for reactivity using 4-hydroxynonenal (4-HNE) (0.006 mol) as a model aldehyde. The experimental compound was dissolved in a 20% solution of Captisol in phosphate buffer (1 mL, pH=7.2). Then, 4-HNE in ethanol (10 mg/mL solution) was added to the solution. The ensuing reaction was monitored by HPLC analysis. After approximately 24 hours, excess formic acid was added to complete the reaction and provide the final composition of product:starting material. The relative ratio of experimental compound/4-HNE adduct to final composition was plotted against time to provide an indication of reaction rate and level of reaction completeness. Results are shown in FIG. 31 and FIG. 32.

### Example 6: Experimental Conditions Used for Additional Trapping Experiments

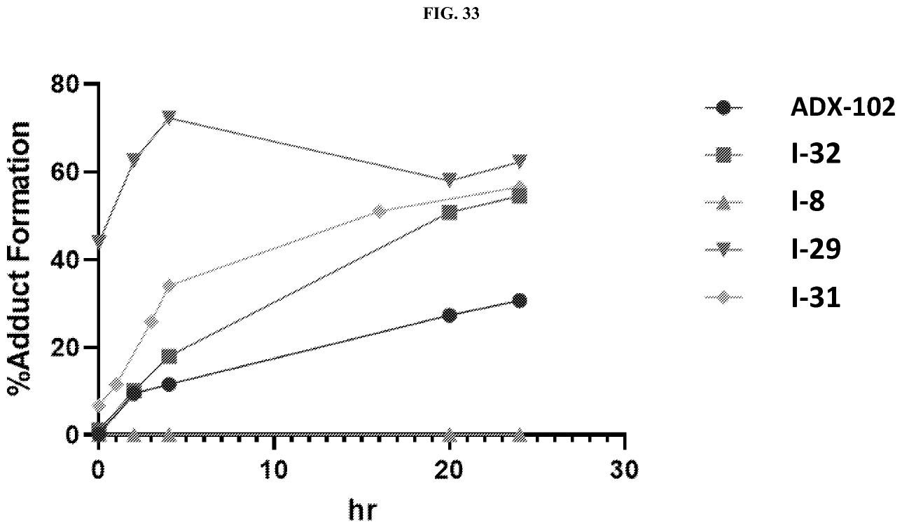

Each of compounds ADX-102, I-32, I-8, I-29, and I-31 (0.01 moles) was tested for reactivity using 4-hydroxynonenal (4-HNE) (1.5 eq) as a model aldehyde. ADX-102 is also known as Reproxalap. Each compound was dissolved in a 20% solution of Captisol in phosphate buffer (1 mL, pH=7.2). Then, 4-HNE in ethanol (10 mg/mL solution) was added to the solution. The ensuing reaction was monitored by LCMS analysis. The relative ratio of experimental compound/4-HNE adduct to final composition was plotted against time to provide an indication of reaction rate and level of reaction completeness. Results are shown in FIG. 33, which shows rates of formation of aldehyde adducts over a 24-h time period. It was found that all samples bind (positive increase in product HPLC peak over time). I-29 demonstrated the best binding, followed by I-31, which demonstrated slightly better binding than I-32.

### Example 7: Evaluation of Prophylactic Anti-Inflammatory Activity of Test Compounds in Acute LPS-Induced Sepsis in C57BL/6 Mice

### Summary

The primary objective of this study was to collect plasma samples of lipopolysaccharide (LPS) challenged C57BL/6 mice after dose of test compounds to provide a cytokine profile as impacted by prophylactic treatments. This study models acute sepsis, which is a systemic inflammatory syndrome initiated by Gram-negative and Gram-positive bacteria and fungi which infect the lungs, abdomen, bloodstream, and renal or genitourinary tracts. Sepsis patients ultimately die of multiorgan failure which is caused by extensive tissue hypo-oxygenation due to ongoing microvascular leakage, disseminated intravascular coagulation, compromised energy production, and metabolic alterations. Sepsis is characterized by an early systemic inflammatory response phase featuring symptoms, such as tachycardia, fever, hyperventilation, and activation of the complement and coagulation cascades. However, it is now appreciated that a compensatory anti-inflammatory response phase follows, characterized by neuroendocrine-mediated immunosuppression. Since these processes are the result of interaction between cells of inflammation and organs, investigation of the treatment of this syndrome requires the use of intact animal models. The use of ten mice per group allows useful statistical modelling of the results.

For the data shown in Table 2A, 103 mice (female, 18-22 gram, C57BL/6) were purchased from ENVIGO. The mice were housed in 20 cages of 5 mice per cage with 1 cage of 3 mice as extra. The cages had filter tops and autoclaved bedding, and the animals were placed in quarantine with daily inspection. The treatment groups are detailed in Table 2A below. 10 mice were included in each group. The mice were dosed by oral gavage (PO) at 10 ml/kg as per the table below. MC=methyl cellulose.

TABLE 2A

| | | Dose | Route of | LPS | |
|---|---|---|---|---|---|
| Group | Cmpd # | (mg/kg) | Admin | Injection | Formulation |
| 1 | Vehicle | 10 ml/kg | PO | 0.5 hr | 0.5% MC |
| 2 | I-3 | 200 | PO | 0.5 hr | 0.5% MC |
| 3 | I-14 | 200 | PO | 0.5 hr | 0.5% MC |
| 4 | I-35 | 200 | PO | 0.5 hr | 0.5% MC |
| 5 | I-16 | 200 | PO | 0.5 hr | 0.5% MC |
| 7 | I-8 | 200 | PO | 0.5 hr | 0.5% MC |
| 8 | I-11 | 200 | PO | 0.5 hr | 0.5% MC |
| 10 | I-34 | 200 | PO | 0.5 hr | 0.5% MC |
| 11 | I-7 | 200 | PO | 0.5 hr | 0.5% MC |
| 12 | I-22 | 100 | PO | 0.5 hr | 0.5% MC |
| 13 | I-23 | 100 | PO | 0.5 hr | 0.5% MC |
| 14 | I-24 | 200 | PO | 0.5 hr | 0.5% MC |
| 15 | I-26 | 125 | PO | 0.5 hr | 0.5% MC |
| 16 | I-27 | 200 | PO | 0.5 hr | 0.5% MC |
| 17 | I-28 | 200 | PO | 0.5 hr | 0.5% MC |
| 18 | I-29 | 200 | PO | 0.5 hr | 0.5% MC |
| 19 | I-31 | 200 | PO | 0.5 hr | 0.5% MC |
| 20 | I-33 | 75 | PO | 0.5 hr | 0.5% MC |
| 22 | I-32 | 75 | PO | 0.5 hr | 0.5% MC |

For the data shown in Table 2B, 185 mice (female, 18-22 gram, C57BL/6) were purchased from ENVIGO. The mice were housed in 37 cages of 5 mice per cage. The cages had filter tops and autoclaved bedding, and the animals were placed in quarantine with daily inspection. The treatment groups are detailed in Table 2B below. 10 mice were included in each group. The mice were dosed by oral gavage (P0) at 10 ml/kg as per the table below. MC=methyl cellulose.

TABLE 2A

| | | Dose | Route of | LPS | |
|---|---|---|---|---|---|
| Group | Cmpd # | (mg/kg) | Admin | Injection | Formulation |
| 1 | Vehicle | 10 ml/kg | PO | 0.5 hr | 0.5% MC |
| 3 | I-3 | 200 | PO | 0.5 hr | 0.5% MC |
| 4 | I-8 | 200 | PO | 0.5 hr | 0.5% MC |
| 5 | I-16 | 200 | PO | 0.5 hr | 0.5% MC |
| 6 | I-22 | 100 | PO | 0.5 hr | 0.5% MC |
| 7 | I-23 | 100 | PO | 0.5 hr | 0.5% MC |
| 8 | I-24 | 200 | PO | 0.5 hr | 0.5% MC |
| 9 | I-26 | 125 | PO | 0.5 hr | 0.5% MC |
| 10 | I-27 | 200 | PO | 0.5 hr | 0.5% MC |
| 11 | I-28 | 200 | PO | 0.5 hr | 0.5% MC |
| 12 | I-29 | 200 | PO | 0.5 hr | 0.5% MC |
| 13 | I-31 | 200 | PO | 0.5 hr | 0.5% MC |
| 14 | I-33 | 75 | PO | 0.5 hr | 0.5% MC |
| 16 | I-32 | 75 | PO | 0.5 hr | 0.5% MC |
| 18 | I-8 | 200 | PO | 0.5 hr | 0.5% MC |

### Procedure

At T=0 h, the mice were dosed with one of the test compounds as described in the above table.

At T=0.5 h, the mice were injected IP with 1.5 mg/kg MPS (Sigma).

At T=6.5 h, all mice were anesthetized and exsanguinated into pre-chilled EDTA-treated tubes. The blood was processed to plasma which was stored in labeled 0.5 polypropylene snap-cap tubes (0.5 mL Eppendorf Safe-Lock Tubes (Fisher Scientific) at −80° C.

Preparation of vehicle: 1.0 gram of methyl cellulose (Sigma) was dissolved in 200 mL of water (USP purified) to make a solution of 0.5% methyl cellulose in water. Preparation of LPS solution: 6 mg of LPS (from Escherichia Coli 055:B5, Cat #L2880, Sigma Lot #057m4013) was dissolved in 20 ml of saline to give a LPS solution of 0.3 mg/ml. All mice were injected with 5 ml/kg (1.5 mg/kg) at the scheduled time by intraperitoneal injection.

### Cytokine Panel Summary

A 32-plex cytokine panel was obtained from Eve Technologies. Each cytokine was assessed based against a 7 point curve range in duplicate. The average of the two replicate analyses was used for statistics. Unpaired t-tests were performed using Excel™ contrasting vehicle against each treatment group. Column plots of the treatment groups for each cytokine are shown in the Figures. One star indicates P<0.05. Two stars indicates P<0.01. Three stars indicates P<0.001. Four stars indicates P<0.0001. Most, but not all, statistically significant results are indicated with star(s).

A heatmap of significant cytokine changes is shown in Table 3A and Table 3B below.

TABLE 3A

| | 100 | 200 | 200 | 100 | 200 | 200 | 100 | 100 |
|---|---|---|---|---|---|---|---|---|
| | mpk | mpk | mpk | mpk | mpk | mpk | mpk | mpk |
| | IP | PO | PO | IP | PO | PO | PO | PO |
| Description | I-1 | I-2 | I-3 | I-9 | I-7 | I-16 | I-22 | I-23 |
| Eotaxin | * | ** | ** | | | | | |
| G-CSF | * | | | | | | | |
| GM-CSF | ** | ** | *** | *** | | ** | * | * |
| IFNy | **** | | **** | | | | | |
| IL-1a | | | *** | | | | | |
| IL-1B | ** | ** | **** | * | * | | | |
| IL-2 | | | | | | | | |
| IL-3 | **** | | **** | | | * | | |
| IL-4 | | | | | | | | |
| IL-5 | **** | ** | *** | ** | | ** | | |
| IL-6 | | **** | ** | | * | | | |
| IL-7 | * | ** | | *** | | | | |
| IL-9 | | | *** | | | | | |
| IL-10 | **** | | * | **** | | ** | | |
| IL-12 (p40) | **** | * | **** | **** | ** | | | |
| IL-12 (p70) | * | | | | | | | |
| IL-13 | | **** | *** | | | | | |
| IL-15 | ** | ** | ** | ** | | | | |
| IL-17 | *** | *** | *** | | | | | |
| KC | | *** | ** | | * | | | |
| LIF | | ** | * | | | | | |
| LIX | | | * | | | | | |
| MCP-1 | | *** | **** | * | * | * | | |
| M-CSF | | | ** | | *** | **** | ** | |
| MIP-1a | ** | | | ** | | | | |
| MIP-1B | | | | | | | | |
| MIP-2 | | *** | | * | | | | |
| RANTES | | | | | | | | |
| TNFa | | **** | ** | | * | | | |
| VEGF | | | * | | | | | |

TABLE 3B

| | 200 | 125 | 200 | 200 | 200 | 200 | 75 | 75 |
|---|---|---|---|---|---|---|---|---|
| | mpk | mpk | mpk | mpk | mpk | mpk | mpk | mpk |
| | PO | PO | PO | PO | PO | PO | PO | PO |
| Description | I-24 | I-26 | I-27 | I-28 | I-29 | I-31 | I-32 | I-33 |
| Eotaxin | | | | | | | | |
| G-CSF | | | | | | | | |
| GM-CSF | | **** | * | ** | **** | *** | | |
| IFNy | | | | | | | **** | |
| IL-1a | | | | | | | | |
| IL-1B | | | | ** | ** | ** | *** | ** |
| IL-2 | | | | | ** | | | |
| IL-3 | | | | | | * | | ** |
| IL-4 | | | | | | | | |
| IL-5 | | | | | ** | ** | *** | ** |
| IL-6 | | | | | ** | | | |
| IL-7 | | | | | | | | |
| IL-9 | | * | | | *** | | | |
| IL-10 | | | | * | | | * | |
| IL-12 (p40) | * | | | | *** | **** | ** | *** |
| IL-12 (p70) | | | | * | **** | *** | * | * |
| IL-13 | | | | * | | | *** | ** |
| IL-15 | | | | | | ** | | |
| IL-17 | | | | | | * | * | |
| KC | | | * | *** | * | * | | ** |
| LIF | | | ) | | **** | **** | **** | **** |
| LIX | | | | | | | | |
| MCP-1 | | | | * | | ** | | |
| M-CSF | * | | | | | * | **** | |
| MIP-1a | | | | | | | | |
| MIP-1B | | | | | | | | |
| MIP-2 | | | | | * | ** | | ** |
| RANTES | | | | | | | | |
| TNFa | | | | | | | | |
| VEGF | | | | | | | | |

### CONCLUSIONS

Significant changes were observed for the treatment groups vs. vehicle for several cytokines.

We claim:

1. A compound of formula VIII: 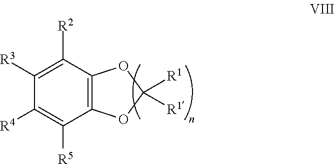
or a pharmaceutically acceptable salt thereof, wherein:
each of R², R³, R⁴, and R⁵ is independently hydrogen, deuterium, halogen, —NH₂, —CN, —OR, —SR, —S(O)R, —S(O)₂R, optionally substituted C₁₋₆ aliphatic, or 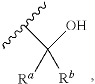
provided that one of R², R³, R⁴, and R⁵ is —NH₂ and another one of R², R³, R⁴, and R⁵ is 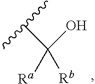
and the —NH₂ and the 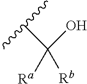
are attached to adjacent carbon atoms;
R¹ and R^{1′} are each independently hydrogen, deuterium, or C₁₋₆ alkyl;
R^{a} is C₁₋₄ aliphatic optionally substituted with 1, 2, or 3 deuterium or halogen atoms; and
R^{b} is C₁₋₄ aliphatic optionally substituted with 1, 2, or 3 deuterium or halogen atoms; or R^{a} and R^{b}, taken together with the carbon atom to which they are attached, form a 3-8 membered cycloalkyl or heterocyclyl ring containing 1-2 heteroatoms each independently selected from nitrogen, oxygen, and sulfur;
each R is independently selected from hydrogen, deuterium, and an optionally substituted group selected from: C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated monocyclic heterocyclic ring having 1-5 heteroatoms independently selected from nitrogen, oxygen, and sulfur; and a 8- to 10-membered bicyclic heteroaryl ring having 1-5 heteroatoms independently selected from nitrogen, oxygen, and sulfur; and
n is 1, 2, or 3.

2. The compound of claim 1, wherein R¹ and R^{1′} are H.

3. The compound of claim 1, wherein R² is hydrogen, deuterium, halogen, —CN, —OMe, —S(C₁₋₆ alkyl), —S(O)(C₁₋₆ alkyl), or C₁₋₆ alkyl.

4. (canceled)

5. The compound of claim 1, wherein R³ is 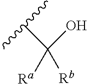
and R⁴ is —NH₂.

6. (canceled)

7. (canceled)

8. A compound of formula IX: 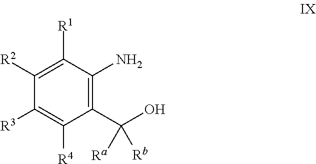
or a pharmaceutically acceptable salt thereof, wherein:
each of R¹, R², R³, and R⁴ is independently hydrogen, deuterium, halogen, —NH₂, —CN, —OR, —SR, —S(O)R, —S(O)₂R, or optionally substituted C₁₋₆ aliphatic;
R^{a} is C₁₋₄ aliphatic optionally substituted with 1, 2, or 3 deuterium or halogen atoms; and
R^{b} is C₁₋₄ aliphatic optionally substituted with 1, 2, or 3 deuterium or halogen atoms; or R^{a} and R^{b}, taken together with the carbon atom to which they are attached, form a 3-8 membered cycloalkyl or heterocyclyl ring containing 1-2 heteroatoms each independently selected from nitrogen, oxygen, and sulfur; and
each R is independently selected from hydrogen, deuterium, and an optionally substituted group selected from: C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated monocyclic heterocyclic ring having 1-5 heteroatoms independently selected from nitrogen, oxygen, and sulfur; and a 8- to 10-membered bicyclic heteroaryl ring having 1-5 heteroatoms independently selected from nitrogen, oxygen, and sulfur.

9. The compound of claim 8, wherein R¹ is hydrogen, deuterium, halogen, —CN, —OMe, —S(C₁₋₆ alkyl), —S(O)(C₁₋₆ alkyl), or C₁₋₆ alkyl.

10. The compound of claim 9, wherein R² is —S(O)R or —S(O)₂R.

11. The compound of claim 10, wherein R¹ and R⁴ are H.

12. A compound of formula X: 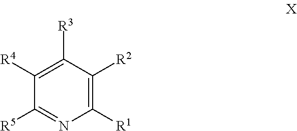
or a pharmaceutically acceptable salt thereof, wherein:
each of R¹, R², R³, R⁴, and R⁵ is independently hydrogen, deuterium, halogen, —NH₂, —CN, —OR, —SR, —S(O)R, —S(O)₂R, optionally substituted C₁₋₆ aliphatic, or 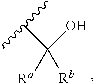
provided that one of R¹, R², R³, R⁴, and R⁵ is —NH₂ and another one of R¹, R², R³, R⁴, and R⁵ is 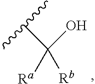
and the —NH₂ and the 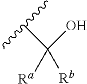
are attached to adjacent carbon atoms;
R^{a} is C₁₋₄ aliphatic optionally substituted with 1, 2, or 3 deuterium or halogen atoms; and
R^{b} is C₁₋₄ aliphatic optionally substituted with 1, 2, or 3 deuterium or halogen atoms; or R^{a} and R^{b}, taken together with the carbon atom to which they are attached, form a 3-8 membered cycloalkyl or heterocyclyl ring containing 1-2 heteroatoms each independently selected from nitrogen, oxygen, and sulfur; and
each R is independently selected from hydrogen, deuterium, and an optionally substituted group selected from: C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated monocyclic heterocyclic ring having 1-5 heteroatoms independently selected from nitrogen, oxygen, and sulfur; and a 8- to 10-membered bicyclic heteroaryl ring having 1-5 heteroatoms independently selected from nitrogen, oxygen, and sulfur.

13-16. (canceled)

17. A compound of formula I: 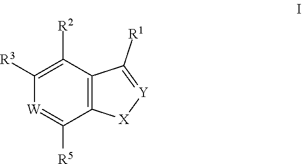
or a pharmaceutically acceptable salt thereof, wherein:
W is N or CR⁴;
X is S, NH, or O;
Y is N or CR⁶;
provided that, if X is S or O, then Y is CR⁶;
each of R¹, R², R³, R⁴, R⁵, and R⁶ is independently hydrogen, deuterium, halogen, —NH₂, —CN, —OR, —SR, optionally substituted C₁₋₆ aliphatic, or 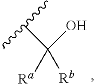
provided that one of R¹, R², R³, R⁴, R⁵, and R⁶ is —NH₂ and another one of R¹, R², R³, R⁴, R⁵, and R⁶ is 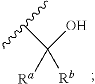
R^{a} is C₁₋₄ aliphatic optionally substituted with 1, 2, or 3 deuterium or halogen atoms;
R^{b} is C₁₋₄ aliphatic optionally substituted with 1, 2, or 3 deuterium or halogen atoms;
or R^{a} and R^{b}, taken together with the carbon atom to which they are attached, form a 3-8 membered cycloalkyl or heterocyclyl ring containing 1-2 heteroatoms each independently selected from nitrogen, oxygen, and sulfur; and
each R is independently selected from hydrogen, deuterium, and an optionally substituted group selected from: C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated monocyclic heterocyclic ring having 1-5 heteroatoms independently selected from nitrogen, oxygen, and sulfur; and a 8- to 10-membered bicyclic heteroaryl ring having 1-5 heteroatoms independently selected from nitrogen, oxygen, and sulfur.

18-33. (canceled)

34. The compound of claim 17, wherein the compound is a compound of formula II-a, II-b, II-c, II-d, II-e, II-f, II-g, II-h, II-i, or II-j: 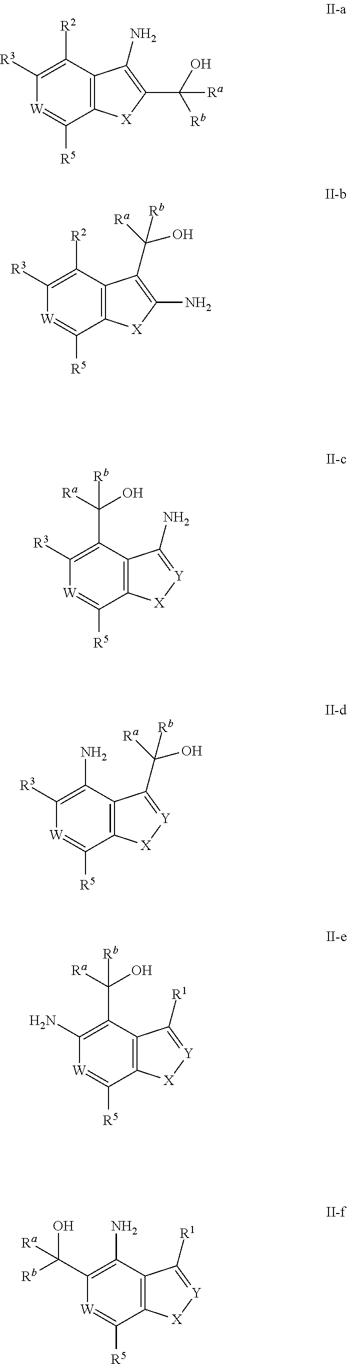 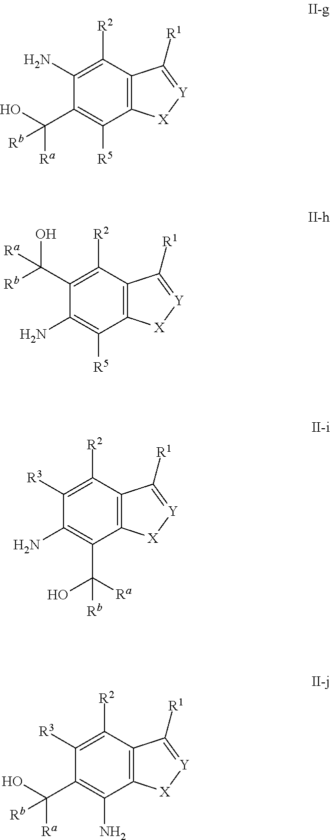
or a pharmaceutically acceptable salt thereof.

35-37. (canceled)

38. A compound of formula VI: 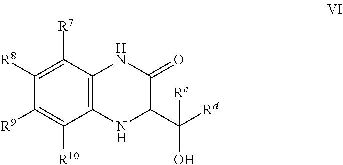
or a pharmaceutically acceptable salt thereof, wherein:
each of R⁷, R⁸, R⁹, and R¹⁰ is independently hydrogen, deuterium, halogen, —N(R)₂, —CN, —OR, —SR, or optionally substituted C₁₋₆ aliphatic;
R^{c} is hydrogen or C₁₋₄ aliphatic optionally substituted with 1, 2, or 3 deuterium or halogen atoms;
R^{d} is hydrogen or C₁₋₄ aliphatic optionally substituted with 1, 2, or 3 deuterium or halogen atoms; or R^{c} and R^{d}, taken together with the carbon atom to which they are attached, form a 3-8 membered, saturated cycloalkyl or heterocyclyl ring containing 1-2 heteroatoms each independently selected from nitrogen, oxygen, and sulfur; and
each R is independently selected from hydrogen, deuterium, and an optionally substituted group selected from: C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated monocyclic heterocyclic ring having 1-5 heteroatoms independently selected from nitrogen, oxygen, and sulfur; and a 8- to 10-membered bicyclic heteroaryl ring having 1-5 heteroatoms independently selected from nitrogen, oxygen, and sulfur.

39-44. (canceled)

45. A compound selected from any one of the compounds depicted in Table 1, or a pharmaceutically acceptable salt thereof.

46. A pharmaceutical composition comprising a compound according to claim 8 and a pharmaceutically acceptable adjuvant, carrier, or vehicle.

47. (canceled)

48. A method of treating macular degeneration or a retinal disease whose etiology involves accumulation of A2E and/or lipofuscin in a subject, comprising administering to the subject an effective amount of a compound according to claim 8, or a pharmaceutically acceptable salt thereof, and thereby reducing the level of A2E accumulation relative to the level of A2E accumulation in said subject without administration of the compound or pharmaceutically acceptable salt thereof.

49. A method of treating, preventing, or reducing a risk of a disease, disorder, condition, or cosmetic indication in which aldehyde toxicity is implicated in a subject in need thereof, comprising administering topically or systemically to the subject a compound according to claim 8 or a pharmaceutically acceptable salt thereof.

50-65. (canceled)

66. The method of claim 49, wherein the disease, disorder, or condition is an autoimmune, immune-mediated, inflammatory, cardiovascular, or neurological disease, or diabetes, metabolic syndrome, or a fibrotic disease.

67. The method of claim 66, wherein the disease, disorder, or condition is disease, disorder or disease is non-alcoholic fatty liver disease (NAFLD), non-alcoholic steatohepatitis, inflammatory bowl disease, Crohn's disease, ulcerative colitis (UC), psoriasis, IBS (irritable bowel syndrome or spastic colon), ankylosing spondylitis, osteoporosis, rheumatoid arthritis (RA), psoriatic arthritis, chronic obstructive pulmonary disease (COPD), atherosclerosis, pulmonary arterial hypertension, pyridoxine-dependent epilepsy, atopic dermatitis, rosacea, multiple sclerosis (MS), systemic lupus erythematosus (SLE), lupus nephritis, sepsis, eosinophilic esophagitis, chronic kidney disease (CKD), fibrotic renal disease, chronic eosinophilic pneumonia, extrinsic allergic alveolitis, pre-eclampsia, endometriosis, polycystic ovary syndrome (PCOS), reduced female fertility, reduced sperm viability and motility, cyclophosphamide-induced hemorrhagic cystitis; or light chain deposition disease, IgA nephropathy, end stage renal disease, gout, pseudogout, diabetic nephrophathy, diabetic neuropathy, traumatic brain injury, noise-induced hearing loss, Alzheimer's Disease, Parkinson's Disease, Huntington Disease, amyotrophic lateral sclerosis, primary biliary cirrhosis, primary sclerosing cholangitis, uterine leiomyoma, sarcoidosis, or chronic kidney disease.

68-71. (canceled)

72. A method of reducing levels of one or more toxic aldehydes in a subject, comprising administering to the subject a compound of claim 8, or a pharmaceutically acceptable salt thereof.

73. The method of claim 72, wherein the toxic aldehyde is selected from formaldehyde, acetaldehyde, acrolein, glyoxal, methylglyoxal, hexadecanal, octadecanal, hexadecenal, succinic semi-aldehyde, malondialdehyde, 4-hydroxynonenal, 4-hydroxy-2E-hexenal, 4-hydroxy-2E,6Z-dodecadienal, retinaldehyde, leukotriene B4 aldehyde, and octadecenal.
